(19) United States
(12) Patent Application Publication
(10) Pub. No.: US 2020/0165302 A1
(43) Pub. Date: May 28, 2020

(54) MODIFIED VSV-G AND VACCINES THEREOF

(71) Applicant: UNIVERSITÉ CATHOLIQUE DE LOUVAIN, Louvain-la-Neuve (BE)

(72) Inventors: Gaëlle VANDERMEULEN, Hoeilaart (BE); Laure LAMBRICHT, Louvain-la-Neuve (BE); Véronique PRÉAT, Kraainem (BE)

(73) Assignee: UNIVERSITÉ CATHOLIQUE DE LOUVAIN, Louvain-la-Neuve (BE)

(21) Appl. No.: 16/332,909

(22) PCT Filed: Sep. 14, 2017
(86) PCT No.: PCT/EP2017/073119

§ 371 Date: Mar. 13, 2019

(30) Foreign Application Priority Data
Sep. 14, 2016 (EP) 16188736.9

(51) Int. Cl. C07K 14/005; A61K 39/205; A61P 35/00; A61K 39/395; C12N 7/00; A61K 39/00

(52) U.S. Cl. CPC C07K 14/005; A61K 39/205; A61P 35/00; A61K 39/3955; C12N 2760/20234; A61K 2039/54; A61K 2039/572; C12N 2760/20222; A61K 2039/53; C12N 7/00

(57) ABSTRACT

A modified vesicular stomatitis virus glycoprotein (VSV-G) that includes at least one peptide, preferably an antigen or fragment thereof, nucleic acid sequence coding therefor, and vectors containing the nucleic acid sequence. Also vaccines and methods for the treatment of a disease or condition, in particular a cancer or an infectious disease.

### FIELD OF INVENTION

The present invention relates to the field of methods and related compositions for the preparation and administration of vaccines, such as nucleic acid-based vaccines, for the treatment of one or more diseases.

### BACKGROUND OF INVENTION

Cancer remains one of the leading causes of death in the modern world. The standard treatments currently practiced in the clinic, including surgery, radiation, and chemotherapy, have shown limited success. These therapies are usually only effective against early stage localized tumors and rarely against later staged, metastatic malignancies, leading to frequent relapses.

Furthermore, various agents used in radiation and chemotherapy are damaging to normal tissues, which may lead to prominent side effects.

For a few decades, vaccines have been applied as therapeutic strategies, harnessing the power of the immune system to activate T cells against infected cells and cancers. For example, DNA vaccines are developed against various diseases including influenza and HIV-1 (Ulmer et al., 1993. Science. 259:1745-1749; Wang et al., 1993. PNAS. 90:4156-4160). These findings, along with the discovery and identification of cancer antigens, have propelled the investigation and development of DNA vaccines against cancer (Wang et al., 1999. Immunol. Rev. 170:85-100).

DNA vaccines are more cost effective compared to other vaccines, such as recombinant protein, tumor cells, or viral vectors. Recent advancements in molecular biology and recombinant technologies along with the increasing identification of tumor antigens provide the tools for plasmid gene manipulation. Genes in DNA vaccines can be designed to encode different antigens as well as various other immunomodulatory molecules to manipulate the resulting immune responses.

Despite all the advantages, DNA vaccines have had limited success in producing therapeutic effects against most cancers due to poor immunogenicity. Various strategies have been investigated to enhance the potency of DNA vaccines. Plasmids encoding antigens have been designed to promote antigen expression and presentation.

Several components derived from bacteria or viruses are able to interact with the immune system, acting as adjuvants. For example, cholera or Clostridium difficile toxins have been shown to enhance the immunogenicity of mucosal antigens (Mohan et al., 2013. Indian. J. Med. Res. 138(5):779-795). Unmethylated CpG motifs that are present on bacterial DNA have a strong stimulatory influence on the immune system and can be used to modulate the immunogenicity of DNA vaccines (Klinman et al., 1997. J. Immunol. 158(8):3635-9). More recently, the efficacy of cancer DNA vaccine was improved by the coadministration of a plasmid encoding

HIV-1 Gag viral capsid protein (Lambricht et al., 2016. Mol. Ther. 24(9):1686-96). Vesicular stomatitis virus glycoprotein (VSV-G) has also been used as an adjuvant to enhances DNA vaccine potency (Marsac et al., 2002. J. Virol. 76(15):7544-7553; Mao et al., 2010. J. Virol. 84(5):2331-2339). In addition, VSV-G has been shown as having fusogenic properties that contribute to control tumor growth and mediate cancer cells killing (Bateman et al., 2000. Cancer Res. 60(6):1492-1497; Bateman et al., 2002. Cancer Res. 62(22):6566-6578).

The poor immunogenicity of DNA vaccines has driven a shift towards mRNA vaccine, another nucleic acid-based technology with interesting properties for immunization (Schlake et al., 2012. RNA Biol. 9(11): 1319-1330; Sahin et al., 2014. Nat Rev Drug Discov. 13(10):759-80; McNamara et al., 2015. J Immunol Res. 2015:794528). RNA vaccines are attractive because they retain the same appealing characteristics as DNA vaccines but also offer some additional benefits. Unlike DNA, RNA only needs to gain entry into the cytoplasm, where translation occurs, in order to transfect a cell. Moreover, RNA cannot integrate into the genome and therefore has no oncogenic potential.

VSV-G is frequently used for pseudotyping because viruses bearing a VSV-G envelope are able to transduce an extensive range of cell types. To alter the tropism of viral vectors, VSV-G mutants have been constructed by inserting tumor targeting ligands (Guibinga et al., 2004. Mol. Ther. 9(1):76-84; Ammayappan et al., 2013. J. Virol. 87(24):13543-13555). Modified VSV-G was also obtained to construct virus-based vaccine carrying a neutralizing epitope from HIV-1 intended to promote generation of neutralizing antibodies (Grigera et al., 1996. J. Virol. 70(12):8492-8501; Schlehuber and Rose, 2004. J. Virol. 78(10):5079-5087). Finally, co-administration of a plasmid coding for an antigen and a plasmid encoding VSV-G has been shown to slow down cancer progression and to prolong survival (Mao et al., 2010. J Virol. 84(5): 2331-2339).

Here, the Applicant surprisingly demonstrates that a VSV-G protein comprising epitopes inserted into specific sites retains its immunogenic properties. Consistently, the Applicant shows that administration of a nucleic acid coding for such VSV-G protein generates a strong immune response against these epitopes. In particular, DNA immunization with a VSV-G sequence comprising tumoral epitopes leads to a significant effect on tumor growth.

Therefore, the present invention relates to a nucleic acid encoding a vesicular stomatitis virus glycoprotein comprising at least one heterologous peptide, such as an antigen or a fragment thereof, and uses thereof for immunization.

### SUMMARY

The present invention relates to an isolated nucleic acid sequence coding for a modified vesicular stomatitis virus glycoprotein (VSV-G), comprising at least one tumor antigen or fragment thereof.

In one embodiment, the at least one tumor antigen or fragment thereof comprises at least one epitope. In one embodiment, the at least one tumor antigen or fragment thereof is a neoantigen.

In one embodiment, the at least one antigen or fragment thereof is inserted into VSV-G at an amino acid position selected from the group consisting of positions 18, 51, 55, 191, 196, 217, 368 and C-terminal, and combinations thereof, wherein position numbering is with respect to vesicular stomatitis Indiana virus (VSIV) glycoprotein amino acid sequence (SEQ ID NO: 1).

The present invention further relates to a vector comprising the nucleic acid sequence of the invention.

The present invention further relates to a dendritic cell population transfected by the nucleic acid of the invention or by the vector of the invention.

The present invention further relates to a modified vesicular stomatitis virus glycoprotein (VSV-G) encoded by the isolated nucleic acid sequence of the invention.

The present invention further relates to a composition comprising the isolated nucleic acid sequence of the invention, the vector of the invention, the dendritic cell of the invention or the modified VSV-G of the invention.

The present invention further relates to a vaccine comprising the isolated nucleic acid sequence of the invention, the vector of the invention, the dendritic cell of the invention or the modified VSV-G of the invention, and optionally at least one adjuvant.

The present invention further relates to the modified VSV-G of the invention, the nucleic acid sequence coding therefor, the vector containing the nucleic acid sequence coding therefor, the dendritic cell population transfected by the nucleic acid sequence coding therefor, or the vaccine comprising said modified VSV-G, nucleic acid sequence, vector or dendritic cell population and optionally at least one adjuvant, for use in preventing and/or treating a disease or condition in a subject in need thereof.

In one embodiment, the vaccine for use according to the present invention is a polynucleotide vaccine. In one embodiment, the vaccine for use according to the present invention is a protein vaccine.

In one embodiment, the disease or condition is a cancer or an infectious disease.

In one embodiment, the modified VSV-G of the invention, the nucleic acid sequence coding therefor, the vector containing the nucleic acid sequence coding therefor, the dendritic cell population transfected by the nucleic acid sequence coding therefor, or the vaccine comprising said modified VSV-G, nucleic acid sequence, vector or dendritic cell population for use according to the present invention is to be administered to the subject by intramuscular injection, intradermal injection, intratumoral injection, peritumoral injection, gene gun, electroporation or sonoporation.

In one embodiment, the modified VSV-G of the invention, the nucleic acid sequence coding therefor, the vector containing the nucleic acid sequence coding therefor, the dendritic cell population transfected by the nucleic acid sequence coding therefor, or the vaccine comprising said modified VSV-G, nucleic acid sequence, vector or dendritic cell population for use according to the present invention is to be administered before, concomitantly or after one or more checkpoint blockade antibodies.

### Definitions

In the present invention, the following terms have the following meanings:
“Peptide” refers to a linear polymer of amino acids of less than 50 amino acids linked together by peptide bonds; a “polypeptide” refers to a linear polymer of at least 50 amino acids linked together by peptide bonds; and a “protein” specifically refers to a functional entity formed of one or more peptides or polypeptides, and optionally of non-polypeptides cofactors.
“Signal peptide”, also called signal sequence, targeting signal, localization signal, localization sequence, transit peptide, leader sequence or leader peptide, refers to a peptide, present at the N-terminus or at the C-terminus of a protein, used to address it to a particular cellular compartment, such as the nucleus, the endoplasmic reticulum, the Golgi, and the like. In one embodiment, the signal peptide of the invention comprises from 4 to 35 amino acids.
“Antigen” refers to any molecule that can initiate a cellular and/or humoral immune response in a subject, leading to the stimulation of B and/or T lymphocytes. In one embodiment, an antigen is capable of being bound by an antibody or T cell receptor. The structural aspect of an antigen, e.g., three-dimensional conformation or modification (such as, e.g., phosphorylation), that gives rise to a biological response, is referred to herein as “epitope”, “antigenic determinant” or “antigen epitopic fragment”.
“Neoantigen” or “neoantigenic” refers to a class of tumor antigens that arises from one or several tumor-specific mutation(s) which alter(s) the amino acid sequence of genome encoded proteins.
The terms “epitope”, “antigenic determinant” and “antigen epitopic fragment” can be used interchangeably. They refer to the part of an antigen that is recognized by the immune system, specifically by antibodies, B cells or T cells. Epitopes can be formed both from contiguous amino acids or noncontiguous amino acids juxtaposed by tertiary folding of a protein (therefore referred to as “conformational epitope”). Epitopes formed from contiguous amino acids are typically retained on exposure to denaturing solvents, whereas conformational epitopes are typically lost on treatment with denaturing solvents. They define the minimum binding site for an antibody, B cell or T cell, and thus represent the target of specificity of an antibody, B cell or T cell.
“T-cell epitope” refers to an epitope that can be bound by MHC molecules of class I or II in the form of a peptide-presenting MHC molecule or MHC complex and then, in this form, be recognized and bound by naïve T cells, cytotoxic CD8 T cells or T helper CD4 cells. T cell epitopes may be presented by MHC class I for CD8 T cell recognition (therefore referred to as CD8 T cell epitopes), by MHC class II for CD4 T cell recognition (therefore referred to as CD4 T cell epitopes or helper T cell epitopes), or by both.
“Pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA.
“Immunogenic composition” is a composition that comprises an antigenic molecule where administration of the composition to a subject results in the development in the subject of a humoral and/or a cellular immune response to the antigenic molecule of interest. In one embodiment, the immunogenic composition may be introduced directly into a recipient subject, such as by injection, inhalation, oral, intranasal and mucosal administration.
“Vaccine” refers to any preparation comprising substance or group of substances meant to cause the immune system of a subject to respond to pathogens, such as bacteria or viruses, or to a tumor. Prophylactic vaccines are used to prevent a subject from ever having a particular disease or to only have a mild case of the disease. Such prophylactic vaccines usually comprise the pathogen responsible for the disease, either live and weakened or killed, or components thereof, purified or recombinant. Therapeutic vaccines are intended to treat specific diseases in a subject, in particular cancer. Such therapeutic anti-cancer vaccines comprise a tumor-antigen or tumor-antigens, eliciting an immune response directed against the tumor cells.
“Adjuvant” refers to a molecule that stimulates the immune response against an antigen and/or that modulates the immune response so as to obtain the expected response. In particular, the addition of adjuvants in vaccine formulations aims to improve, accelerate, shift and/or extend the specific immune response directed against the antigen(s) comprised in the vaccine formulations. The advantages of adjuvants include enhancing the immunogenicity of antigens, changing the nature of the immune response, reducing the amount of antigen(s) required to induce an effective immunization, reducing the frequency of booster immunizations, and enhancing the immune response in the elderly and the immunocompromised.
“Genetic adjuvant” refers to any biologically active factor, such as a cytokine, an interleukin, a chemokine, a ligand, and optimally combinations thereof, which is expressed by a vector, and which, when administered with a DNA vaccine encoding an antigen, enhances the antigen-specific immune response. Desirable genetic adjuvants include, but are not limited to, DNA sequences encoding: GM-CSF, interferons (IFNs) (for example, IFN-α, IFN-β and IFN-γ), interleukins (ILs) (for example, IL-1β, IL-2, IL-10, IL-12, IL-13), TNF-α, and combinations thereof. The genetic adjuvants may also be immunostimulatory polypeptide from Parapox virus, such as a polypeptide of Parapox virus strain D1701 or NZ2 or Parapox immunostimulatory polypeptides B2WL or PP30. Still other such biologically active factors that enhance the antigen-specific immune response may be readily selected by one of skill in the art, and a suitable plasmid vector containing the same factors constructed by known techniques (for a review on genetic adjuvant for DNA vaccines, see Calarota & Weiner, 2004. Expert Rev. Vaccines. 3:S135-49; Calarota & Weiner, 2004. Immunol. Rev. 199:84-99; Kutzler & Weiner, 2004. J. Clin. Invest. 14(9):1241-4).
In one embodiment, the genetic adjuvant is not encoded by the polynucleotide or vector coding for a modified VSV-G according to the invention. In another embodiment, the genetic adjuvant is encoded by the polynucleotide or vector coding for a modified VSV-G according to the invention. According to this embodiment, the genetic adjuvant can be under the control of its own promoter; or the genetic adjuvant can be under the control of the same promoter as the modified VSV-G according to the invention, separated therefrom by an Internal Ribosome Entry Site (IRES).
“Dendritic cells” refers to antigen-presenting cells of the immune system which present cytoplasmic branched projections called dendrites at certain development stages. Dendritic cells have the particular function to trigger the adaptive immune response induced in response to an antigen.
“Subject” refers to an animal, preferably a mammal, more preferably a human.
In one embodiment, a subject may be a mammal. Mammals include, but are not limited to, all primates (human and non-human), cattle (including cows), horses, pigs, sheep, goats, dogs, cats, and any other mammal which is awaiting the receipt of, or is receiving medical care or was/is/will be the object of a medical procedure, or is monitored for the development of a disease.
In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. In one embodiment, the subject is an adult (for example a subject above the age of 18). In another embodiment, the subject is a child (for example a subject below the age of 18). In one embodiment, the subject is a male. In another embodiment, the subject is a female.
“Treating” or “treatment” or “alleviation” refers to both therapeutic treatment and prophylactic or preventative measures; wherein the object is to prevent or slow down (lessen) the targeted pathologic condition or disorder, such as for example a cancer or an infection. Those in need of treatment include those already with the disorder as well as those prone to have the disorder or those in whom the disorder is to be prevented. A subject or mammal is successfully “treated” for a specific disease or condition, such as for example a cancer or an infection if, after receiving a therapeutic amount of modified VSV-G, polynucleotide, composition, or vaccine according to the present invention, the patient shows observable and/or measurable reduction in or absence of one or more of the following: reduction in the number of pathogenic cells; reduction in the percent of total cells that are pathogenic; and/or relief to some extent, one or more of the symptoms associated with the specific disease or condition; reduced morbidity and mortality, and improvement in quality of life issues. The above parameters for assessing successful treatment and improvement in the disease are readily measurable by routine procedures familiar to a physician.
“About” preceding a value means plus or less 10% of said value.

### DETAILED DESCRIPTION

1. Modified VSV-G

The present invention relates to a nucleic acid encoding a vesicular stomatitis virus glycoprotein (VSV-G) comprising at least one heterologous peptide. By “heterologous peptide” is meant a peptide which is not endogenous or native to a VSV-G protein, preferably to a VSV-G wild-type protein. Therefore, in one embodiment, the present invention relates to a nucleic acid encoding a modified vesicular stomatitis virus glycoprotein (VSV-G) comprising at least one heterologous peptide. In one embodiment, the nucleic acid of the heterologous peptide is inserted into the nucleic acid of VSV-G.

Within the meaning of the present invention, the term “modified VSV-G” amounts to the equivalent terms “chimeric VSV-G” and “mutant VSV-G”. All terms are used interchangeably throughout the present specification. In one embodiment, a chimeric VSV-G is a VSV-G comprising at least one heterologous peptide. In one embodiment, a mutant VSV-G is an insertion mutant, wherein at least one heterologous peptide is inserted into VSV-G. In one embodiment, the terms “modified”, “chimeric” and “mutant” are applied in reference to a VSV-G wild-type protein.

In one embodiment, the nucleic acid encoding a modified VSV-G of the invention is an isolated nucleic acid.

The present invention further relates to a modified vesicular stomatitis virus glycoprotein (VSV-G) comprising at least one heterologous peptide.

In one embodiment, the modified VSV-G of the invention is a recombinant modified VSV-G.

In one embodiment, the modified VSV-G of the invention is an isolated modified VSV-G.

1.1. VSV-G

Vesicular stomatitis viruses are constitutive members of the genus Vesiculovirus of the family Rhabdoviridae. Their genome accounts for a single molecule of negative-sense RNA, that encodes five major proteins: glycoprotein (G), polymerase or large protein (L), phosphoprotein (P), matrix protein (M) and nucleoprotein (N). The glycoprotein of the vesicular stomatitis virus (VSV-G) is a transmembrane protein that functions as the surface coat of the wild-type viral particles.

Presently, nine vesicular stomatitis virus (VSV) strains are classified in the Vesiculovirus genus: vesicular stomatitis Indiana virus (VSIV), vesicular stomatitis Alagoas virus (VSAV), Carajás virus (CJSV), Chandipura virus (CHPV), Cocal virus (COCV), Isfahan virus (ISFV), Maraba virus (MARAV), vesicular stomatitis New Jersey virus (VSNJV) and Piry virus (PIRYV). Additionally, other stains are provisionally classified in the Vesiculovirus genus: Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURV), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viraemia of carp virus (SVCV), Tupaia virus (TUPV), Ulcerative disease rhabdovirus (UDRV) and Yug Bogdanovac virus (YBV).

Among these strains, the protein G genes show sequence similarities. The VSV-G protein presents a N-terminal ectodomain, a transmembrane region and a C-terminal cytoplasmic tail. It is exported to the cell surface via the trans Golgi network (endoplasmic reticulum and Golgi apparatus).

Sequences alignments using MUSCLE (Multiple Sequence Comparison by Log-Expectation) are shown in Table 1 below.

TABLE 1
VSV-G sequence alignments using MUSCLE.

| | VSIV-G | VSNJV-G | CHPV-G | COCV-G | PIRYV-G | ISFV-G | SVCV-G | VSAV-G | CJSV-G | MARAV-G |
|---|---|---|---|---|---|---|---|---|---|---|
| | SEQ ID | SEQ ID | SEQ ID | SEQ ID | SEQ ID | SEQ ID | SEQ ID | SEQ ID | SEQ ID | SEQ ID |
| | NO: 1 | NO: 2 | NO: 3 | NO: 4 | NO: 5 | NO: 6 | NO: 7 | NO: 54 | NO: 55 | NO: 56 |
| VSIV-G | 100 | | | | | | | | | |
| SEQ ID NO: 1 | | | | | | | | | | |
| VSNJV-G | 51 | 100 | | | | | | | | |
| SEQ ID NO: 2 | | | | | | | | | | |
| CHPV-G | 41 | 38 | 100 | | | | | | | |
| SEQ ID NO: 3 | | | | | | | | | | |
| COCV-G | 71 | 49 | 41 | 100 | | | | | | |
| SEQ ID NO: 4 | | | | | | | | | | |
| PIRYV-G | 40 | 40 | 52 | 40 | 100 | | | | | |
| SEQ ID NO: 5 | | | | | | | | | | |
| ISFV-G | 41 | 40 | 55 | 42 | 51 | 100 | | | | |
| SEQ ID NO: 6 | | | | | | | | | | |
| SVCV-G | 32 | 32 | 32 | 32 | 32 | 31 | 100 | | | |
| SEQ ID NO: 7 | | | | | | | | | | |
| VSAV-G | 63 | 50 | 42 | 67 | 40 | 39 | 33 | 100 | | |
| SEQ ID NO: 54 | | | | | | | | | | |
| CJSV-G | 56 | 52 | 42 | 56 | 42 | 40 | 32 | 56 | 100 | |
| SEQ ID NO: 55 | | | | | | | | | | |
| MARAV-G | 78 | 51 | 42 | 74 | 41 | 40 | 35 | 65 | 56 | 100 |
| SEQ ID NO: 56 | | | | | | | | | | |

In one embodiment, the vesicular stomatitis virus glycoprotein (VSV-G) is VSV-G from VSIV (VSIV-G). In one embodiment, VSV-G from VSIV comprises or consists of SEQ ID NO: 1.

In one embodiment, VSV-G is a variant of SEQ ID NO: 1. In one embodiment, a variant of SEQ ID NO: 1 is a protein having a sequence identity of at least 30%, preferably of at least 35, 40, 45, 50, 55, 60, 65, 70, 75, 80, 85, 90, 95, 96, 97, 98, 99% or more with SEQ ID NO: 1.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Arthur M. Lesk, Computational Molecular Biology: Sources and Methods for Sequence Analysis (New-York: Oxford University Press, 1988); Douglas W. Smith, Biocomputing: Informatics and Genome Projects (New-York: Academic Press, 1993); Hugh G. Griffin and Annette M. Griffin, Computer Analysis of Sequence Data, Part 1 (New Jersey: Humana Press, 1994); Gunnar von Heinje, Sequence Analysis in Molecular Biology: Treasure Trove or Trivial Pursuit (Academic Press, 1987); Michael Gribskov and John Devereux, Sequence Analysis Primer (New York: M. Stockton Press, 1991); and Carillo et al., 1988. SIAM J. Appl. Math. 48(5):1073-1082. Preferred methods for determining identity are designed to give the largest match between the sequences tested. Methods of determining identity are described in publicly available computer programs. Preferred computer program methods for determining identity between two sequences include the GCG program package, including GAP (Devereux et al., 1984. Nucl. Acid. Res. 12(1 Pt 1):387-395; Genetics Computer Group, University of Wisconsin Biotechnology Center, Madison, Wis.), BLASTP, BLASTN, TBLASTN and FASTA (Altschul et al., 1990. J. Mol. Biol. 215(3):403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990. J. Mol. Biol. 215(3):403-410). The well-known Smith Waterman algorithm may also be used to determine identity.

In another embodiment, a variant of SEQ ID NO: 1 comprises conservative amino acid substitutions as compared to the sequence of SEQ ID NO: 1.

As used herein, the term “conservative amino acid substitution” is defined herein as an amino acid exchange within one of the following five groups:
I. Small aliphatic, nonpolar or slightly polar residues: Ala, Ser, Thr, Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met, Leu, Ile, Val, Cys;
V. Large, aromatic residues: Phe, Tyr, Trp.

As used herein, “amino acids” are represented by their full name, their three letter code or their one letter code as well known in the art. Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

As used herein, the term “amino acids” includes both natural and synthetic amino acids, and both D and L amino acids. “Standard amino acid” or “naturally occurring amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. For example, naphtlylalanine can be substituted for tryptophan to facilitate synthesis. Other synthetic amino acids that can be substituted include, but are not limited to, L-hydroxypropyl, L-3,4-dihydroxyphenylalanyl, α-amino acids such as L-α-hydroxylysyl and D-α-methylalanyl, L-α-methylalanyl, β-amino acids, and isoquinolyl.

As used herein, “amino acid” also encompasses chemically modified amino acids, including, but not limited to, salts, amino acid derivatives (such as amides), and substitutions. Amino acids contained within the polypeptides of the present invention, and particularly at the carboxy- or amino-terminus, can be modified by methylation, amidation, acetylation or substitution with other chemical groups which can change the polypeptide's circulating half-life without adversely affecting their activity. Additionally, a disulfide linkage may be present or absent in the polypeptides of the invention.

In another embodiment, a variant of SEQ ID NO: 1 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 1 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from VSNJV (VSNJV-G). In one embodiment, VSV-G from VSNJV comprises or consists of SEQ ID NO: 2.

In one embodiment, VSV-G is a variant of SEQ ID NO: 2. In one embodiment, a variant of SEQ ID NO: 2 is a protein having a sequence identity of at least 30%, preferably of at least 35, 40, 45, 50, 55, 60, 65, 70, 75, 80, 85, 90, 95, 96, 97, 98, 99% or more with SEQ ID NO: 2. In another embodiment, a variant of SEQ ID NO: 2 comprises conservative amino acid substitutions as compared to the sequence of SEQ ID NO: 2.

In another embodiment, a variant of SEQ ID NO: 2 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 2 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from CHPV (CHPV-G). In one embodiment, VSV-G from CHPV comprises or consists of SEQ ID NO: 3.

In one embodiment, VSV-G is a variant of SEQ ID NO: 3. In one embodiment, a variant of SEQ ID NO: 3 is a protein having a sequence identity of at least 30%, preferably of at least 35, 40, 45, 50, 55, 60, 65, 70, 75, 80, 85, 90, 95, 96, 97, 98, 99% or more with SEQ ID NO: 3. In another embodiment, a variant of SEQ ID NO: 3 comprises conservative amino acid substitutions as compared to the sequence of SEQ ID NO: 3.

In another embodiment, a variant of SEQ ID NO: 3 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 3 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from COCV (COCV-G). In one embodiment, VSV-G from COCV comprises or consists of SEQ ID NO: 4.

In one embodiment, VSV-G is a variant of SEQ ID NO: 4. In one embodiment, a variant of SEQ ID NO: 4 is a protein having a sequence identity of at least 30%, preferably of at least 35, 40, 45, 50, 55, 60, 65, 70, 75, 80, 85, 90, 95, 96, 97, 98, 99% or more with SEQ ID NO: 4. In another embodiment, a variant of SEQ ID NO: 4 comprises conservative amino acid substitutions as compared to the sequence of SEQ ID NO: 4.

In another embodiment, a variant of SEQ ID NO: 4 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 4 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from PIRYV (PIRYV-G). In one embodiment, VSV-G from PIRYV comprises or consists of SEQ ID NO: 5.

In one embodiment, VSV-G is a variant of SEQ ID NO: 5. In one embodiment, a variant of SEQ ID NO: 5 is a protein having a sequence identity of at least 30%, preferably of at least 35, 40, 45, 50, 55, 60, 65, 70, 75, 80, 85, 90, 95, 96, 97, 98, 99% or more with SEQ ID NO: 5. In another embodiment, a variant of SEQ ID NO: 5 comprises conservative amino acid substitutions as compared to the sequence of SEQ ID NO: 5.

In another embodiment, a variant of SEQ ID NO: 5 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 5 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from ISFV (ISFV-G). In one embodiment, VSV-G from ISFV comprises or consists of SEQ ID NO: 6.

In one embodiment, VSV-G is a variant of SEQ ID NO: 6. In one embodiment, a variant of SEQ ID NO: 6 is a protein having a sequence identity of at least 30%, preferably of at least 35, 40, 45, 50, 55, 60, 65, 70, 75, 80, 85, 90, 95, 96, 97, 98, 99% or more with SEQ ID NO: 6. In another embodiment, a variant of SEQ ID NO: 6 comprises conservative amino acid substitutions as compared to the sequence of SEQ ID NO: 6.

In another embodiment, a variant of SEQ ID NO: 6 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 6 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from SVCV (SVCV-G). In one embodiment, VSV-G from SVCV comprises or consists of SEQ ID NO: 7.

In one embodiment, VSV-G is a variant of SEQ ID NO: 7. In one embodiment, a variant of SEQ ID NO: 7 is a protein having a sequence identity of at least 30%, preferably of at least 35, 40, 45, 50, 55, 60, 65, 70, 75, 80, 85, 90, 95, 96, 97, 98, 99% or more with SEQ ID NO: 7. In another embodiment, a variant of SEQ ID NO: 7 comprises conservative amino acid substitutions as compared to the sequence of SEQ ID NO: 7.

In another embodiment, a variant of SEQ ID NO: 7 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 7 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from VSAV (VSAV-G). In one embodiment, VSV-G from VSAV comprises or consists of SEQ ID NO: 54.

In one embodiment, VSV-G is a variant of SEQ ID NO: 54. In one embodiment, a variant of SEQ ID NO: 54 is a protein having a sequence identity of at least 30%, preferably of at least 35, 40, 45, 50, 55, 60, 65, 70, 75, 80, 85, 90, 95, 96, 97, 98, 99% or more with SEQ ID NO: 54. In another embodiment, a variant of SEQ ID NO: 54 comprises conservative amino acid substitutions as compared to the sequence of SEQ ID NO: 54.

In another embodiment, a variant of SEQ ID NO: 54 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 54 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from CJSV (CJSV-G). In one embodiment, VSV-G from CJSV comprises or consists of SEQ ID NO: 55.

In one embodiment, VSV-G is a variant of SEQ ID NO: 55. In one embodiment, a variant of SEQ ID NO: 55 is a protein having a sequence identity of at least 30%, preferably of at least 35, 40, 45, 50, 55, 60, 65, 70, 75, 80, 85, 90, 95, 96, 97, 98, 99% or more with SEQ ID NO: 55. In another embodiment, a variant of SEQ ID NO: 55 comprises conservative amino acid substitutions as compared to the sequence of SEQ ID NO: 55.

In another embodiment, a variant of SEQ ID NO: 55 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 55 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

In one embodiment, the vesicular stomatitis virus glycoprotein (VSV-G) is VSV-G from MARAV (MARAV-G). In one embodiment, VSV-G from MARAV comprises or consists of SEQ ID NO: 56.

In one embodiment, VSV-G is a variant of SEQ ID NO: 56. In one embodiment, a variant of SEQ ID NO: 56 is a protein having a sequence identity of at least 30%, preferably of at least 35, 40, 45, 50, 55, 60, 65, 70, 75, 80, 85, 90, 95, 96, 97, 98, 99% or more with SEQ ID NO: 56. In another embodiment, a variant of SEQ ID NO: 56 comprises conservative amino acid substitutions as compared to the sequence of SEQ ID NO: 56.

In another embodiment, a variant of SEQ ID NO: 56 is a protein wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) from the sequence of SEQ ID NO: 56 is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24 or 25 amino acids (either contiguous or not) is/are added.

The modified VSV-G of the invention may comprise naturally standard amino acids or nonstandard amino acids. Polypeptide mimetics include polypeptides having the following modifications:
i) polypeptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;
ii) polypeptides wherein the N-terminus is derivatized to a —NRR¹ group, to a —NRC(O)R group, to a —NRC(O)OR group, to a —NRS(O)₂R group, to a —NHC(O)NHR group where R and R¹ are hydrogen or C₁-C₄ alkyl with the proviso that R and R¹ are not both hydrogen;
iii) polypeptides wherein the C terminus is derivatized to —C(O)R² where R² is selected from the group consisting of C₁-C₄ alkoxy and —NR³R⁴, where R³ and R⁴ are independently selected from the group consisting of hydrogen and C₁-C₄ alkyl.

In one embodiment of the invention, the modified VSV-G as described herein above are modified by means well-known in the art, for instance by the addition of one or more functional group such as a phosphate, acetate, lipid or carbohydrate group, and/or by the addition of one or more protecting group.

For example, the modified VSV-G can be modified by the addition of one or more functional groups such as phosphate, acetate, or various lipids and carbohydrates. The modified VSV-G of the invention can also exist as protein derivatives. The term “protein derivative” refers to compound having an amino group (—NH—), and more particularly, a peptide bond. Modified VSV-G may be regarded as substituted amides. Like the amide group, the peptide bond shows a high degree of resonance stabilization. The C—N single bond in the peptide linkage has typically about 40 percent double-bond character and the C═O double bond about 40 percent single-bond character. “Protecting groups” are those groups that prevent undesirable reactions (such as proteolysis) involving unprotected functional groups. Specific examples of amino protecting groups include formyl; trifluoroacetyl; benzyloxycarbonyl; substituted benzyloxycarbonyl such as (ortho- or para-) chlorobenzyloxycarbonyl and (ortho- or para-) bromobenzyloxycarbonyl; and aliphatic oxycarbonyl such as t-butoxycarbonyl and t-amiloxycarbonyl. The carboxyl groups of amino acids can be protected through conversion into ester groups. The ester groups include benzyl esters, substituted benzyl esters such as methoxybenzyl ester; alkyl esters such as cyclohexyl ester, cycloheptyl ester or t-butyl ester. The guanidino moiety may be protected by nitro; or arylsulfonyl such as tosyl, methoxybenzensulfonyl or mesitylenesulfonyl, even though it does not need a protecting group. The protecting groups of imidazole include tosy, benzyl and dinitrophenyl. The indole group of tryptophan may be protected by formyl or may not be protected.

In one embodiment, the modified VSV-G of the invention comprises a signal peptide at the N-terminus of said modified VSV-G. In one embodiment, the modified VSV-G of the invention comprises a signal peptide at the C-terminus of said modified VSV-G.

In one embodiment, the signal peptide comprises or consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues.

In one embodiment, the signal peptide of the modified VSV-G of the invention comprises or consists of SEQ ID NO: 52 (MKCLLYLAFLFIGVNC).

In another embodiment, the signal peptide of the modified VSV-G of the invention comprises or consists of the Gaussia princeps luciferase signal peptide with SEQ ID NO: 53 (MGVKVLFALICIAVAEA).

In another embodiment, the signal peptide of the modified VSV-G of the invention comprises of consists of any of the signal peptides disclosed in Kober et al., 2013. Biotechnol. Bioeng. 110:1164-1173; Mori et al., 2015. J. Biosci. Bioeng. 120(5):518-525; Stern et al., 2007. Trends Cell Mol. Bio. 2:1-17; Wen et al., 2011. Acta Biochim Biophys Sin. 43:96-102. These include, without limitation:
the signal peptide of the Mus musculus Ig kappa light chain precursor (mutant A2) comprising or consisting of SEQ ID NO: 57 (MDMRAPAGIFGFLLVLFPGYRS);
the signal peptide of the Homo sapiens serum albumin preproprotein comprising or consisting of SEQ ID NO: 58 (MKWVTFISLLFLFSSAYS);
the signal peptide of the Homo sapiens immunoglobulin heavy chain comprising or consisting of SEQ ID NO: 59 (MDWTWRVFCLLAVTPGAHP);
the signal peptide of the Homo sapiens immunoglobulin light chain comprising or consisting of SEQ ID NO: 60 (MAWSPLFLTLITHCAGSWA);
the signal peptide of the Homo sapiens azurocidin preproprotein comprising or consisting of SEQ ID NO: 61 (MTRLTVLALLAGLLASSRA);
the signal peptide of the Homo sapiens Cystatin-S precursor comprising or consisting of SEQ ID NO: 62 (MARPLCTLLLLMATLAGALA);
the signal peptide of the Pseudopleuronectes americanus trypsinogen 2 precursor comprising or consisting of SEQ ID NO: 63 (MRSLVFVLLIGAAFA);
the signal peptide of the Mesobuthus martensii potassium channel blocker comprising or consisting of SEQ ID NO: 64 (MSRLFVFILIALFLSAIIDVMS);
the signal peptide of the Conus leopardus α-conotoxin lp1.3 comprising or consisting of SEQ ID NO: 65 (MGMRMMFIMFMLVVLATTVVS);
the signal peptide of the Saccharomyces cerevisiae α-galactosidase (mutant m3) comprising or consisting of SEQ ID NO: 66 (MRAFLFLTACISLPGVFG);
the signal peptide of the Aspergillus niger cellulase comprising or consisting of SEQ ID NO: 67 (MKFQSTLLLAAAAGSALA);
the signal peptide of the Nepenthes gracilis aspartic proteinase nepenthesin-1 comprising or consisting of SEQ ID NO: 68 (MASSLYSFLLALSIVYIFVAPTHS);
the signal peptide of the Nepenthes rafflesiana acid chitinase comprising or consisting of SEQ ID NO: 69 (MKTHYSSAILPILTLFVFLSINPSHG);
the signal peptide of the M28 virus K28 prepro-toxin comprising or consisting of SEQ ID NO: 70 (MESVSSLFNIFSTIMVNYKSLVLALLSVSNLKYARG);
the signal peptide of the Zygosaccharomyces bailii killer toxin zygocin precursor comprising or consisting of SEQ ID NO: 71 (MKAAQILTASIVSLLPIYTSA);
the signal peptide of the Vibrio cholerae O139 cholera toxin comprising or consisting of SEQ ID NO: 72 (MIKLKFGVFFTVLLSSAYA);
the signal peptide of the Saccharomyces cerevisiae-derived adhesion subunit of α-agglutinin comprising or consisting of SEQ ID NO: 73 (MQLLRCFSIFSVIASVLAQELTTICEQIPSPTLESTPYSLSTTTILANGK);
the signal peptide of the Saccharomyces cerevisiae-derived exo-1,3-β glucanase comprising or consisting of SEQ ID NO: 74 (MLSLKTLLCTLLTVSSVLATPVPARDPSSIQFVHEENKKRYYDYDHGSLGE);
the signal peptide of the Saccharomyces cerevisiae-derived mating pheromone α-factor comprising or consisting of SEQ ID NO: 75 (MRFPSIFTAVLFAASSALAAPVNTTTEDETAQIPAEAVIGYLDLEGDFDVAVLPFS NSTNN);
the signal peptide of the Saccharomyces cerevisiae-derived chitin trans-glycosylase comprising or consisting of SEQ ID NO: 76 (MKVLDLLTVLSASSLLSTFAAAESTATADSTTAASSTASCNPLKTTGCTPDTALA TSFSEDFSSSSK);
the signal peptide of the Saccharomyces cerevisiae-derived phospholipase B comprising or consisting of SEQ ID NO: 77 (MKLQSLLVSAAVLTSLTENVNAWSPNNSYVPANVTCDDDINLVREASGLSDNET EWLKKRDAYTKE);
the signal peptide of the Saccharomyces cerevisiae-derived cell wall protein related to glucanases comprising or consisting of SEQ ID NO: 78 (MKLSATTLTAASLIGYSTIVSALPYAADIDTGCTTTAHGSHQHKRAVAVTYVYET VTVDKNGQTVTPTSTEASSTVASTTTLISESSVTKSSSKVASSSE);
the signal peptide of the Saccharomyces cerevisiae-derived phospholipase B comprising or consisting of SEQ ID NO: 79 (MQLRNILQASSLISGLSLAADSSSTTGDGYAPSIIPCPSDDTSLVRNASGLSTAETD WLKKRDAYTKEALHSFLSRATSNFSDTSLLSTLFSSNSSN);
the signal peptide of the Saccharomyces cerevisiae-derived exo-1,3-β glucanase comprising or consisting of SEQ ID NO: 80 (MISPISFLSSLLCLTYLTSALPILPKREVVTRVHTASTTNVVTDFYSTTTE);
the signal peptide of the Saccharomyces cerevisiae-derived cell wall-associated protein involved in export of acetylated sterols comprising or consisting of SEQ ID NO: 81 (MLEFPISVLLGCLVAVKAQTTFPNFESDVLNEHNKFRALHVDTAP LTWSDTLATYAQNYADQYDCSGVLTHSDGPYGENLALGYTDTGAVDAWYGEIS KY);
the signal peptide of the Saccharomyces cerevisiae-derived aspartic protease comprising or consisting of SEQ ID NO: 82 (MKLKTVRSAVLSSLFASQVLGKIIPAANKRDDDSNSKFVKLPFHKLYGDSLENVG SDKKPEVRLLKRADGYEEIIITNQQSFYSVDLE);
the signal peptide of the Saccharomyces cerevisiae-derived cell wall mannoprotein comprising or consisting of SEQ ID NO: 83 (MVKLTSIAAGVAAIAATASATTTLAQSDERVNLVELGVYVSDIRAHLAQYYSFQ VAHPTETY);
the signal peptide of the Saccharomyces cerevisiae-derived cell wall mannoprotein comprising or consisting of SEQ ID NO: 84 (MVKLTSIVAGVAAIAAGVAAAPATTTLSPSDERVNLVELGVYVSDIRAHLAEYY MFQAAHPTETY);
the signal peptide of the Saccharomyces cerevisiae-derived mating pheromone α-factor comprising or consisting of SEQ ID NO: 85 (MQPITTASTQATQKDKSSEKKDNYIIKGLFWDPACVIA);
the signal peptide of the Saccharomyces cerevisiae-derived sporulation-specific exo-1,3-b-glucanase comprising or consisting of SEQ ID NO: 86 (MVSFRGLTTLTLLFTKLVNCNPVSTKNRDSIQFIYKEKDSIYSAINNQAINEK);
the signal peptide of the Homo sapiens chymotrypsinogen comprising or consisting of SEQ ID NO: 87 (MAFLWLLSCWALLGTTFG);
the signal peptide of the Homo sapiens interleukin-2 comprising or consisting of SEQ ID NO: 88 (MQLLSCIALILALV);
the signal peptide of the Homo sapiens trypsinogen-2 comprising or consisting of SEQ ID NO: 89 (MNLLLILTFVAAAVA);
the signal peptide of the Metridia longa luciferase comprising or consisting of SEQ ID NO: 90 (MDIKVVFTLVFSALVQA);
the signal peptide of the Oikopleura dioica Oikosin 1 comprising or consisting of SEQ ID NO: 91 (MLLLSALLLGLAHGYS);
the signal peptide of the Oikopleura dioica Oikosin 2A comprising or consisting of SEQ ID NO: 92 (MKLLASVLTIAAADYACC);
the signal peptide of the Oikopleura dioica Oikosin 3 comprising or consisting of SEQ ID NO: 93 (MKISAGLLGVALGQNEGSAEA);
the signal peptide of the Oikopleura dioica Oikosin 4A comprising or consisting of SEQ ID NO: 94 (MKLFAALSAFSASVEA);
the signal peptide of the Oikopleura dioica Oikosin 5A comprising or consisting of SEQ ID NO: 95 (MKLLCSVLLGTVFG);
the signal peptide of the Oikopleura dioica Oikosin 6A comprising or consisting of SEQ ID NO: 96 (MKISPLLVVTAVVG);
the signal peptide of the Oikopleura dioica Oikosin 7A comprising or consisting of SEQ ID NO: 97 (MKIAATFAALASATEWQG);
the signal peptide of the Vargula hilgendorfii luciferase comprising or consisting of SEQ ID NO: 98 (MKIIILSVILAYCVTDNC);
the signal peptide of the Methanococcus jannaschii Slmj1 comprising or consisting of SEQ ID NO: 99 (MAMSLKKIGAIAVGGAMVATALASGVAA);
the signal peptide of the Hepatitis C virus serotype 1b E1 protein comprising or consisting of SEQ ID NO: 100 (MGCSFSIFLLALLSCLTTPASA);
the signal peptide of the Hepatitis C virus serotype 1b E2 protein comprising or consisting of SEQ ID NO: 101 (MVGNWAKVLIVMLLFAGVDG);
the signal peptide of the tissue plasminogen activator comprising or consisting of SEQ ID NO: 102 (MDAMKRGLCCVLLLCGAVFVDSVTG); and
the signal peptide comprising or consisting of SEQ ID NO: 103 (MDAMKVLLLVFVSPSQVTG).

1.2. Peptide

1.2.1. Antigen

In one embodiment, the at least one heterologous peptide of the invention is an antigen or a fragment thereof. In one embodiment, a fragment of an antigen is an epitope.

In one embodiment, the antigen is a non-self antigen, i.e., the antigen is a foreign antigen. In another embodiment, the antigen is a protein of the host, i.e., is a self-antigen.

By “non-self antigen”, “heterologous antigen” or “foreign antigen” is meant a molecule or molecules which is/are not endogenous or native to a subject which is exposed to it. The foreign antigen may elicit an immune response, e.g., a humoral and/or T cell mediated response in the mammal.

Examples of foreign antigen include, but are not limited to, proteins (including a modified protein such as a glycoprotein, a mucoprotein, etc.), nucleic acids, carbohydrates, proteoglycans, lipids, mucin molecules, immunogenic therapeutic agents (including proteins such as antibodies, particularly antibodies comprising non-human amino acid residues, e.g., rodent, chimeric/humanized, and primatized antibodies), toxins (optionally conjugated to a targeting molecule such as an antibody, wherein the targeting molecule may also be immunogenic), gene therapy viral vectors (such as retroviruses and adenoviruses), grafts (including antigenic components of the graft to be transplanted into the heart, lung, liver, pancreas, kidney of graft recipient and neural graft components), infectious agents (such as bacteria and virus or other organism, e.g., protists), alloantigens (i.e., an antigen that occurs in some, but not in other members of the same species) such as differences in blood types, human lymphocyte antigens (HLA), platelet antigens, antigens expressed on transplanted organs, blood components, pregnancy (Rh), and hemophilic factors (e.g., Factor VTfl and Factor IX).

By “self-antigen” is meant an antigen that is normally expressed in a body. In one embodiment, self-antigen is expressed in an organ that is the target of an autoimmune disease. In one embodiment, the self-antigen is expressed in a pancreas, thyroid, connective tissue, kidney, lung, digestive system or nervous system. In another embodiment, self-antigen is expressed on pancreatic β cells.

Examples of self-antigen include, but are not limited to, antigenic peptides of insulin, insulin β, glutamic acid decarboxylase 1 (GAD1), glutamic acid decarboxylase 65 (GAD 65), HSP, thyroglobulin, nuclear proteins, acetylcholine receptor, collagen, thyroid stimulating hormone receptor (TSHR), ICA512(IA-2) and IA-2β (phogrin), carboxypeptidase H, ICA69, ICA12, thyroid peroxidase, native DNA, myelin basic protein, myelin proteolipid protein, acetylcholine receptor components, histocompatibility antigens, antigens involved in graft rejection and altered peptide ligands.

1.2.1.1. Tumor Antigens

In one embodiment, the antigen is a tumor antigen, or tumor-associated antigen.

In one embodiment, the antigen is a tumor-specific antigen (TSA). In another embodiment, the antigen is a tumor-associated antigen (TAA). In another embodiment, the antigen is a cancer-germline/cancer testis antigen (CTA).

In one embodiment, the tumor from which the antigen is isolated or derived is any tumor or cancer, including, but not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof.

In one embodiment, the antigen may be any tumor antigen known from the person skilled in the art. For example, the antigen is selected from the tumor T cell antigen database TANTIGEN (http://cvc.dfci.harvard.edu/tadb/index.html).

Examples of tumor antigens comprise those described in Table 3 of Cheever et al., 2009. Clin Cancer Res. 15(17):5323-37, including, but not limited to, WT1, MUC1, LMP2, HPV E6 E7, EGFRvIII, HER-2/neu, Idiotype, MAGE A3, p53 nonmutant, NY-ESO-1, PSMA, GD2, CEA, Melan-A/MART1, Ras mutant, gp100, p53 mutant, Proteinase3 (PR1), bcr-abl, Tyrosinase, Survivin, PSA, hTERT, Sarcoma translocation breakpoints, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β. MAD-CT-2 and Fos-related antigen 1.

Further examples of tumor antigens include, but are not limited to, 707-AP (707 alanine proline), AFP (α-fetoprotein), ART-4 (adenocarcinoma antigen recognized by T cells), BAGE (B antigen, β-catenin/m, β-catenin/mutated), Bcr-abl (breakpoint clusterregion-Abelson), CA-125 (cancer antigen 125, carcinoma antigen 125, or carbohydrate antigen 125, also known as mucin 16 or MUC16), CAMEL (CTL-recognized antigen on melanoma), CAP-1 (carcinoembryonic antigen peptide-1), CASP-8 (caspase-8), CDC27m (cell-division-cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma (the epitopes of DAM-6 and DAM-10 are equivalent, but the gene sequences are different. DAM-6 is also called MAGE-B2 and DAM-10 is also called MAGE-B1)), EGF-R, ELF2M (elongation factor 2 mutated), ETA (Epithelial Tumor Antigen), ETV6-AML1 (Ets variant gene 6/acute myeloid leukemia 1 gene ETS), G250 (glycoprotein 250), GAGE (G antigen), GnT-V (N-acetylglucosaminyltransferase V), Gp100 (glycoprotein 100 kD), HAGE (helicose antigen), HER-2/neu (human epidermal receptor-2/neurological), HLA-A*0201-R170I (arginine (R) to isoleucine (I) exchange at residue 170 of the α-helix of the α2-domain in the HLA-A2 gene), HPV-E6 (human papilloma virus E6), HPV-E7 (human papilloma virus E7), HSP70-2M (heat shock protein 70-2 mutated), HST-2 (human signet ring tumor-2), hTERT or hTRT (human telomerase reverse transcriptase), iCE (intestinal carboxylesterase), KIAA0205 (name of the gene as it appears in databases), LAGE (L antigen), LDLR/FUT (low density lipid receptor/GDP-L-fucose: β-D-galactosidase 2-α-L-fucosyltransferase), MAGE (melanoma antigen, including but not limited to, MAGE3, MAGEA6, MAGEA10), MART-1/Melan-A (melanomaantigen recognized by T cells-1/Melanoma antigen A), MC1R (melanocortin 1 receptor), Myosin/m (myosin mutated), MUC1 (mucin 1), MUM-1, -2, -3 (melanomaubiquitous mutated 1, 2, 3), NA88-A (NA cDNA clone of patient M88), NY-ESO-1 (New York—esophageous 1), P1A, P15 (protein 15), p190 minor bcr-abl (protein of 190KD bcr-abl), Pml/RARα (promyelocytic leukaemia/retinoic acid receptor α), PRAME (preferentially expressed antigen of melanoma), PSA (prostate-specific antigen), PSMA (prostate-specific membrane antigen), RAGE (renal antigen), RU1 or RU2 (renalubiquitous 1 or 2), SAGE (sarcoma antigen), SART-1 or SART-3 (squamous antigen rejecting tumor 1 or 3), TEL/AML1 (translocation Ets-family leukemia/acute myeloidleukemia 1), TPI/m (triosephosphate isomerase mutated), tyrosinase, TRP-1 (tyrosinase related protein 1, or gp75), TRP-2 (tyrosinase related protein 2), TRP-2/INT2 (TRP-2/intron2), WT1 (Wilms' tumor gene), and mutant oncogenic forms of p53 (TP53), p73, ras, BRAF, APC (adenomatous polyposis coli), myc, VHL (von Hippel's Lindau protein), Rb-1 (retinoblastoma), Rb-2, BRCA1, BRCA2, AR (androgen receptor), Smad4, MDR1, Flt-3.

In a preferred embodiment, the antigen of the invention is selected from the group consisting of P1A, TRP-2, gp100, MART-1/Melan-A, tyrosinase, MAGE (including, but not limited to, MAGE3, MAGEA6, MAGEA10), NY-ESO-1, EGF-R, PSA, PSMA, CEA, HER2/neu, Muc-1, hTERT, TRP-1, BCR-abl, and mutant oncogenic forms of p53 (TP53), p73, ras, BRAF, APC (adenomatous polyposis coli), myc, VHL (von Hippel's Lindau protein), Rb-1 (retinoblastoma), Rb-2, BRCA1, BRCA2, AR (androgen receptor), Smad4, MDR1 and Flt-3.

According to the present invention, tumor antigens include any tumor antigen as described above, in addition to any other antigen that is associated with the risk of acquiring or development of cancer or for which an immune response against such antigen can have a therapeutic benefit against a cancer. For example, a cancer antigen could include, but is not limited to, a tumor antigen, a mammalian cell molecule harboring one or more mutated amino acids, a protein normally expressed pre- or neo-natally by mammalian cells, a protein whose expression is induced by insertion of an epidemiologic agent (e.g., virus), a protein whose expression is induced by gene translocation, and a protein whose expression is induced by mutation of regulatory sequences. Some of these antigens may also serve as antigens in other types of diseases (e.g., autoimmune disease).

1.2.1.2. Neoantigens

In another embodiment, the antigen of the invention is a neoantigen.

As used herein, the term “neoantigen” is a newly formed antigen that has not been previously recognized by the immune system. Neoantigens and, by extension, neoantigenic determinants (or neoepitopes), can be formed when a protein undergoes further modification within a biochemical pathway such as glycosylation, phosphorylation or proteolysis.

Neoantigens, tumor-specific or “somatic” mutations may be identified by comparing DNA isolated from tumor versus normal sources.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLiD system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention.

1.2.1.3. Pathogen Antigens

In one aspect of the invention, the antigen of the present invention is an antigen from a pathogen (including the whole pathogen). In a particular embodiment, the antigen is from a pathogen that is associated with (e.g., causes or contributes to) an infectious disease.

In one embodiment, the antigen of the invention is an infectious disease antigen.

In one embodiment, antigens from an infectious disease pathogen include antigens having epitopes that are recognized by T cells, antigens having epitopes that are recognized by B cells, antigens that are exclusively expressed by pathogens, and antigens that are expressed by pathogens and by other cells.

In one embodiment, pathogen antigens include whole cells and the entire pathogen organism, as well as lysates, extracts or other fractions thereof. In some embodiments, the antigens include organisms or portions thereof which may not be ordinarily considered to be pathogenic in a subject, but against which immunization is nonetheless desired.

In one embodiment, antigens include one, two or a plurality of antigens that are representative of the substantially all of the antigens present in the infectious disease pathogen against which the vaccine is to be administered. In other embodiments, antigens from two or more different strains of the same pathogen or from different pathogens can be used to increase the therapeutic efficacy and/or efficiency of the vaccine.

Pathogen antigens include, but are not limited to, antigens that are expressed by a bacterium, a virus, a parasite or a fungus.

In a particular embodiment, pathogen antigens of the present invention include antigens which cause a chronic infectious disease in an animal. In one embodiment, a pathogen antigen of the present invention includes an antigen from a virus.

Examples of viral antigens include, but are not limited to, env, gag, rev, tar, tat, nucleocapsid proteins and reverse transcriptase from immunodeficiency viruses (e.g., HIV, FIV); HBV surface antigen and core antigen; HCV antigens; influenza nucleocapsid proteins; parainfluenza nucleocapsid proteins; human papilloma type 16 E6 and E7 proteins; Epstein-Barr virus LMP-1, LMP-2 and EBNA-2; herpes LAA and glycoprotein D; as well as similar proteins from other viruses. Particularly preferred antigens for use in the present invention include, but are not limited to, HIV-1 gag, HIV-1 env, HIV-1 pol, HIV-1 tat, HIV-1 nef, HbsAG, HbcAg, hepatitis c core antigen, HPV E6 and E7, HSV glycoprotein D, and Bacillus anthracis protective antigen.

Examples of bacterial antigens include, but are not limited to, Borrelia afzelii antigens, Borrelia garinii antigens, Brucella abortus antigens, Campylobacter jejuni antigens, Helicobacter pylori antigens, Legionella pneumophila antigens, Leptospira biflexa antigens, Mycoplasma pneumoniae antigens, Yersinia enterocolitica antigens, Chlamydia pneumoniae antigens, Chlamydia trachomatis antigens, Chlamydia abortus antigens, Clostridium difficile antigens, Neisseria gonorrhoeae antigens, Toxoplasma gondii antigens, Bordetella pertussis Filamentous Hemagglutinin (FHA), and Bordetella pertussis toxin (Pertussis Toxin, PT).

Examples of fungi and parasitic antigens include, but are not limited to, Aspergillus fumigatus antigens and Candida albicans antigens.

In another embodiment, the antigen of the invention is capable of suppressing an undesired, or harmful, immune response. In one embodiment, the immune response is caused by allergens, autoimmune antigens, inflammatory agents, antigens involved in GVHD, certain cancers, septic shock antigens, and antigens involved in transplantation rejection. Such compounds include, but are not limited to, antihistamines, cyclosporin, corticosteroids, FK506, peptides corresponding to T cell receptors involved in the production of a harmful immune response, Fas ligands (i.e., compounds that bind to the extracellular or the cytosolic domain of cellular Fas receptors, thereby inducing apoptosis), suitable MHC complexes presented in such a way as to effect tolerization or anergy, T cell receptors, and autoimmune antigens, preferably in combination with a biological response modifier capable of enhancing or suppressing cellular and/or humoral immunity.

Other antigens useful in the present invention and combinations of antigens will be apparent to those of skill in the art. The present invention is not restricted to the use of the antigens as described above.

1.2.2. Epitope

In one embodiment, the at least one heterologous peptide of the invention is an epitope derived from an antigen as described hereinabove. Accordingly, in one embodiment, a fragment of antigen of the invention comprises or consists of an epitope or “antigen epitopic fragment”. In one embodiment, a fragment of antigen of the invention comprises or consists of more than one, i.e., at least two, three, four, five or more epitopes or “antigen epitopic fragments”.

In one embodiment, the epitope may be any epitope known from the person skilled in the art. For example, the epitope is selected from the immune epitope database and analysis resource (Vita et al., 2014. Nucleic Acids Res. 43(Database issue):D405-12; http://www.iedb.org).

In one embodiment, the epitope is derived from a non-self antigen or foreign antigen as described herein above. In another embodiment, the epitope is derived from a protein of the host, i.e., the epitope is derived from a self-antigen as described herein above.

In another embodiment, the epitope is derived from a neoantigen as described hereinabove, i.e., the epitope is a neoantigenic determinant.

In one embodiment, the epitope is a conformational epitope, i.e., is composed of discontinuous sections of the antigen's amino acid sequence. In another embodiment, the epitope is a linear epitope, i.e., is composed of a continuous section of the antigen's amino acid sequence.

1.2.2.1. T Cell Epitopes

In one embodiment, the epitope is a T cell epitope.

1.2.2.1.1. CD8 T Cell Epitopes

In one embodiment, the T cell epitope is a T cell epitope presented by MHC class I molecules. In one embodiment, the epitope is a CD8 T cell epitope.

Examples of CD8 T cell epitopes include, but are not limited to epitopes from, ovalbumin (with SEQ ID NO: 11), P1A (with SEQ ID NO: 13), MART-1 (with SEQ ID NO: 14), gp100 (with SEQ ID NO: 15), tyrosinase (with SEQ ID NO: 16), gp70 (with SEQ ID NO: 133) and TRP2 (with SEQ ID NO: 134).

1.2.2.1.2. CD4 T Cell Epitopes

In one embodiment, the T cell epitope is a T cell epitope presented by MHC class II molecules. In one embodiment, the epitope is a CD4 T cell epitope (or helper T cell epitope).

Examples of CD4 T cell epitopes include, but are not limited to epitopes from, ovalbumin (e.g., with SEQ ID NO: 12), pan HLA DR-binding epitope (PADRE) (e.g., with SEQ ID NO: 17), VIL1 (e.g., with SEQ ID NO: 18), tetanus toxoid epitope (TT) (e.g., with SEQ ID NO: 19), gp100 (e.g., with SEQ ID NO: 20), HMGB1-derived immunostimulatory peptide hp91 (e.g., with SEQ ID NO: 21) and NY-ESO-1 (e.g., with SEQ ID NO: 143).

Further examples of CD4 T cell epitopes include those disclosed in Hiemstra et al., Proc Natl Acad Sci USA. 1997 Sep. 16; 94(19): 10313-10318.

A limiting factor for targeting a specific CD4 response is the large number of polymorphisms in MHC class II genes. Therefore, in one embodiment, the CD4 T cell epitope may be a universal antigenic CD4 T cell epitope. As used herein, the term “universal antigenic CD4 T cell epitope” refers to an epitope whose amino acid sequence is derived from at least one universal antigenic (or universal immunogenic or broad range) CD4 T cell epitope (also called an immunogenic carrier peptide), which can be presented by multiple major histocompatibility complex (MHC) haplotypes and thereby activate helper CD4 T cells, which in turn, stimulate B cell growth and differentiation.

Examples of universal antigenic CD4 T cell epitopes include, but are not limited to, pan HLA DR-binding epitope (PADRE) (e.g., with SEQ ID NO: 17), natural tetanus sequences, epitopes derived from tetanus toxoid (TT) (e.g., with SEQ ID NO: 19) or diphtheria toxoid (DT), VIL1 (e.g., with SEQ ID NO: 18), HMGB1-derived immunostimulatory peptide hp91 (e.g., with SEQ ID NO: 21), NY-ESO-1 (e.g., with SEQ ID NO: 143), supermotif peptides from HIV-1 (Gag 171, Gag 294, Gag 298, Pol 303, Pol 335, Pol 596, Pol 711, Pol 712, Pol 758, Pol 915, Pol 956), and epitopes from hemagglutinin influenza virus protein.

In another embodiment, the CD4 T cell epitope may be a foreign CD4 T cell epitope, i.e., a foreign T cell epitope which binds an MHC class II molecule and can be presented on the surface of an antigen presenting cell (APC) bound to the MHC class II molecule.

1.2.2.1.3. Tumoral Epitopes

In one embodiment, the epitope is able to induce an immune response against tumor antigens. Accordingly, in one embodiment, the epitope is a tumoral epitope, preferably, the epitope is a tumoral CD4 T cell epitope or a tumoral CD8 T cell epitope. In one embodiment, the tumoral T cell epitope is a tumoral T cell epitope presented by MHC class I molecules. In another embodiment, the tumoral T cell epitope is a tumoral T cell epitope presented by MHC class II molecules.

Examples of tumoral T cell epitopes comprise those described in Vigneron et al., 2013. Cancer Immun. 13:15, including, but not limited to, those recited in Table 2 below:

TABLE 2
Examples of tumoral T cell epitopes.

| SEQ ID NO: | Epitope sequence | Antigen |
|---|---|---|
| 104 | ILDKVLVHL | CLPP |
| 105 | FLDRFLSCM | Cyclin-A1 |
| 106 | SLIAAAAFCLA | Cyclin-A1 |
| 107 | KEADPTGHSY | MAGE-A1 |
| 108 | RVRFFFPSL | MAGE-A1 |
| 109 | ILFGISLREV | MAGE-C1 |
| 110 | KVVEFLAML | MAGE-C1 |
| 111 | ASSTLYLVF | MAGE-C2 |
| 112 | SSTLYLVFSPSSFST | MAGE-C2 |
| 113 | FGRLQGISPKI | SSX2 |
| 114 | RQKKIRIQL | XAGE1b/GAGED2a |
| 115 | HLGSRQKKIRIQLRSQ | XAGE1b/GAGED2a |
| 116 | YTTAEEAAGIGILTVI | Melan-A/MART-1 |
| | LGVLLLIGCWYCRR | |
| 117 | SQWRVVCDSLEDYDT | TRP-1 |
| 118 | IYMDGTADFSF | Tyrosinase |
| 119 | KFLDALISL | CD45 |
| 120 | FVGEFFTDV | glypican-3 |
| 121 | EYILSLEEL | glypican-3 |
| 122 | NLSSAEVVV | IGF2B3 |
| 123 | RLLVPTQFV | IGF2B3 |
| 124 | FLGYLILGV | Kallikrein 4 |
| 125 | LLSDDDVVV | KIF20A |
| 126 | AQPDTAPLPV | KIF20A |
| 127 | CIAEQYHTV | KIF20A |
| 128 | FLPEFGISSA | Lengsin |
| 129 | RISSTLNDECWPA | Meloa |
| 130 | CPPWHPSERISSTL | Meloa |
| 131 | TCQPTCRSL | MUC5AC |
| 132 | TLGEFLKLDRERAKN | survivin |

1.2.2.1.4. Pathogenic Epitopes

In one embodiment, the epitope is able to induce an immune response against pathogenic antigens. In one embodiment, the epitope is a pathogenic epitope; preferably, the epitope is a pathogenic T cell epitope; more preferably, the epitope is a CD4 T cell epitope or a pathogenic CD8 T cell epitope.

In one embodiment, the pathogenic T cell epitope is a pathogenic T cell epitope presented by MHC class I molecules. In another embodiment, the pathogenic T cell epitope is a pathogenic T cell epitope presented by MHC class II molecules. In one embodiment, the epitope is a bacterial T cell epitope, a viral T cell epitope, a parasitic T cell epitope or a fungal T cell epitope.

Examples of pathogenic T cell epitopes comprise, but are not limited to, listeriolysin O protein of Listeria monocytogenes (e.g., with SEQ ID NO: 144), Influenza Virus Nucleoprotein (e.g., with SEQ ID NO: 145), lymphocytic choriomeningitis virus (LCMV) glycoprotein (GP) (e.g., with SEQ ID NO: 146 or 147) and immunodominant adeno-associated virus 2 (AAV2) (e.g., with SEQ ID NO: 148).

In one embodiment, the pathogenic T cell epitope is a HIV T cell epitope. Examples of HIV T cell epitopes include, without limitation, those discloses on Hiv.lanl.gov. (2017). HIV Molecular Immunology Database. [online] Available at: https://www.hiv.lanl.gov/content/immunology/index.html and in Yusim K, Korber B T M, Brander C, Barouch D, De Boer R, Haynes B F, Koup R, Moore J P, Walker B D and Watkins D I (Eds.). (2017). HIV Molecular Immunology 2016. Los Alamos, N. Mex.: Los Alamos National Laboratory, Theoretical Biology and Biophysics.

In one embodiment, the pathogenic T cell epitope is a hepatitis virus T cell epitope, including without limitation, hepatitis A virus (HAV), hepatitis B virus (HBV), hepatitis C virus (HCV), hepatitis D virus (HDV), hepatitis E virus (HEV), hepatitis F virus (HFV) or hepatitis G virus (HGV).

1.2.2.1. Long CD4/CD8 Epitopes

In one embodiment, the fragment of antigen according to the present invention comprises at least two epitopes.

In one embodiment, the fragment of antigen according to the present invention comprises at least two T cell epitopes, both presented by MHC class I molecules. In one embodiment, the fragment of antigen according to the present invention comprises at least two CD8 T cell epitopes.

In one embodiment, the fragment of antigen according to the present invention comprises at least two T cell epitopes, both presented by MHC class II molecules. In one embodiment, the fragment of antigen according to the present invention comprises at least two CD4 T cell epitopes.

In a preferred embodiment, the fragment of antigen according to the present invention comprises at least two T cell epitopes, at least one of which is presented by MHC class I molecules and at least one of which is presented by MHC class II molecules. In one embodiment, the fragment of antigen according to the present invention comprises at least two T cell epitopes, at least one of which is a CD4 T cell epitope and at least one of which is a CD8 T cell epitope.

Examples of fragments of antigen comprising at least two T cell epitopes include, but are not limited to, gp100 (with SEQ ID NO: 22) and P1A (with SEQ ID NO: 23).

In one embodiment, the fragment of antigen according to the present invention comprises more than two epitopes. In one embodiment, the fragment of antigen according to the present invention comprises 3, 4, 5, 6, 7, 8, 9, 10 or more epitopes.

1.2.2.2. Two or More Epitopes/VSV-G

In one embodiment, the modified VSV-G of the present invention comprises more than one heterologous peptide. In a particular embodiment, the modified VSV-G of the present invention comprises 2, 3, 4 or more heterologous peptides. In one embodiment, the modified VSV-G of the present invention comprises a combination of heterologous peptides.

In a particular embodiment, the modified VSV-G of the present invention comprises at least two heterologous peptides. In a preferred embodiment, the modified VSV-G of the present invention comprises at least two fragments of antigens. In a preferred embodiment, the modified VSV-G of the present invention comprises at least two epitopes. In one embodiment, the at least two heterologous peptides, preferably the at least two fragments of antigens or the at least two epitopes, are identical, i.e., the share the same amino acid sequence. In another embodiment, the at least two heterologous peptides, preferably the at least two fragments of antigens or at least two epitopes, are different, i.e., they don't share the same amino acid sequence.

In a more preferred embodiment, the modified VSV-G of the present invention comprises at least one CD8 T cell epitope and at least another epitope. In a more preferred embodiment, the modified VSV-G of the present invention comprises at least one CD4 T cell epitope and at least another epitope. In an even more preferred embodiment, the modified VSV-G of the present invention comprises at least one CD8 T cell epitope and at least one CD4 T cell epitope. In an even more preferred embodiment, the modified VSV-G of the present invention comprises at least two CD4 T cell epitopes, which may be identical or different, as defined hereinabove. In an even more preferred embodiment, the modified VSV-G of the present invention comprises at least two CD8 T cell epitopes, which may be identical or different, as defined hereinabove.

In one embodiment, the modified VSV-G of the present invention comprises at least one antigen or epitopic fragment thereof, preferably an epitope, and at least one CD4 T cell epitope.

In a preferred embodiment, the modified VSV-G of the present invention comprises at least one epitope, preferably a T cell epitope, and at least one CD4 T cell epitope, preferably a universal antigenic CD4 T cell epitope.

1.2.3. Length

1.2.3.1. Global

In one embodiment, the heterologous peptide or fragment thereof has a length of 4 to 50 amino acids, preferably 5 to 25 amino acids, more preferably 6 to 20 amino acids, even more preferably 8 to 18 amino acids.

In one embodiment, the heterologous peptide or fragment thereof has a length of 4 to 10 amino acids, 4 to 15 amino acids, 4 to 20 amino acids, 4 to 25 amino acids or 4 to 30 amino acids.

In another embodiment, the heterologous peptide or fragment thereof has a length of 5 to 10 amino acids, 5 to 15 amino acids, 5 to 20 amino acids, 5 to 25 amino acids or 5 to 30 amino acids.

In another embodiment, the heterologous peptide or fragment thereof has a length of 6 to 10 amino acids, 6 to 15 amino acids, 6 to 20 amino acids, 6 to 25 amino acids or 6 to 30 amino acids.

In another embodiment, the heterologous peptide or fragment thereof has a length of 7 to 10 amino acids, 7 to 15 amino acids, 7 to 20 amino acids, 7 to 25 amino acids or 7 to 30 amino acids.

In another embodiment, the heterologous peptide or fragment thereof has a length of 8 to 10 amino acids, 8 to 15 amino acids, 8 to 20 amino acids, 8 to 25 amino acids or 8 to 30 amino acids.

In another embodiment, the heterologous peptide or fragment thereof has a length of 3, 4, 5, 6, 7, 8, 9, 10, 11, 12, 13, 14, 15, 16, 17, 18, 19, 20, 21, 22, 23, 24, 25 amino acids.

1.2.3.2. Length CD4 Epitopes

In one embodiment, when the heterologous peptide or fragment thereof is a CD4 T cell epitope, said heterologous peptide or fragment thereof has a length of 5 to 25 amino acids, preferably 8 to 22 amino acids, more preferably 10 to 20 amino acids, even more preferably 12 to 18 amino acids.

In another embodiment, when the heterologous peptide or fragment thereof is a CD4 T cell epitope, said heterologous peptide or fragment thereof has a length of 5 to 10 amino acids, 5 to 15 amino acids, 5 to 18 amino acids, 5 to 20 amino acids or 5 to 25 amino acids.

In another embodiment, when the heterologous peptide or fragment thereof is a CD4 T cell epitope, said heterologous peptide or fragment thereof has a length of 6 to 10 amino acids, 6 to 15 amino acids, 6 to 18 amino acids, 6 to 20 amino acids or 6 to 25 amino acids.

In another embodiment, when the heterologous peptide or fragment thereof is a CD4 T cell epitope, said heterologous peptide or fragment thereof has a length of 7 to 10 amino acids, 7 to 15 amino acids, 7 to 18 amino acids, 7 to 20 amino acids or 7 to 25 amino acids.

In another embodiment, when the heterologous peptide or fragment thereof is a CD4 T cell epitope, said heterologous peptide or fragment thereof has a length of 8 to 10 amino acids, 8 to 15 amino acids, 8 to 18 amino acids, 8 to 20 amino acids or 8 to 25 amino acids.

In another embodiment, when the heterologous peptide or fragment thereof is a CD4 T cell epitope, said heterologous peptide or fragment thereof has a length of 9 to 10 amino acids, 9 to 15 amino acids, 9 to 18 amino acids, 9 to 20 amino acids or 9 to 25 amino acids.

In another embodiment, when the heterologous peptide or fragment thereof is a CD4 T cell epitope, said heterologous peptide or fragment thereof has a length of 10 to 15 amino acids, 10 to 18 amino acids, 10 to 20 amino acids or 10 to 25 amino acids.

In another embodiment, when the heterologous peptide or fragment thereof is a CD4 T cell epitope, said heterologous peptide or fragment thereof has a length of 11 to 15 amino acids, 11 to 18 amino acids, 11 to 20 amino acids or 11 to 25 amino acids.

In another embodiment, when the heterologous peptide or fragment thereof is a CD4 T cell epitope, said heterologous peptide or fragment thereof has a length of 12 to 15 amino acids, 12 to 18 amino acids, 12 to 20 amino acids or 12 to 25 amino acids.

In another embodiment, the heterologous peptide or fragment thereof has a length of 5, 6, 7, 8, 9, 10, 11, 12, 13, 14, 15, 16, 17, 18, 19, 20, 21, 22, 23, 24, 25 amino acids.

1.2.3.3. Length CD8 Epitopes

In one embodiment, when the heterologous peptide or fragment thereof is a CD8 T cell epitope, said heterologous peptide or fragment thereof has a length of 3 to 20 amino acids, preferably 3 to 15 amino acids, more preferably 5 to 13 amino acids, even more preferably 7 to 11 amino acids.

In one embodiment, when the heterologous peptide or fragment thereof is a CD8 T cell epitope, said heterologous peptide or fragment thereof has a length of 3 to 9 amino acids, 3 to 11 amino acids, 3 to 15 amino acids, 3 to 18 amino acids or 3 to 20 amino acids.

In one embodiment, when the heterologous peptide or fragment thereof is a CD8 T cell epitope, said heterologous peptide or fragment thereof has a length of 4 to 9 amino acids, 4 to 11 amino acids, 4 to 15 amino acids, 4 to 18 amino acids or 4 to 20 amino acids.

In one embodiment, when the heterologous peptide or fragment thereof is a CD8 T cell epitope, said heterologous peptide or fragment thereof has a length of 5 to 9 amino acids, 5 to 11 amino acids, 5 to 15 amino acids, 5 to 18 amino acids or 5 to 20 amino acids.

In one embodiment, when the heterologous peptide or fragment thereof is a CD8 T cell epitope, said heterologous peptide or fragment thereof has a length of 6 to 9 amino acids, 6 to 11 amino acids, 6 to 15 amino acids, 6 to 18 amino acids or 6 to 20 amino acids.

In one embodiment, when the heterologous peptide or fragment thereof is a CD8 T cell epitope, said heterologous peptide or fragment thereof has a length of 7 to 9 amino acids, 7 to 11 amino acids, 7 to 15 amino acids, 7 to 18 amino acids or 7 to 20 amino acids.

In another embodiment, when the heterologous peptide or fragment thereof is a CD8 T cell epitope, said heterologous peptide or fragment thereof has a length of 3, 4, 5, 6, 7, 8, 9, 10, 11, 12, 13, 14, 15, 16, 17, 18, 19, 20 amino acids.

1.2.3.4. Long CD4/CD8 Epitopes

In one embodiment, when the heterologous peptide or fragment thereof is a fragment of antigen comprising two T cell epitopes, said heterologous peptide or fragment thereof has a length of 20 to 100 amino acids, preferably 25 to 80 amino acids, more preferably 30 to 60 amino acids, even more preferably 30 to 45 amino acids, even more preferably 35 to 40 amino acids.

In one embodiment, when the heterologous peptide or fragment thereof is a fragment of antigen comprising two T cell epitopes, said heterologous peptide or fragment thereof has a length of 20 to 35 amino acids, 20 to 40 amino acids, 20 to 45 amino acids, 20 to 50 amino acids or 20 to 60 amino acids.

In one embodiment, when the heterologous peptide or fragment thereof is a fragment of antigen comprising two T cell epitopes, said heterologous peptide or fragment thereof has a length of 25 to 35 amino acids, 25 to 40 amino acids, 25 to 45 amino acids, 25 to 50 amino acids or 25 to 60 amino acids.

In one embodiment, when the heterologous peptide or fragment thereof is a fragment of antigen comprising two T cell epitopes, said heterologous peptide or fragment thereof has a length of 30 to 35 amino acids, 30 to 40 amino acids, 30 to 45 amino acids, 30 to 50 amino acids or 30 to 60 amino acids.

In one embodiment, when the heterologous peptide or fragment thereof is a fragment of antigen comprising two T cell epitopes, said heterologous peptide or fragment thereof has a length of 31 to 35 amino acids, 31 to 40 amino acids, 31 to 45 amino acids, 31 to 50 amino acids or 31 to 60 amino acids.

In one embodiment, when the heterologous peptide or fragment thereof is a fragment of antigen comprising two T cell epitopes, said heterologous peptide or fragment thereof has a length of 32 to 35 amino acids, 32 to 40 amino acids, 32 to 45 amino acids, 32 to 50 amino acids or 32 to 60 amino acids.

In one embodiment, when the heterologous peptide or fragment thereof is a fragment of antigen comprising two T cell epitopes, said heterologous peptide or fragment thereof has a length of 33 to 35 amino acids, 33 to 40 amino acids, 33 to 45 amino acids, 33 to 50 amino acids or 33 to 60 amino acids.

In one embodiment, when the heterologous peptide or fragment thereof is a fragment of antigen comprising two T cell epitopes, said heterologous peptide or fragment thereof has a length of 34 to 35 amino acids, 34 to 40 amino acids, 34 to 45 amino acids, 34 to 50 amino acids or 34 to 60 amino acids.

In one embodiment, when the heterologous peptide or fragment thereof is a fragment of antigen comprising two T cell epitopes, said heterologous peptide or fragment thereof has a length of 35 to 40 amino acids, 35 to 45 amino acids, 35 to 50 amino acids or 35 to 60 amino acids.

In another embodiment, when the heterologous peptide or fragment thereof is a fragment of antigen comprising two T cell epitopes, said heterologous peptide or fragment thereof has a length of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 amino acids.

In one embodiment, when the heterologous peptide or fragment thereof is a fragment of antigen comprising two or more T cell epitopes, said two or more T cell epitopes are separated by a small amino acid sequence, herein referred as to “spacer”.

In one embodiment, a spacer comprises between 0 and 50 amino acids, preferably between 2 and 25 amino acids, more preferably between 5 and 20 amino acids, more preferably between 7 and 15 amino acids.

In one embodiment, a spacer comprises 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids.

1.3. Insertion Method

In one embodiment, peptides of the invention are inserted into VSV-G by recombinant DNA methods. Nucleic acids of the present invention can be readily prepared by the skilled person using techniques known in the art (for example, see Sambrook et al., Molecular Cloning: A Laboratory Manual. New-York: Cold Spring Harbor Laboratory Press, 1989; Ausubel et al., Short Protocols in Molecular Biology. New-York: John Wiley and Sons, 1992). For example, the modified sequence of VSV-G is obtained by artificial gene synthesis. This allows an adaptation of codon usage for a better expression of the sequence (Angov et al., 2011. Biotechnol. J. 6(6):650-659). The optimized sequence is then subcloned into an expression vector. In another example, a synthetic nucleic acid sequence or vector containing a nucleic acid sequence encoding a peptide to be inserted into VSV-G is specifically designed to include restriction endonuclease sites matched to a specified endonuclease-cut nucleic acid sequence encoding VSV-G or to a specified endonuclease-cut nucleic acid sequence previously added into the VSV-G sequence. Where a desirable VSV-G insertion site contains a single, unique restriction endonuclease site, the peptide's nucleic acid sequence is preferably engineered to include matched restriction sites at both ends of the sequence. In this manner, the sequence encoding the peptide is inserted into the VSV-G sequence without removal of any VSV-G-encoding nucleotides. Care is taken to match the peptide-encoding nucleic acid sequence to be inserted with the reading frame of the VSV-G sequence so that normal expression of the encoded VSV-G with the encoded peptide of interest is achieved. Modified VSV-G can also result from Gibson assembly cloning where multiple DNA fragments can be assembled, regardless of fragment length or end compatibility.

In one embodiment, at least one heterologous peptide or antigen fragment is inserted into VSV-G at any VSV-G permissive insertion site, preferably at a VSV-G permissive epitope insertion site.

In one embodiment, the at least one heterologous peptide or fragment thereof is inserted into VSV-G, in highly variable regions. In one embodiment, said highly variable regions are defined on the basis of sequence alignments of VSV-G from various strains. These highly variable regions can undergo sequence modifications without affecting the stability and/or function of the protein. In one embodiment, said highly variable regions are regions which are exposed at the surface of the protein. In one embodiment, said highly variable regions are regions comprised in exposed turns, including α-turns, β-turns, γ-turns, δ-turns, π-turns, ω-turns, loops and/or hairpins. Suitable regions for inserting the at least one heterologous peptide or fragment thereof can be determined by methods known from the skilled person, using for example protein structure prediction software and/or loop modeling software.

In one embodiment, the at least one heterologous peptide or fragment thereof is inserted into VSV-G, at its C-terminal extremity, i.e., after the last amino acid residue of its sequence.

In one embodiment, the at least one heterologous peptide or fragment thereof is inserted into VSV-G from vesicular stomatitis Indiana virus (VSIV) (SEQ ID NO: 1) within region(s) selected from the group consisting of:
Region 1: amino acid residues 1 to 19 of SEQ ID NO: 1;
Region 2: amino acid residues 42 to 61 of SEQ ID NO: 1;
Region 3: amino acid residues 184 to 233 of SEQ ID NO: 1;
Region 4: amino acid residues 253 to 268 of SEQ ID NO: 1;
Region 5: amino acid residues 270 to 289 of SEQ ID NO: 1;
Region 6: amino acid residues 362 to 372 of SEQ ID NO: 1; and
Region 7: after amino acid residue 511, i.e., at the C-terminal extremity of SEQ ID NO: 1.

In one embodiment, the at least one heterologous peptide or fragment thereof is inserted into VSV-G from vesicular stomatitis New Jersey virus (VSNJV) (SEQ ID NO: 2) within region(s) selected from the group consisting of:
Region 1: amino acid residues 1 to 19 of SEQ ID NO: 2;
Region 2: amino acid residues 42 to 61 of SEQ ID NO: 2;
Region 3: amino acid residues 184 to 233 of SEQ ID NO: 2;
Region 4: amino acid residues 253 to 272 of SEQ ID NO: 2;
Region 5: amino acid residues 274 to 293 of SEQ ID NO: 2;
Region 6: amino acid residues 366 to 376 of SEQ ID NO: 2; and
Region 7: after amino acid residue 517, i.e., at the C-terminal extremity of SEQ ID NO:2.

In one embodiment, the at least one heterologous peptide or fragment thereof is inserted into VSV-G from Chandipura virus (CHPV) (SEQ ID NO: 3) within region(s) selected from the group consisting of:
Region 1: amino acid residues 1 to 24 of SEQ ID NO: 3;
Region 2: amino acid residues 47 to 66 of SEQ ID NO: 3;
Region 3: amino acid residues 189 to 237 of SEQ ID NO: 3;
Region 4: amino acid residues 257 to 276 of SEQ ID NO: 3;
Region 5: amino acid residues 278 to 297 of SEQ ID NO: 3;
Region 6: amino acid residues 370 to 381 of SEQ ID NO: 3; and
Region 7: after amino acid residue 530, i.e., at the C-terminal extremity of SEQ ID NO:3.

In one embodiment, the at least one heterologous peptide or fragment thereof is inserted into VSV-G from Cocal virus (COCV) (SEQ ID NO: 4) within region(s) selected from the group consisting of:
Region 1: amino acid residues 1 to 20 of SEQ ID NO: 4;
Region 2: amino acid residues 43 to 62 of SEQ ID NO: 4;
Region 3: amino acid residues 185 to 234 of SEQ ID NO: 4;
Region 4: amino acid residues 254 to 269 of SEQ ID NO: 4;
Region 5: amino acid residues 271 to 290 of SEQ ID NO: 4;
Region 6: amino acid residues 363 to 373 of SEQ ID NO: 4; and
Region 7: after amino acid residue 512, i.e., at the C-terminal extremity of SEQ ID NO:4.

In one embodiment, the at least one heterologous peptide or fragment thereof is inserted into VSV-G from Piry virus (PIRYV) (SEQ ID NO: 5) within region(s) selected from the group consisting of:
Region 1: amino acid residues 1 to 21 of SEQ ID NO: 5;
Region 2: amino acid residues 44 to 63 of SEQ ID NO: 5;
Region 3: amino acid residues 186 to 233 of SEQ ID NO: 5;
Region 4: amino acid residues 253 to 272 of SEQ ID NO: 5;
Region 5: amino acid residues 274 to 293 of SEQ ID NO: 5;
Region 6: amino acid residues 366 to 377 of SEQ ID NO: 5; and
Region 7: after amino acid residue 529, i.e., at the C-terminal extremity of SEQ ID NO:5.

In one embodiment, the at least one heterologous peptide or fragment thereof is inserted into VSV-G from Isfahan virus (ISFV) (SEQ ID NO: 6) within region(s) selected from the group consisting of:
Region 1: amino acid residues 1 to 23 of SEQ ID NO: 6;
Region 2: amino acid residues 46 to 65 of SEQ ID NO: 6;
Region 3: amino acid residues 188 to 236 of SEQ ID NO: 6;
Region 4: amino acid residues 256 to 275 of SEQ ID NO: 6;
Region 5: amino acid residues 277 to 296 of SEQ ID NO: 6;
Region 6: amino acid residues 369 to 380 of SEQ ID NO: 6; and
Region 7: after amino acid residue 523, i.e., at the C-terminal extremity of SEQ ID NO:6.

In one embodiment, the at least one heterologous peptide or fragment thereof is inserted into VSV-G from Spring viraemia of carp virus (SVCV) (SEQ ID NO: 7) within region(s) selected from the group consisting of:
Region 1: amino acid residues 1 to 20 of SEQ ID NO: 7;
Region 2: amino acid residues 44 to 63 of SEQ ID NO: 7;
Region 3: amino acid residues 186 to 235 of SEQ ID NO: 7;
Region 4: amino acid residues 254 to 270 of SEQ ID NO: 7;
Region 5: amino acid residues 272 to 291 of SEQ ID NO: 7;
Region 6: amino acid residues 364 to 374 of SEQ ID NO: 7; and
Region 7: after amino acid residue 509, i.e., at the C-terminal extremity of SEQ ID NO:7.

In one embodiment, the at least one heterologous peptide or fragment thereof is inserted into VSV-G from Alagoas virus (VSAV) (SEQ ID NO: 54) within region(s) selected from the group consisting of:
Region 1: amino acid residues 1 to 20 of SEQ ID NO: 54;
Region 2: amino acid residues 43 to 62 of SEQ ID NO: 54;
Region 3: amino acid residues 185 to 234 of SEQ ID NO: 54;
Region 4: amino acid residues 254 to 269 of SEQ ID NO: 54;
Region 5: amino acid residues 271 to 290 of SEQ ID NO: 54;
Region 6: amino acid residues 363 to 373 of SEQ ID NO: 54; and
Region 7: after amino acid residue 511, i.e., at the C-terminal extremity of SEQ ID NO:54.

In one embodiment, the at least one heterologous peptide or fragment thereof is inserted into VSV-G from Carajás virus (CJSV) (SEQ ID NO: 55) within region(s) selected from the group consisting of:
Region 1: amino acid residues 1 to 24 of SEQ ID NO: 55;
Region 2: amino acid residues 47 to 66 of SEQ ID NO: 55;
Region 3: amino acid residues 189 to 238 of SEQ ID NO: 55;
Region 4: amino acid residues 258 to 277 of SEQ ID NO: 55;
Region 5: amino acid residues 279 to 298 of SEQ ID NO: 55;
Region 6: amino acid residues 371 to 381 of SEQ ID NO: 55; and
Region 7: after amino acid residue 523, i.e., at the C-terminal extremity of SEQ ID NO:55.

In one embodiment, the at least one heterologous peptide or fragment thereof is inserted into VSV-G from Maraba virus (MARAV) (SEQ ID NO: 56) within region(s) selected from the group consisting of:
Region 1: amino acid residues 1 to 19 of SEQ ID NO: 56;
Region 2: amino acid residues 42 to 61 of SEQ ID NO: 56;
Region 3: amino acid residues 184 to 233 of SEQ ID NO: 56;
Region 4: amino acid residues 253 to 268 of SEQ ID NO: 56;
Region 5: amino acid residues 270 to 289 of SEQ ID NO: 56;
Region 6: amino acid residues 362 to 372 of SEQ ID NO: 56; and
Region 7: after amino acid residue 512, i.e., at the C-terminal extremity of SEQ ID NO:56.

In another embodiment, the at least one heterologous peptide or fragment thereof is inserted into VSV-G from a virus strain classified or provisionally classified in the Vesiculovirus genus such as Chandipura virus (CHPV), Cocal virus (COCV), Indiana virus (VSIV), Isfahan virus (ISFV), New Jersey virus (VSNJV), Piry virus (PIRYV), Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURV), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viraemia of carp virus (SVCV), Tupaia virus (TUPV), Ulcerative disease rhabdovirus (UDRV) and Yug Bogdanovac virus (YBV). The at least one heterologous peptide or fragment thereof is inserted in positions that are readily selected by the one skilled in the art.

As used hereafter, and unless indicated otherwise, the positions into which the heterologous peptide(s) is/are inserted are defined by the amino acid residue directly after the insertion site. In other words, insertion position 18 corresponds to the region between amino acid residues 17 and 18.

In one embodiment, the at least one heterologous peptide or fragment thereof is inserted into VSV-G from vesicular stomatitis Indiana virus (VSIV) (SEQ ID NO: 1) at a VSV-G amino acid position selected from the group comprising or consisting of positions 13, 14, 15, 16, 17, 18, 19, 20, 21, 22, 23, 46, 47, 48, 49, 50, 51, 52, 53, 54, 55, 56, 57, 58, 59, 60, 186, 187, 188, 189, 190, 191, 192, 193, 194, 195, 196, 197, 198, 199, 200, 201, 202, 212, 213, 214, 215, 216, 217, 218, 219, 220, 221, 222, 363, 364, 365, 366, 367, 368, 369, 370, 371, 372, 373 and C-terminal extremity, and combinations thereof.

In one embodiment, the at least one heterologous peptide or fragment thereof is inserted into VSV-G from vesicular stomatitis Indiana virus (VSIV) (SEQ ID NO: 1) at a VSV-G amino acid position selected from the group comprising or consisting of positions 18, 51, 55, 191, 196, 217, 368 and C-terminal extremity, and combinations thereof.

In a preferred embodiment, the at least one heterologous peptide or fragment thereof is inserted into VSV-G at VSV-G amino acid positions 18 and/or 191 with respect to SEQ ID NO: 1. In other words, in a preferred embodiment, the nucleic acid sequence encoding the heterologous peptide is inserted into the nucleic acid sequence encoding VSV-G such that the expressed modified VSV-G will include the heterologous peptide inserted at VSV-G amino acid position 18 and/or 191 with respect to SEQ ID NO: 1.

In another preferred embodiment, the at least one heterologous peptide or fragment thereof is inserted into VSV-G at the C-terminal extremity of VSV-G.

In a particular embodiment, more than one heterologous peptide or fragment thereof is inserted into VSV-G at VSV-G amino acid positions 18 with respect to SEQ ID NO: 1. In a particular embodiment, more than one heterologous peptide or fragment thereof is inserted into VSV-G at VSV-G amino acid positions 51 with respect to SEQ ID NO: 1. In a particular embodiment, more than one heterologous peptide or fragment thereof is inserted into VSV-G at VSV-G amino acid positions 55 with respect to SEQ ID NO: 1. In a particular embodiment, more than one heterologous peptide or fragment thereof is inserted into VSV-G at VSV-G amino acid positions 191 with respect to SEQ ID NO: 1. In a particular embodiment, more than one heterologous peptide or fragment thereof is inserted into VSV-G at VSV-G amino acid positions 196 with respect to SEQ ID NO: 1. In a particular embodiment, more than one heterologous peptide or fragment thereof is inserted into VSV-G at VSV-G amino acid positions 217 with respect to SEQ ID NO: 1. In a particular embodiment, more than one heterologous peptide or fragment thereof is inserted into VSV-G at VSV-G amino acid positions 368 with respect to SEQ ID NO: 1. In a particular embodiment, more than one heterologous peptide or fragment thereof is inserted into VSV-G at VSV-G C-terminal extremity.

Techniques to determine amino acid positions in a VSV-G other than VSV-G from vesicular stomatitis Indiana virus (VSIV) (SEQ ID NO: 1) into which at least one heterologous peptide or fragment thereof can be inserted are well-known in the art.

In one embodiment, multiple heterologous peptides may be inserted into VSV-G, e.g., at more than one site in VSV-G, preferably at two or more sites. In one embodiment, the modified VSV-G of the invention comprises multiple copies of the same heterologous peptide. In another embodiment, the modified VSV-G of the invention comprises one copy of different heterologous peptides. In still another embodiment, the modified VSV-G of the invention comprises one or more copies of different heterologous peptides.

2. Polynucleotide

A second aspect of the invention relates to a polynucleotide, or a nucleic acid sequence, coding for a modified VSV-G according to the invention.

A “coding sequence” or a sequence “encoding” a modified VSV-G, is a nucleotide sequence that, when expressed, results in the production of that modified VSV-G, i.e., the nucleotide sequence encodes an amino acid sequence for that modified VSV-G. In one embodiment, the coding sequence includes a start codon (usually ATG) and a stop codon.

In one embodiment, the polynucleotide or nucleic acid sequence is an isolated polynucleotide or an isolated nucleic acid sequence.

In one embodiment, polynucleotides or nucleic acids of the invention may be obtained by conventional methods well known to those skilled in the art. Typically, said polynucleotide or nucleic acid is a DNA or RNA molecule, which may be included in a suitable vector, such as a plasmid, cosmid, episome, artificial chromosome, phage or viral vector.

In one embodiment, the polynucleotide or nucleic acid of the invention is a DNA molecule. In another embodiment, the polynucleotide or nucleic acid of the invention is a RNA molecule.

In a particular embodiment, the polynucleotide or nucleic acid of the invention is a mRNA molecule.

In one embodiment, the codon usage bias of the polynucleotide or nucleic acid of the invention is optimized. As used herein, the term “codon usage bias” refers to the high-frequency preferential use of a particular codon (as opposed to other, synonymous codons) coding for an amino acid within a given organism, tissue or cell. A codon usage bias may be expressed as a quantitative measurement of the rate at which a particular codon is used in the genome of a particular organism, tissue or cell, for example, when compared to other codons that encode the same amino acid. Various methods are known to those of skill in the art for determining codon usage bias. In some embodiments, codon usage bias may be determined by the codon adaptation index (CAI) method, which is essentially a measurement of the distance of a gene's codon usage to the codon usage of a predefined set of highly-expressed genes (Sharp and Li, 1987. Nucleic Acids Res. 15:1281-95). Alternative methods for determining a codon usage bias include MILC (Measure Independent of Length and Composition) (Supek and Vlahovicek, 2005. BMC Bioinformatics. 6:182) and relative synonymous codon usage (RSCU), which is the observed frequency of a particular codon divided by the frequency expected from equal usage of all the synonymous codons for that amino acid (Sharp et al., 1986. Nucleic Acids Res. 14:5125-43). RSCU values close to 1.0 indicate a lack of bias for the particular codon, whereas departure from 1.0 reflects codon usage bias.

In one embodiment, one or more polynucleotides are inserted ex vivo into dendritic cells, such that one or more selected heterologous peptides, preferably antigens, are presented in effective amounts on the surface of the dendritic cells. By “effective amount” is meant that presentation is sufficient to enable the dendritic cells to provoke an immune response.

Techniques for nucleic acid manipulation are well known. Reagents useful in applying such techniques, such as restriction enzymes and the like, are widely known in the art and commercially available from a number of vendors.

Polynucleotides encoding the desired heterologous peptides, preferably antigens, for presentation in the dendritic cells are preferably recombinant expression vectors in which high levels of expression may occur.

In one embodiment, the vectors may also contain polynucleotide sequences encoding selected class I and class II MHC molecules, costimulation and other immunoregulatory molecules, ABC transporter proteins, including the TAP1 and TAP2 proteins. In one embodiment, the vectors may also contain at least one positive marker that enables the selection of dendritic cells carrying the inserted nucleic acids.

Expression of the polynucleotide of interest after transfection into dendritic cells may be confirmed by immunoassays or biological assays. For example, expression of introduced polynucleotides into cells may be confirmed by detecting the binding to the cells of labeled antibodies specific for the antigens of interest using assays well known in the art such as FACS (Fluorescent Activated Cell Sorting) or ELISA (enzyme-linked immunoabsorbent assay) or by simply by staining (e.g., with β-gal) and determining cell counts.

T cell activation may be detected by various known methods, including measuring changes in the proliferation of T cells, killing of target cells, tetramer staining, and secretion of certain regulatory factors, such as lymphokines, expression of mRNA of certain immunoregulatory molecules, or a combination of these.

3. Vector

Therefore, a further object of the present invention relates to a vector or a plasmid in which a polynucleotide of the invention is associated with suitable elements for controlling transcription (in particular promoter, enhancer and, optionally, terminator) and, optionally translation.

The present invention also relates to the recombinant vectors into which a polynucleotide in accordance with the invention is inserted. These recombinant vectors may, for example, be cloning vectors, or expression vectors.

The terms “vector”, “cloning vector” and “expression vector” mean the vehicle by which the polynucleotide of the invention may be introduced into a host cell, so as to transform the host and promote expression (e.g., transcription and translation) of the polynucleotide.

Any expression vector for animal cell may be used, as long as a polynucleotide encoding a modified VSV-G of the invention can be inserted and expressed. Examples of suitable vectors include, but are not limited to, pVAX2, pAGE107, pAGE103, pHSG274, pKCR, pSG1 β d2-4 and the like.

Other examples of plasmids include replicating plasmids comprising an origin of replication, or integrative plasmids, such as for instance pUC, pcDNA, pBR, and the like.

In one embodiment, the vector is devoid of antibiotic resistance gene. For example, selection is based either on the complementation of auxotrophic strain, toxin-antitoxin systems, operator-repressor titration, RNA markers, or on the overexpression of a growth essential gene. Minicircles or any other method that allow removing of the antibiotic resistance gene from the initial vector can also be used (Vandermeulen et al., 2011. Mol. Ther. 19(11):1942-49).

In one embodiment, the polynucleotide of the invention is ligated into an expression vector which has been specifically optimized for polynucleotide vaccinations. Elements include but are not limited to a transcriptional promoter, immunogenic epitopes, additional cistrons encoding immunoenhancing or immunomodulatory genes (such as ubiquitin), with their own promoters, transcriptional terminator, bacterial origin of replication, antibiotic resistance gene or another selection marker, and CpG sequences to stimulate innate immunity, all of which are well known to those skilled in the art. Optionally, the vector may comprise internal ribosome entry sites (IRES).

In one embodiment, the vector comprises tissue-specific promoters or enhancers to limit expression of the polynucleotide to a particular tissue type.

For example, the muscle creatine kinase (MCK) enhancer element may be desirable to limit expression of the polynucleotide to a particular tissue type. Myocytes are terminally differentiated cells which do not divide. Integration of foreign DNA into chromosomes appears to require both cell division and protein synthesis. Thus, limiting protein expression to non-dividing cells such as myocytes may be preferable.

A further example includes keratinocyte-specific promoters, melanocyte-specific promoters and dermal papilla-specific promoters, such as for instance: keratin (including keratin 5 (K5) and keratin 14 (K14) promoters for the basal layer of skin; keratin 1 (K1) and keratin 10 (K10) promoters for the suprabasal layer of skin), loricrin, involucrin, transglutaminase I, E-cadherin, elastin, filaggrin, α1 collagen, cornifin β, mCC10 or melanocortin 1 receptor (MCR1) promoters.

In one embodiment, tissue- or cell-specific promoters may be used to target the expression of the modified VSV-G to antigen-presenting cells.

Examples of other eukaryotic transcription promoters include, but are not limited to, the Rous sarcoma virus (RSV) promoter, the simian virus 40 (SV40) promoter, the human elongation factor-1 α (EF-1α) promoter and the human ubiquitin C (UbC) promoter.

Suitable vectors include any plasmid DNA construct encoding a polynucleotide of the invention, operatively linked to a eukaryotic promoter. Examples of such vectors include the pCMV series of expression vectors, commercially available from Stratagene (La Jolla, Calif.); or the pcDNA or pREP series of expression vectors by Invitrogen Corporation (Carlsbad, Calif.).

In another embodiment, the vector is a viral vector. In one embodiment, viral vectors include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, and the like. Detailed protocols for producing such replication-defective recombinant viruses may be found for instance in WO1995014785, WO1996022378, U.S. Pat. Nos. 5,882,877, 6,013,516, 4,861,719, 5,278,056 and WO1994019478.

4. Host Cell/Dendritic Cell

Another object of the invention is also a prokaryotic or eukaryotic host cell genetically transformed with at least one polynucleotide according to the invention.

The term “transformation” means the introduction of a “foreign” (i.e., extrinsic or extracellular) gene, DNA or RNA sequence (including plasmids and viral vectors), to a host cell, so that the host cell will express the introduced gene or sequence to produce a desired substance, typically a protein coded by the introduced gene or sequence. A host cell that receives and expresses introduced DNA or RNA has been “transformed”.

Preferably, for expressing and producing the proteins, and in particular the modified VSV-G according to the invention, eukaryotic cells, in particular mammalian cells, and more particularly human cells, will be chosen.

Typically, cell lines such as CHO, BHK-21, COS-7, C127, PER.C6 or HEK293 could be used, for their ability to process to the right post-translational modifications of the derivatives.

The construction of expression vectors in accordance with the invention, and the transformation of the host cells can be carried out using conventional molecular biology techniques. The modified VSV-G of the invention, can, for example, be obtained by culturing genetically transformed cells in accordance with the invention and recovering the derivative expressed by said cell, from the culture. They may then, if necessary, be purified by conventional procedures, known in themselves to those skilled in the art, for example by fractionated precipitation, in particular ammonium sulphate precipitation, electrophoresis, gel filtration, affinity chromatography, etc.

In particular, conventional methods for preparing and purifying recombinant proteins may be used for producing the modified VSV-G in accordance with the invention.

The present invention further relates to a dendritic cell transfected by polynucleotide(s) of the invention, i.e., a dendritic cell in which one or more polynucleotides according to the invention are inserted into.

Another object of the invention is a dendritic cell population transfected by a nucleic acid sequence or a vector according to the invention.

5. Composition

The present invention also relates to a composition comprising, consisting essentially of or consisting of a modified VSV-G, polynucleotide, vector or cell of the invention.

As used herein, the expression “consist essentially of” means that the composition to which it refers does not comprise any other active ingredient, i.e., an ingredient responsible for a physiologic or therapeutic response, other than the modified VSV-G, polynucleotide, vector or cell of the invention.

The present invention further relates to a pharmaceutical composition comprising, consisting essentially of or consisting of a modified VSV-G, polynucleotide, vector or cell of the invention and at least one pharmaceutically acceptable excipient. As used herein, the term “pharmaceutical composition” includes veterinary composition.

The present invention also relates to an immunogenic composition comprising, consisting essentially of or consisting of a modified VSV-G, polynucleotide, vector or cell of the invention.

6. Vaccine

The present invention also relates to a vaccine comprising the nucleic acid sequence coding for a modified VSV-G according to the invention, the vector comprising the nucleic acid sequence coding for a modified VSV-G according to the invention, the host cell genetically transformed with the nucleic acid sequence coding for a modified VSV-G according to the invention or the modified VSV-G according to the invention.

In one embodiment, the vaccine of the invention is a prophylactic vaccine.

By “prophylactic vaccine” is meant that the vaccine is to be administered before definitive clinical signs, diagnosis or identification of the disease. According to this embodiment, the vaccine is to be administered to prevent the disease.

If the vaccine appears to induce an effective, but short-lived immune response, prophylactic vaccines may also be designed to be used as booster vaccines. Such booster vaccines are given to individuals who have previously received a vaccination, with the intention of prolonging the period of protection.

In another embodiment, the vaccine is a therapeutic vaccine, i.e., is to be administered after first clinical signs, diagnosis or identification of the disease. According to this embodiment, the vaccine is to be administered to treat the disease.

6.1. Polynucleotide Vaccine

In one embodiment, the vaccine is a polynucleotide vaccine.

Immunization with polynucleotide is also referred to as “genetic immunization”, “RNA immunization” or “DNA immunization”.

Accordingly, in one embodiment, the vaccine of the invention comprises a polynucleotide encoding, or a nucleic acid sequence coding for, a modified VSV-G according to the invention.

In one embodiment, the vaccine of the invention is a DNA-based vaccine. Accordingly, in one embodiment, the vaccine of the invention comprises a DNA molecule encoding a modified VSV-G according to the invention.

In another embodiment, the vaccine of the invention is a RNA-based vaccine. Accordingly, in one embodiment, the vaccine of the invention comprises a RNA molecule, preferably a mRNA molecule, encoding a modified VSV-G according to the invention.

In one embodiment, the vaccine of the invention expresses more than one modified VSV-G.

Accordingly, in one embodiment, the vaccine of the invention expresses two modified VSV-G or more. In a particular embodiment, the vaccine of the invention expresses two modified VSV-G or more, wherein said modified VSV-G are different.

According to this embodiment, the polynucleotide vaccine of the invention may comprise two polynucleotides encoding, or two nucleic acid sequences coding for, two different modified VSV-G. Still according to this embodiment, the protein vaccine of the invention may comprise two different modified VSV-G.

In a preferred embodiment, the vaccine of the invention expresses a first modified VSV-G and a second modified VSV-G wherein the first modified VSV-G comprises a CD8 T cell epitope and wherein the second modified VSV-G comprises a CD4 T cell epitope.

The present invention further relates to a combination of:
(a) a first modified VSV-G, polynucleotide, vector, composition, cell or vaccine comprising a first heterologous peptide or a first combination of more than one heterologous peptide or nucleic acid sequence thereof; and
(b) a second modified VSV-G, polynucleotide, vector, composition, cell or vaccine comprising a second heterologous peptide or a second combination of more than one heterologous peptide or nucleic acid sequence thereof; wherein said first heterologous peptide or combination of more than one heterologous peptide or nucleic acid sequence thereof and said second heterologous peptide or combination of more than one heterologous peptide or nucleic acid sequence thereof are different.

In one embodiment, said first heterologous peptide or nucleic acid sequence thereof is a CD8 T cell epitope and said second heterologous peptide or nucleic acid sequence thereof is a CD4 T cell epitope.

In one embodiment, said first and/or second modified VSV-G, polynucleotide, vector, composition, cell or vaccine may further comprise a universal antigenic CD4 T cell epitope or nucleic acid sequence thereof.

6.2. Protein Vaccine

In another embodiment, the vaccine of the invention is a protein vaccine. Accordingly, in one embodiment, the vaccine of the invention comprises a modified VSV-G according to the invention. In another embodiment, the vaccine of the invention comprises two modified VSV-G or more. In a particular embodiment, the vaccine of the invention comprises two modified VSV-G or more, wherein said modified VSV-G are different.

In a preferred embodiment, the vaccine of the invention comprises a first modified VSV-G and a second modified VSV-G wherein the first modified VSV-G comprises a CD8 T cell epitope and wherein the second modified VSV G comprises a CD4 T cell epitope.

In one embodiment, the vaccine of the present invention is used in a prime-boost strategy to induce robust and long-lasting immune response to the peptide. Priming and boosting vaccination protocols based on repeated injections of the same antigenic construct are well known and result in strong CTL responses. In general, the first dose may not produce protective immunity, but only “primes” the immune system. A protective immune response develops after the second or third dose.

In one embodiment, the vaccine of the invention is used in a conventional prime-boost strategy, in which the same vaccine is to be administered to the subject in multiple doses. In a preferred embodiment, the vaccine is used in one or more inoculations. These boosts are performed according to conventional techniques, and can be further optimized empirically in terms of schedule of administration, route of administration, choice of adjuvant, dose, and potential sequence when administered with another vaccine, therapy or homologous vaccine.

In another embodiment, the vaccine of the present invention is used in a prime-boost strategy using an alternative administration of modified VSV-G comprising xenoantigen and autoantigen or fragment thereof, or of polynucleotides encoding modified VSV-G comprising xenoantigen and autoantigen or fragment thereof. Specifically, according to the present invention, the subject is first treated, or “primed”, with a vaccine encoding an antigen of foreign origin or fragment thereof (a “xenoantigen”). Subsequently, the subject is then treated with another vaccine encoding an antigen or fragment thereof which is corresponding to the xenoantigen, but is of self-origin (“autoantigen”). This way, the immune reaction to the antigen is boosted. The boosting step may be repeated one or more times.

6.3. Excipients

In one embodiment, vaccines of the present invention are formulated with pharmaceutically acceptable carriers or excipients such as water, saline, dextrose, glycerol, and the like, as well as combinations thereof. In one embodiment, vaccines may also contain auxiliary substances such as wetting agents, emulsifying agents, buffers, adjuvants, and the like.

In another embodiment, excipient for use in the polynucleotide vaccines of the present invention is a polymer such as a cationic polymer or a non-ionic polymer (including but not limited to: polyoxyethylene (POE), polyoxypropylene (POP), polyethyleneglycol (PEG), linear or branched polyethylenimine (PEI)). In another embodiment, polymers can form block copolymers, for instance, a POE-POP-POE block copolymer. As used herein, the term “polyplex” refers to polymer-polynucleotide or copolymer-polynucleotide complexes.

In another embodiment, the polynucleotide vaccines are formulated with cationic lipids. Optionally, lipids can be mannolysated. As used herein, the term “lipoplex” refers to lipid-polynucleotide or liposome-polynucleotide complexes.

In one embodiment, lipoplexes are further complexed with polymers or copolymers to form tertiary complexes. These tertiary complexes have enhanced in vivo delivery and transfection capacities of the polynucleotide to the targeted cells, and thereby, facilitate enhanced immune responses.

In one embodiment, carries for use in the polynucleotide vaccines of the present invention are nanoparticles. These include but are not limited to: nano-emulsions, dendrimers, nano-gold, lipid-based nanoparticles, liposomes, drug-carrier conjugates, antibody-drug complexes, and magnetic nanoparticles.

6.4. Adjuvants

In one embodiment, the polynucleotide vaccine of the present invention is formulated with at least one adjuvant which may increase immunogenicity of the polynucleotide vaccines of the present invention. It is within the purview of the skilled artisan to utilize available adjuvants which may increase the immune response of the polynucleotide vaccines of the present invention in comparison to administration of a non-adjuvanted polynucleotide vaccine.

In some embodiments, the adjuvant is selected from the group consisting of α-interferon, γ-interferon, platelet derived growth factor (PDGF), TNF-α, TNF-β, GM-CSF, epidermal growth factor (EGF), HIV-1 gag, cutaneous T cell-attracting chemokine (CTACK), epithelial thymus-expressed chemokine (TECK), mucosae-associated epithelial chemokine (MEC), IL-2, IL-12, IL-15, IL-28, MHC, CD80, CD86 including IL-15 having the signal sequence deleted and optionally including the signal peptide from IgE. Other genes which may be useful adjuvants include those encoding: MCP-I, MIP-loc, MIP-I p, IL-8, RANTES, L-selectin, P-selectin, E-selectin, CD34, GIyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LlGAND, NKG2D, MICA, MICB, NKG2A, NKG2B, NKG2C, NKG2E, NKG2F, TAP1, TAP2, functional fragments and combinations thereof.

In some preferred embodiments, the adjuvant is selected from the group consisting of α-interferon, γ-interferon, IL-2, IL-8, IL-12, IL-15, IL-18, IL-28, MCP-I, MIP-Ia, MIP-Ip, RANTES, RANK, RANK LIGAND, Ox40, Ox40 LIGAND, CTACK, TECK, MEC, functional fragments and combinations thereof.

In some preferred embodiments, the adjuvant is selected from the group consisting of α-interferon, γ-interferon, IL-2, IL-12, functional fragments and combinations thereof.

In another embodiment, adjuvant for use in the polynucleotide vaccines of the present invention is mineral-based compounds such as one or more forms of an aluminum phosphate-based adjuvant, or one or more forms of a calcium phosphate.

In another embodiment, adjuvant is saponin, monophosphoryl lipid A or other compounds that can be used to increase immunogenicity of the polynucleotide vaccine.

In one embodiment, the polynucleotide vaccine of the present invention is formulated with at least one genetic adjuvant which may increase immunogenicity of the polynucleotide vaccines of the present invention. It is within the purview of the skilled artisan to utilize available genetic adjuvants which may increase the immune response of the polynucleotide vaccines of the present invention in comparison to administration of a non-adjuvanted polynucleotide vaccine.

As used herein, genetic adjuvants refer to immunomodulatory molecules encoded by a plasmid vector. They stimulate the innate immune system to trigger appropriate dendritic cell maturation and thereby a robust, specific, and long-lasting adaptive immune response. Immunomodulatory molecules include cytokines, chemokines, or immune stimulatory molecules, such as toll-like receptor agonists or interferon regulatory factors.

In one embodiment, the genetic adjuvant is not encoded by the polynucleotide or vector coding for a modified VSV-G according to the invention. In another embodiment, the genetic adjuvant is encoded by the polynucleotide or vector coding for a modified VSV-G according to the invention. According to this embodiment, the genetic adjuvant can be under the control of its own promoter; or the genetic adjuvant can be under the control of the same promoter as the modified VSV-G according to the invention, separated therefrom by an Internal Ribosome Entry Site (IRES).

In some embodiments, the genetic adjuvant is selected from the group consisting of α-interferon, γ-interferon, platelet derived growth factor (PDGF), TNF-α, TNF-β, GM-CSF, epidermal growth factor (EGF), HIV-1 gag, cutaneous T cell-attracting chemokine (CTACK), epithelial thymus-expressed chemokine (TECK), mucosae-associated epithelial chemokine (MEC), IL-2, IL-12, IL-15, IL-28, MHC, CD80, CD86 including IL-15 having the signal sequence deleted and optionally including the signal peptide from IgE. Other genes which may be useful adjuvants include, without limitation, those encoding MCP-I, MIP-loc, MIP-I p, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2, functional fragments and combinations thereof.

In some preferred embodiments, the genetic adjuvant is selected from the group consisting of α-interferon, γ-interferon, IL-2, IL-8, IL-12, IL-15, IL-18, IL-28, MCP-I, MIP-Ia, MIP-Ip, RANTES, RANK, RANK LIGAND, Ox40, Ox40 LIGAND, CTACK, TECK, MEC, functional fragments and combinations thereof.

In some preferred embodiments, the genetic adjuvant is selected from the group consisting of α-interferon, γ-interferon, IL-2, IL-12, functional fragments and combinations thereof.

Examples of other adjuvants include, but are not limited to, particle bombardment using DNA-coated or RNA-coated gold beads; co-administration of polynucleotide vaccines with plasmid DNA expressing cytokines, chemokines, or costimulatory molecules.

7. Use

A further object of the present invention relates to a modified VSV-G, polynucleotide, vector, composition, cell or vaccine according to the invention for use in the prevention or treatment of, or for use in preventing or treating, a disease or condition.

In one embodiment, the modified VSV-G, polynucleotide, vector, composition, cell or vaccine according to the invention is for use in the prevention or treatment of, or for use in preventing or treating, a cancer or an infectious disease.

In a particular embodiment, the modified VSV-G, polynucleotide, vector, composition, cell or vaccine according to the invention is used to provide long term inhibition of tumor growth in a subject.

According to an embodiment of the invention, dendritic cells transfected by polynucleotides of the invention are used to activate T cells in vitro. T cells or a subset of T cells can be obtained from various lymphoid tissues. Examples of such tissues include, but are not limited to, spleens, lymph nodes and peripheral blood.

The cells can be co-cultured with transfected dendritic cells as a mixed T cell population or as a purified T cell subset. For instance, it may be desired to culture purified CD8+ T cells with antigen transfected dendritic cells, as early elimination of CD4+ T cells may prevent the overgrowth of CD4+ cells in a mixed culture of both CD8+ and CD4+ T cells. T cell purification may be achieved by positive or negative selection, including, but not limited to, the use of antibodies directed to CD2, CD3, CD4, CD5, and CD8. On the other hand, it may be desired to use a mixed population of CD4+ and CD8+ T cells to elicit a specific response encompassing both a cytotoxic and Th immune response.

In one embodiment, after activation in vitro, the T cells may be administered to a subject in a dose sufficient to induce or enhance an immune response to the selected antigen expressed by the dendritic cells of the invention.

8. Administration/Doses

In one embodiment, the composition or vaccine of the invention is to be administered ex vivo or in vivo.

Ex vivo administration refers to performing part of the regulatory step outside of the subject, such as administering a composition of the present invention to a population of cells, preferably dendritic cells, removed from a subject under conditions such that the modified VSV-G, polynucleotide or vaccine is loaded into the cell, and returning the cells to the subject.

In one embodiment, the composition or vaccine of the invention may be administered to a subject, or returned to a subject, by any suitable mode of administration.

In one embodiment, the administration is systemic, mucosal and/or proximal to the location of the target site (e.g., near a tumor).

The preferred routes of administration will be apparent to those of skill in the art, depending on the type of condition to be prevented or treated, the antigen used and/or the target cell population or tissue.

Preferred methods of administration include, but are not limited to, electroporation or sonoporation. Administration by electroporation involves the application of a pulsed electric field to create transient pores in the cellular membrane without causing permanent damage to the cell. Administration by sonoporation involves the application of pulsed ultrasonic frequencies to create transient pores in the cellular membrane without causing permanent damage to the cell. It thereby allows for the introduction of exogenous molecules. By adjusting the electrical pulse and/or the ultrasonic frequencies, nucleic acid molecules can travel through passageways or pores in the cell that are created during the procedure.

Other preferred methods of administration include, but are not limited to, intravenous administration, intraperitoneal administration, intramuscular administration, intranodal administration, intracoronary administration, intraarterial administration (e.g., into a carotid artery), subcutaneous administration, intradermal administration, transdermal delivery, intratumoral administration, peritumoral administration, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue. In some embodiments, administration may be a combination of two or more of the various routes of administration.

Particularly preferred routes of administration include, but are not limited to, electroporation, sonoporation, intravenous, intraperitoneal, subcutaneous, intratumoral, peritumoral, intradermal, intranodal, intramuscular, transdermal, inhaled, intranasal, oral, intraocular, intraarticular, intracranial and intraspinal.

Parenteral delivery includes, without limitation, electroporation, sonoporation, intratumoral, peritumoral, intradermal, intramuscular, intraperitoneal, intrapleural, intrapulmonary, intravenous, subcutaneous, atrial catheter and venal catheter routes.

Aural delivery includes, without limitation, ear drops, intranasal delivery can include nose drops or intranasal injection, and intraocular delivery can include eye drops.

Aerosol (inhalation) delivery can also be performed using methods standard in the art (see, for example, Stribling et al., 1992. Proc. Natl. Acad. Sci. USA. 189:11277-11281). For example, in one embodiment, a composition or vaccine of the invention can be formulated into a composition suitable for nebulized delivery using a suitable inhalation device or nebulizer.

Oral delivery includes, without limitation, solids and liquids that can be taken through the mouth, and is useful in the development of mucosal immunity and since compositions comprising yeast vehicles can be easily prepared for oral delivery, for example, as tablets or capsules, as well as being formulated into food and beverage products.

Other routes of administration that modulate mucosal immunity are useful in the treatment of viral infections, epithelial cancers, immunosuppressive disorders and other diseases affecting the epithelial region. Such routes include bronchial, intradermal, intramuscular, intranasal, other inhalatory, rectal, subcutaneous, topical, transdermal, vaginal and urethral routes.

In one embodiment, the composition or vaccine may be administered to the subject by intramuscular injection, intradermal injection, gene gun, electroporation or biojector. In a more preferred embodiment, the composition or vaccine is to be administered by electroporation, preferably by intramuscular or intradermal electroporation.

Electroporation uses pulsed electric currents to open pores in cell membranes (a process called permeabilization) and allows the injected polynucleotide to be taken up by cells and immune cells residing in the tissue.

In one embodiment, the polynucleotide is formulated as lipoplex (cationic liposome-DNA complex), polyplex (cationic polymer-DNA complex), or protein-DNA complex.

In one embodiment, the composition or vaccine of the present invention is to be administered before symptoms appear, i.e., the composition or vaccine of the present invention is to be administered prophylactically.

In one embodiment, the composition or vaccine of the present invention is to be administered after symptoms appear, i.e., the composition or vaccine of the present invention is to be administered therapeutically.

According to the present invention, an effective administration protocol (i.e., administering a composition or vaccine in an effective manner) comprises suitable dose parameters and modes of administration that result in elicitation of an immune response in a subject that has a disease or condition, or that is at risk of contracting a disease or condition, preferably so that the subject is protected from the disease.

Effective dose parameters can be determined using methods standard in the art for a particular disease. Such methods include, but are not limited to, determination of survival rates, side effects (i.e., toxicity) and progression or regression of disease.

In particular, the effectiveness of dose parameters of a therapeutic composition of the present invention when treating cancer can be determined by assessing response rates. Such response rates refer to the percentage of treated patients in a population of patients that respond with either partial or complete remission. Remission can be determined by, for example, measuring tumor size or microscopic examination for the presence of cancer cells in a tissue sample.

According to the present invention, a suitable single dose size is a dose that is capable of eliciting an antigen-specific immune response in a subject when administered once or more times over a suitable time period. Doses can vary depending upon the disease or condition being treated. In the treatment of cancer, for example, a therapeutic effective amount can be dependent upon whether the cancer being treated is a primary tumor or a metastatic form of cancer. One of skills in the art can readily determine prophylactic or therapeutic effective amounts for administration based on the size of a subject and the route of administration.

In one embodiment, a prophylactic or therapeutic effective amount of the composition or vaccine of the invention is from about 0.5 pg to about 5 mg per kilogram body weight of the subject being administered the composition or vaccine. In a preferred embodiment, a prophylactic or therapeutic effective amount of the composition or vaccine of the invention is from about 0.1 μg to about 1 mg per kilogram body weight of the subject, preferably from about 1 μg to about 100 μg per kilogram body weight of the subject, preferably from about 10 μg to about 75 μg per kilogram body weight of the subject, preferably about 50 μg per kilogram body weight of the subject.

When T cells or dendritic cells are administered to a subject, the cells may be administered (with or without adjuvant) parenterally (including, for example, intravenous, intraperitoneal, intramuscular, intradermal, and subcutaneous administration). Alternatively, the cells may be administered locally by direct injection into a tumor or infected tissue.

Adjuvants include any known pharmaceutically acceptable carrier. Parenteral vehicles for use as pharmaceutical carriers include, but are not limited to, sodium chloride solution, Ringer's dextrose, dextrose and sodium chloride, and lactated Ringer's. Other adjuvants may be added as desired such as antimicrobials.

As an example, T cells may be administered by intravenous infusion, at doses of about 10⁸ to 10⁹ cells/m² of body surface area (see, e.g., Ridell et al., 1992. Science. 257:238-241). Infusion can be repeated at desired intervals, for example, monthly. Recipients are monitored during and after T cell infusions for any evidence of adverse effects.

According to a preferred embodiment, the T cells are obtained from the same subject from whom the dendritic cells were obtained.

According to another embodiment, the T cells are obtained from a subject and the dendritic cells, which are used to stimulate the T cells, are obtained from an HLA-matched healthy donor (e.g., a sibling), or vice versa.

According to yet another embodiment, both the T cells and the dendritic cells are obtained from an HLA-matched healthy donor. This embodiment may be particularly advantageous, for example, when the subject is a late stage cancer patient who has been treated with radiation and/or chemotherapy agents and may not be able to provide sufficient or efficient dendritic or T cells.

According to another embodiment of the invention, dendritic cells isolated from a subject are cultured, transfected in vitro and administered back to the subject to stimulate an immune response, including T cell activation. As such, the dendritic cells constitute a vaccine and/or immunotherapeutic agent.

As an example, dendritic cells presenting antigen are administered, via intravenous infusion, at a dose of, for example, about 10 to 10⁸ cells. According to an embodiment, dendritic cells presenting antigen are administered at a dose from about 0.5×10⁶ to about 40×10⁷ dendritic cells per administration, preferably from about 1×10⁶ to about 20×10⁷ dendritic cells per administration, more preferably from about 10×10⁶ to about 1×10⁷ dendritic cells per administration.

In one embodiment, infusion can be repeated at desired intervals based upon the subject's immune response.

When vaccines of the invention are used in a prime-boost strategy, “boosters” of the vaccine are preferably administered when the immune response against the peptide, preferably antigen, has waned or as needed to provide an immune response or induce a memory response against a particular peptide, preferably antigen. Boosters can be administered from about 1 week to several years after the original administration. In one embodiment, an administration schedule is one in which from about 0.5 pg to about 5 mg of a vaccine per kilogram body weight of the subject is to be administered from about one to about 4 times over a time period of from about 1 month to about 6 months.

It will be obvious to one of skills in the art that the number of doses administered to a subject is dependent upon the extent of the disease and the response of said subject to the treatment.

For example, a large tumor may require more doses than a smaller tumor, and a chronic disease may require more doses than an acute disease. In some cases, however, a subject having a large tumor may require fewer doses than a patient with a smaller tumor, if the subject with the large tumor responds more favorably to the composition or vaccine than the subject with the smaller tumor. Thus, it is within the scope of the present invention that a suitable number of doses includes any number required to treat a given disease.

9. Diseases

9.1. Cancer

In one embodiment, the disease or condition which may be prevented or treated with the modified VSV-G, polynucleotide, vector, composition, cell or vaccine according to the invention is a cancer.

As used herein, the term “cancer” includes, but is not limited to, solid tumors and blood borne tumors. The term cancer includes diseases of the skin, tissues, organs, bone, cartilage, blood and vessels.

In one embodiment, the cancer is a primary cancer. In another embodiment, the cancer is a metastatic cancer. A metastatic cancer is a cancer that has spread from its primary origin to another part of the body, also referred to as “late stage cancer” or “advanced stage cancer”. In some embodiments, advanced stage cancer includes stages 3 and 4 cancers. Cancers are ranked into stages depending on the extent of their growth and spread through the body; stages correspond with severity. Determining the stage of a given cancer helps doctors to make treatment recommendations, to form a likely outcome scenario for what will happen to the patient (prognosis), and to communicate effectively with other doctors.

Examples of cancer include, but are not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, hepatic cancers, lung cancers, pancreatic cancers, gastrointestinal cancers, renal cell carcinomas, hematopoietic neoplasias and metastatic cancers thereof.

In a particular embodiment, cancer is selected from the group comprising or consisting of melanomas, prostatic cancers, ovarian cancers, brain cancers, lung cancers and others.

Preferably, expression of the tumor antigen in a tissue of a subject, i.e., an animal or a human, that has cancer produces a result selected from the group of alleviation of the cancer, reduction of a tumor associated with the cancer, elimination of a tumor associated with the cancer, prevention of metastatic cancer, prevention of the cancer and stimulation of effector cell immunity against the cancer.

9.2. Infectious Diseases

In one embodiment, the disease or condition which may be prevented or treated with the modified VSV-G, polynucleotide, vector, composition, cell or vaccine according to the invention is an infectious disease.

In one embodiment, the infectious disease is selected from the group consisting of viral, bacterial, fungal and parasitic infection.

Examples of infectious virus include, but are not limited to, Retroviridae (e.g., human immunodeficiency viruses, such as HIV-1, also referred to as HTLV-III, LAV or HTLV-III/LAV, or HIV-III; and other isolates, such as HIV-LP); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever virus); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herperviridae (herpes simplex virus (HSV) 1 and 2, varicella zoster virus, cytomegalovirus (CMV), herpes viruses); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitides (thought to be a defective satellite of hepatitis B virus), the agents of non-A, non-B hepatitis (class 1-internally transmitted; class 2-parenterally transmitted (i.e., Hepatitis C); Norwalk and related viruses, and astroviruses).

Examples of infectious bacteria include, but are not limited to, Helicobacter pyloris, Boreliai burgdorferi, Legionella pneumophilia, Mycobacteria sps (e.g., M. tuberculosis, M. avium, M. Intracellulare, M. kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter erogenes, Klebsiella pneuomiae, Pasteurella multicoda, Bacteroides sp., Fusobacterium nucleatum, Sreptobacillus moniliformis, Treponema pallidium, Treponema pertenue, Leptospira, and Actinomeyces israelli.

Examples of infectious fungi include, but are not limited to, Cryptococcus neoformans, Histoplasma capsulatum, Coccidioides immitis, Blastomyces dermatitidis, Chlamydia trachomatis, Candida albicans. Other infectious organisms (i.e., protists) include, but are not limited to, Plasmodium falciparum and Toxoplasma gondii.

10. Subject

In one embodiment, the subject is susceptible or suspected of having a disease or condition, preferably a cancer or an infectious disease.

In one embodiment, the subject is at risk of developing a disease or condition, preferably a cancer or an infectious disease.

Examples of risks of developing a cancer include, but are not limited to, age, alcohol, exposure to cancer-causing substances, chronic inflammation, diet, hormones, familial cancer predisposition, genetic cancer predisposition, immunosuppression, infectious agents, obesity, exposure to radiation, exposure to sunlight, tobacco and the like.

Examples of risks of developing an infectious disease include, but are not limited to, exposure to bacteria, viruses, fungi, and parasites (for instance by indirect contact, insect bites or food contamination); having certain types of cancer or HIV; taking of steroids; implanted medical devices; malnutrition; extremes of age and the like.

In another embodiment, the subject suffers from a disease or condition, preferably a cancer or an infectious disease.

In one embodiment, the subject was not treated previously with another treatment for the disease or condition.

In another embodiment, the subject previously received one, two or more other treatments for the disease or condition. In one embodiment, the subject previously received one or more other treatments for the disease or condition, but was unresponsive or did not respond adequately to these treatments, which means that there is no or too low therapeutic benefit induced by these treatments.

In one embodiment, the subject is an animal, preferably a mammal.

In a further embodiment, said mammal is a domestic animal. As used herein, the term “domestic animal” refers to any of various animals domesticated so as to live and breed in a tame (as opposed to wild) condition. Domestic animals include, but are not limited to, cattle (including cows), horses, pigs, sheep, goats, dogs, cats, and any other mammal which is awaiting the receipt of, or is receiving medical care or was/is/will be the object of a medical procedure, or is monitored for the development of a disease.

In another embodiment, said mammal is a primate. As used herein, the term “primate” includes non-human primates such as lemurs, galagos, lorisids, tarsiers, monkeys, apes; and human primates, i.e., human.

In one embodiment, the subject of the invention is young. As used herein, the term “young” means that the subject is at most 20 years old, at most 15 or 10 years old if the subject is a human; or has an equivalent age according to the specie if the subject is a non-human animal.

In one embodiment, the subject is a child. As used herein, the term “child” refers to a human being (person) during the period between birth and puberty. By “puberty” it means the time in which sexual and physical characteristics mature person because of hormonal changes. In a particular embodiment, the present invention child is considered a person of up to 14 years (inclusive).

In one embodiment, the subject is a male. In another embodiment, the subject is a female. In one embodiment, the subject is a man. In another embodiment, the subject is a woman.

11. Method

Another object of the present invention is a method for preventing and/or treating a disease or a condition comprising administering a modified VSV-G, polynucleotide, vector, composition, cell or vaccine of the invention to a subject in need thereof.

In a particular embodiment, the method of the invention is for preventing and/or treating a cancer in a subject in need thereof, comprising administering a modified VSV-G, polynucleotide, vector, composition, cell or vaccine of the invention to said subject.

In another particular embodiment, the method of the invention is for preventing and/or treating an infectious disease in a subject in need thereof, comprising administering a modified VSV-G, polynucleotide, vector, composition, cell or vaccine of the invention to said subject.

In one embodiment, the method comprises administering a modified VSV-G, polynucleotide, vector, composition, cell or vaccine of the invention before symptoms appear. According to this embodiment, the method may be a prophylactic method.

In another embodiment, the method comprises administering a modified VSV-G, polynucleotide, vector, composition, cell or vaccine of the invention after first symptoms appear. According to this embodiment, the method may be a therapeutic method.

In one embodiment, the method of the invention is combined with other prophylactic and/or therapeutic approaches to enhance the efficacy of the method. For example, in the treatment of cancer, the modified VSV-G, polynucleotide, vector, composition, cell or vaccine of the invention may be administered after surgical resection of a tumor from the subject.

In another embodiment, the modified VSV-G, polynucleotide, vector, composition, cell or vaccine of the invention may be administered in combination with another therapeutic molecule, such as chemotherapeutic agents, anti-angiogenesis agents, checkpoint blockade antibodies or other molecules that reduce immune-suppression; or in combination with another antitumor treatment, such as radiation therapy, hormonal therapy, targeted therapy or immunotherapy.

In a particular embodiment, the modified VSV-G, polynucleotide, vector, composition, cell or vaccine of the invention is to be administered in combination with antibodies. Examples of antibodies which may be co-administered include, but are not limited to, antibodies anti-PD-1 (e.g., nivolumab, pidilizumab and MK-3475), antibodies anti-PD-L1 (e.g., BMS-936559, MEDI4736 and MPDL33280A), antibodies anti-CTLA4 (e.g., ipilimumab and tremelimumab), antibodies anti-OX40, antibodies anti-4-1BB, antibodies anti-CD47, antibodies anti-KIR, antibodies anti-CD40, antibodies anti-LAG-3 and combinations thereof.

In a particular embodiment, the modified VSV-G, polynucleotide, vector, composition, cell or vaccine of the invention is to be administered in combination with stimulating factors. Example of stimulating factors which may be co-administered include, but are not limited to, granulocyte-macrophage colony-stimulating factor (GM-CSF) (e.g., sargramostim or molgramostim).

Another object of the present invention is a method for inducing in a subject a protective immune response comprising administering a modified VSV-G, polynucleotide, vector, composition, cell or vaccine of the invention to a subject in need thereof.

In one embodiment, the method of the invention is for inducing in a subject a protective immune response against cancer. In another embodiment, the method of the invention is for inducing in a subject a protective immune response against a pathogen.

11.1. Personalized Treatment

The present invention also relates to a personalized method for treating a disease or condition, preferably a cancer, in a subject (i.e., a human being or a non-human animal) in need thereof comprising administering a modified VSV-G, polynucleotide, vector, cell, composition or vaccine as described herein above.

In one embodiment, the personalized method for treating a cancer in a subject in need thereof comprises the steps of:
a) providing a sample of a tumor from a subject;
b) identifying at least one neoantigen;
c) preparing a composition comprising the at least one neoantigen inserted into VSV-G; and
d) administering the composition to the subject.

In one embodiment, the personalized method for treating a cancer in a subject in need thereof comprises the steps of:
a) providing a sample of a tumor from a subject;
b) identifying at least one neoantigen;
c) preparing a composition comprising a polynucleotide encoding a modified VSV-G into which a polynucleotide encoding the at least one neoantigen is inserted; and
d) administering the composition to the subject.

Any cell type or tissue may be utilized to obtain nucleic acid samples for use in the sequencing methods described herein. In a preferred embodiment, the DNA or RNA sample is obtained from a sample of a tumor from a subject or a bodily fluid, e.g., blood, obtained by known techniques (e.g., venipuncture), saliva, sweat, urine, feces, vomit, breast milk and semen. Alternatively, nucleic acid tests can be performed on dry samples (e.g., hair or skin).

Methods for identifying neoantigens are well-known from the person skilled in the art.

For example, tumor sample from a subject and normal tissue may be subjected to whole-exome sequencing and RNA-Seq to identify expressed nonsynonymous somatic mutations. These mutations may be pipelined into an epitope prediction algorithm (such as for example IEDB, EpiBot, EpiToolKit) to prioritize a list of candidate antigens and/or may be expressed as minigenes used for the identification and expansion of mutant neoantigen-specific autologous T cells isolated from blood or tumor of the same subject. Ex vivo-expanded T cells may be then infused back into the cancer patient.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLiD system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention.

A variety of methods are available for detecting the presence of a particular mutation or allele in an individual's DNA or RNA.

Examples of such methods include, but are not limited to, dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR.

Examples that eliminate the need for PCR include methods based on the generation of small signal molecules by invasive cleavage followed by mass spectrometry or immobilized padlock probes and rolling-circle amplification.

Alternatively, expressed mutations predicted to form neoantigens by MHC class I epitope-binding algorithms may be confirmed and then used to generate neoantigen vaccines.

Tumor-specific neoantigens may also be identified using MHC multimers to identify neoantigen-specific T cell responses. For example, high throughput analysis of neoantigen-specific T cell responses in patient samples may be performed using MHC tetramer-based screening techniques.

### BRIEF DESCRIPTION OF THE DRAWINGS

FIG. 1A and FIG. 1B are graphs showing the effect of pTOP-OVA_CD8 prophylactic intramuscular immunization on the anti-tumor activity. FIG. 1A shows tumor growth follow-up after challenge. The tumor size was measured three times a week with an electronic digital caliper. Tumor volume was calculated as the length×width×height (in mm³). FIG. 1B shows survival rates monitoring after challenge. The asterisks indicate significant differences compared with naive mice (**P<0.01) (n=6) (Comparison of survival curves, Mantel-Cox test).

FIGS. 2A and 2B are graphs showing the effect of pTOP-OVA_CD8 therapeutic intratumoral immunization on the anti-tumor activity. FIG. 2A shows tumor growth follow-up after challenge. The tumor size was measured three times a week with an electronic digital caliper. Tumor volume was calculated as the length×width×height (in mm³). FIG. 2B shows survival rates monitoring after challenge. The asterisks indicate significant differences compared with naive mice (***P<0.001) (n=6) (Comparison of survival curves, Mantel-Cox test).

FIGS. 3A and 3B are graphs showing the effect of restriction sites addition around the inserted epitope sequence, for prophylactic intramuscular immunization. FIG. 3A shows tumor growth follow-up after challenge. The tumor size was measured three times a week with an electronic digital caliper. Tumor volume was calculated as the length×width×height (in mm³). FIG. 3B shows survival rates monitoring after challenge. The asterisks indicate significant differences compared with naive mice (**P<0.01) (n=6) (Comparison of survival curves, Mantel-Cox test).

FIGS. 4A and 4B are graphs showing the effect of pTOP1-OVA_CD8 and pTOP1-OVA_CD4 prophylactic intramuscular immunization on the anti-tumor activity. FIG. 4A shows tumor growth follow-up after challenge. The tumor size was measured three times a week with an electronic digital caliper. Tumor volume was calculated as the length×width×height (in mm³). FIG. 4B shows survival rates monitoring after challenge. The asterisks indicate significant differences compared with naive mice (***P<0.001) (n=6) (Comparison of survival curves, Mantel-Cox test).

FIG. 5A-D are graphs showing the effect of pTOP1-OVA_CD8 and pTOP1-OVA_CD4 therapeutic intramuscular immunization on the anti-tumor activity. FIG. 5A and FIG. 5C show tumor growth follow-up after challenge. FIG. 5B and FIG. 5D show survival rates monitoring after challenge. Survival curves were compared with a Mantel-Cox test. The asterisks indicate significant differences compared with naive mice (***P<0.001) (n=10 and n=6 respectively).

FIG. 6 is a graph showing the effect of co-delivery of pTOP1-OVA_CD4 with pTOP1-OVA_CD8 on the cytotoxic T cell response. Percentages of OVA target cell killing were compared and the asterisks indicate significant differences (***P<0.001) (n=5) (Student's T-test).

FIG. 7 is a graph showing an OTII proliferation assay and effect of immunization with MHC class II restricted epitope inserted in pTOP1. The percentages of cell division were compared by Student's T-test (***p<0.001) (n=5).

FIG. 8 is a set of graphs showing OTI proliferation assay and the effect of immunization with MHC class I restricted epitope inserted in pTOP1. The graph shows the percentages of cell division. The asterisks indicate significant differences (***P<0.001) (n=5) (Student's T-test).

FIGS. 9A and 9B are graphs showing the effect of pTOP1 intramuscular therapeutic immunization in combination with immune checkpoint blockade (ICB) therapy. FIG. 9A shows tumor growth follow-up after challenge. Tumor volume was calculated as the length×width×height (in mm³). FIG. 9B shows survival rates monitoring after challenge. The asterisks indicate significant differences between curves (*P<0.05; ***P<0.001) (n=6) (Comparison of survival curves, Mantel-Cox test).

FIGS. 10A and 10B are graphs showing the effect of pTOP1-OVA_CD4(18)_OVA_CD8(191) and pTOP1_gp100_CD4(18)_TRP2_CD8(191) therapeutic intramuscular immunization on the anti-tumor activity. FIG. 10A shows tumor growth follow-up after challenge. The tumor size was measured three times a week with an electronic digital caliper. Tumor volume was calculated as the length×width×height (in mm3). FIG. 10B shows survival rates monitoring after challenge. The asterisks indicate significant differences compared with naive mice (**P<0.01; ***P<0.001) (n=6) (Comparison of survival curves, Mantel-Cox test).

FIGS. 11A and 11B are graphs showing the effect of pTOP1-PADRE(18)_P1A_CD8(191) prophylactic intramuscular immunization on the anti-tumor activity. FIG. 11A shows tumor growth follow-up after challenge. The tumor size was measured three times a week with an electronic digital caliper. Tumor volume was calculated as the length×width×height (in mm3). FIG. 11B shows survival rates monitoring after challenge. The asterisks indicate significant differences compared with naive mice (**P<0.01) (n=6) (Comparison of survival curves, Mantel-Cox test).

FIG. 12 is a graph showing the effect of pTOP1-PADRE(18)_P1A_CD8(191) therapeutic intramuscular immunization on the anti-tumor activity. It indicates survival rate monitoring after challenge. The asterisk indicates significant differences compared with naive mice (*P<0.05) (n=6) (Comparison of survival curves, Mantel-Cox test).

FIGS. 13A and 13B are graphs showing the effect of pTOP1-PADRE(18)_AH1A5_CD8(191) prophylactic intramuscular immunization on the anti-tumor activity. FIG. 13A shows tumor growth follow-up after challenge. The tumor size was measured three times a week with an electronic digital caliper. Tumor volume was calculated as the length×width×height (in mm3). FIG. 13B shows survival rates monitoring after challenge. The asterisks indicate significant differences compared with naive mice (***P<0.001) (n=6) (Comparison of survival curves, Mantel-Cox test).

FIGS. 14A and 14B are graphs showing the effect of pTOP1-PADRE(18)_TRP2_CD8(191) prophylactic intramuscular immunization on the anti-tumor activity. FIG. 14A shows tumor growth follow-up after challenge. The tumor size was measured three times a week with an electronic digital caliper. Tumor volume was calculated as the length×width×height (in mm3). FIG. 14B shows survival rates monitoring after challenge. The asterisks indicate significant differences compared with naive mice (***P<0.001) (n=6) (Comparison of survival curves, Mantel-Cox test).

FIG. 15 is graph showing the effect of pTOP1-gp100_CD4(18)_OVA_CD8(191) and pTOP1_gp100_LP (18)_OVA_CD8(191) therapeutic intramuscular immunization on the anti-tumor activity. It indicates survival rates monitoring after challenge. The asterisks indicate significant differences compared with naive mice (*P<0.05; **P<0.01) (n=6) (Comparison of survival curves, Mantel-Cox test).

### EXAMPLES

The present invention is further illustrated by the following examples.

Materials and Methods

Material

Plasmids

Codon-optimized gene sequences of VSV-G (pTOP), VSV-G-OVA_CD8 (pTOP-OVA_CD8) and VSV-G-RS (with restriction sites, pTOP1) were designed using GeneOptimizer and obtained by standard gene synthesis from GeneArt® (Thermo Fisher Scientific, Waltham, Mass., US). These sequences were subcloned in the pVAX2 vector using cohesive-ends cloning. The pVAX2 vector consists of a pVAX1 plasmid (Invitrogen, Carlsbad, Calif.) in which the promoter was replaced by the pCMVβ plasmid promoter (Clontech, Palo Alto, Calif.). The plasmids were prepared using the EndoFree Plasmid Giga Kit (Qiagen, Venlo, Netherlands) according to the manufacturer's protocol. Plasmid dilutions were performed in Dulbecco's Phosphate Buffered Saline (1×) (PBS) (Life Technologies, Carlsbad, Calif., US). The quality of the purified plasmid was assessed by the ratio of optical densities (260 nm/280 nm) and by 0.5% agarose gel electrophoresis. DNA concentration was determined by optical density at 260 nm. The plasmids were stored at −20° C.

VSV-G Sequences Cloned in pVAX2
Vesicular stomatitis Indiana virus glycoprotein G (VSV-G) (SEQ ID NO: 1, encoded by SEQ ID NO: 10).
Plasmid nomenclature: pVAX2-VSVG (pTOP).

MKCLLYLAFLFIGVNCKFTIVFPHNQKGNWKNVPSNYHYCPSSSDLNWHN
DLIGTAIQVKMPKSHKAIQADGWMCHASKWVTTCDFRWYGPKYITQSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKGLCDSNLISM
DITFFSEDGELSSLGKEGTGFRSNYFAYETGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTTERELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK.

VSV-G (SEQ ID NO: 1) containing SIINFEKL sequence (OVA_CD8, SEQ ID NO: 11) at position 191 (SEQ ID NO: 8).
Plasmid nomenclature: pVAX2-VSVG-OVA_CD8 (pTOP-OVA_CD8).

MKCLLYLAFLFIGVNCKFTIVFPHNQKGNWKNVPSNYHYCPSSSDLNWHN
DLIGTAIQVKMPKSHKAIQADGWMCHASKWVTTCDFRWYGPKYITQSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKSIINFEKLGL
CDSNLISMDITFFSEDGELSSLGKEGTGFRSNYFAYETGGKACKMQYCKH
WGVRLPSGVWFEMADKDLFAAARFPECPEGSSISAPSQTSVDVSLIQDVE
RILDYSLCQETWSKIRAGLPISPVDLSYLAPKNPGTGPAFTIINGTLKYF
ETRYIRVDIAAPILSRMVGMISGTTTERELWDDWAPYEDVEIGPNGVLRT
SSGYKFPLYMIGHGMLDSDLHLSSKAQVFEHPHIQDAASQLPDDESLFFG
DTGLSKNPIELVEGWFSSWKSSIASFFFIIGLIIGLFLVLRVGIHLCIKL
KHTKKRQIYTDIEMNRLGK.

(in bold underlined is the OVA_CD8 sequence, SEQ ID NO: 11),
VSV-G (SEQ ID NO: 1) containing restriction sites (RS) at position 191 (SEQ ID NO: 9).
Plasmid nomenclature: pVAX2-VSVG-RS (pTOP1).

MKCLLYLAFLFIGVNCKFTIVFPHNQKGNWKNVPSNYHYCPSSSDLNWHN
DLIGTAIQVKMPKSHKAIQADGWMCHASKWVTTCDFRWYGPKYITQSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKTSEFGLCDSN
LISMDITFFSEDGELSSLGKEGTGFRSNYFAYETGGKACKMQYCKHWGVR
LPSGVWFEMADKDLFAAARFPECPEGSSISAPSQTSVDVSLIQDVERILD
YSLCQETWSKIRAGLPISPVDLSYLAPKNPGTGPAFTIINGTLKYFETRY
IRVDIAAPILSRMVGMISGTTTERELWDDWAPYEDVEIGPNGVLRTSSGY
KFPLYMIGHGMLDSDLHLSSKAQVFEHPHIQDAASQLPDDESLFFGDTGL
SKNPIELVEGWFSSWKSSIASFFFIIGLIIGLFLVLRVGIHLCIKLKHTK
KRQIYTDIEMNRLGK.

(In bold underlined are the SpeI/EcoRI restriction sites).

Peptide Insertion in pTOP1

To insert epitopes in position 191 of VSV-G (SEQ ID NO: 1), into the pTOP1 vector, cohesive-ends cloning was used. pVAX2-VSVG-RS was opened using SpeI and EcoRI and two complementary and overlapping phosphorylated oligonucleotides were incorporated. Multiple plasmids were obtained by varying the sequence of the oligonucleotides which were ordered from Eurogentec (Seraing, Belgium) or IDT-DNA (Leuven, Belgium). For peptide insertion in position 18 of pTOP1, Gibson Assembly Cloning Kit (New England BioLabs Inc.) with gBlocks gene fragments was used according to the manufacturer instructions. A HindIII restriction site was added for allowing easy peptide modification at the position 18. Plasmids were then purified, characterized and stored as explained here above.

TABLE 3
Peptides inserted in pTOP-1 by cohesive-
ends cloning at position 191 of SEQ ID NO: 1.
Peptide sequence, name and function are described.

| SEQ | | | |
|---|---|---|---|
| ID | | | |
| NO: | Peptide | Name | Function |
| 11 | SIINFEKL | OVA_CD8 | CD8 T cell |
| | | | epitope against |
| | | | ovalbumin |
| 12 | ISQAVHAAHAEINEAGR | OVA_CD4 | CD4 T cell |
| | | | epitope against |
| | | | ovalbumin |
| 13 | LPYLGWLVF | P1A_CD8 | CD8 T cell |
| | | | epitope against |
| | | | P1A |
| 14 | ELAGIGILTV | MELANA_CD8 | CD8 T cell |
| | | | epitope against |
| | | | MART-1 |
| 15 | IMDQVPFSV | GP100_CD8 | CD8 T cell |
| | | | epitope against |
| | | | gp100 |
| 16 | YMDGTMSQV | TYR_CD8 | CD8 T cell |
| | | | epitope against |
| | | | tyrosinase |
| 133 | SPSYAYHQF | AH1A5_CD8 | CD8 T cell |
| | | | epitope against |
| | | | gp70 |
| 134 | SVYDFFVWL | TRP2_CD8 | CD8 T cell |
| | | | epitope against |
| | | | TRP2 |

TABLE 4
Peptides inserted in pTOP1 by gBlocks
cloning at position 18 of SEQ ID NO: 1.
Peptide sequence, name and function are described.

| SEQ | | | |
|---|---|---|---|
| ID | | | |
| NO: | Peptide | Name | Function |
| 12 | ISQAVHAAH | OVA_CD4 | CD4 T cell |
| | AEINEAGR | | epitope against |
| | | | ovalbumin |
| 17 | AKFVAAW | PADRE | Universal antigenic |
| | TLKAAA | | CD4 T cell |
| | | | epitope against |
| | | | pan-HLA DR |
| 18 | VQGEESNDK | VIL1 | Universal antigenic |
| | | | CD4 T cell |
| | | | epitope from IL1β |
| 19 | QYIKANSK | TT | Universal antigenic |
| | FIGITEL | | CD4 T cell |
| | | | epitope from Tetanus |
| | | | toxoid |
| 20 | WNRQLYPE | GP100_CD4 | CD4 T cell |
| | WTEAQRLD | | epitope against |
| | | | gp100 |
| 21 | DPNAPKRPP | HP91 | Universal antigenic |
| | SAFFLFCSE | | CD4 T cell epitope |
| | | | against HMGB1- |
| | | | derived |
| | | | immunostimulatory |
| | | | peptide hp91 |
| 22 | KVPRNQDWL | GP100_LP | Long peptide |
| | GVSRQLRTK | | containing a CD8 |
| | AWNRQLYPE | | (underlined) and |
| | WTEAQRLD | | potential CD4 |
| | | | (italic) T cell |
| | | | epitopes against |
| | | | gp100 |
| 23 | NLLHRYSLE | P1A_LP | Long peptide |
| | EILPYLGWL | | containing a CD8 |
| | VFAVVTTSF | | (underlined) and |
| | LALQMFIDA | | potential CD4 |
| | LYEE | | T cell epitopes |
| | | | against P1A |

List of Constructs

TABLE 5
List of chimeric VSV-G used in the present invention. Given are
their amino acid sequence ID and nucleic acid sequence ID.

| | Nucleic | | |
|---|---|---|---|
| Protein | Acid | | |
| SEQ ID | SEQ ID | Name | Function |
| 38 | 24 | Modified VSV-G in | CD8 T cell epitope against ovalbumin in |
| | | pTOP1-OVA_CD4(18)- | position 191 and CD4 T cell epitope against |
| | | OVA_CD8(191) | ovalbumin in position 18 of VSV-G (SEQ ID |
| | | | NO: 1) in pTOP1 |
| 39 | 25 | Modified VSV-G in | CD8 T cell epitope against ovalbumin in |
| | | pTOP1-OVA_CD8(191) | position 191 of VSV-G (SEQ ID NO: 1) in |
| | | | pTOP1 |
| 40 | 26 | Modified VSV-G in | CD4 T cell epitope against ovalbumin in |
| | | pTOP1-OVA_CD4(191) | position 191 of VSV-G (SEQ ID NO: 1) in |
| | | | pTOP1 |
| 41 | 27 | Modified VSV-G in | CD8 T cell epitope against MART-1 in position |
| | | pTOP1- | 191 of VSV-G (SEQ ID NO: 1) in pTOP1 |
| | | MELANA_CD8(191) | |
| 42 | 28 | Modified VSV-G in | CD8 T cell epitope against gp100 in position |
| | | pTOP1- | 191 of VSV-G (SEQ ID NO: 1) in pTOP1 |
| | | GP100_CD8(191) | |
| 43 | 29 | Modified VSV-G in | CD8 T cell epitope against P1A in position 191 |
| | | pTOP1-P1A_CD8(191) | of VSV-G (SEQ ID NO: 1) in pTOP1 |
| 44 | 30 | Modified VSV-G in | CD8 T cell epitope against tyrosinase in |
| | | pTOP1-TYR_CD8(191) | position 191 of VSV-G (SEQ ID NO: 1) in |
| | | | pTOP1 |
| 45 | 31 | Modified VSV-G in | Universal antigenic CD4 T cell epitope against |
| | | pTOP1-PADRE(18)- | pan-HLA DR in position 18 and CD8 T cell |
| | | OVA_CD8(191) | epitope against ovalbumin in position 191 of |
| | | | VSV-G (SEQ ID NO: 1) in pTOP1 |
| 46 | 32 | Modified VSV-G in | Universal antigenic CD4 T cell epitope from |
| | | pTOP1-VIL1(18)- | IL1β in position 18 and CD8 T cell epitope |
| | | OVA_CD8(191) | against ovalbumin in position 191 of VSV-G |
| | | | (SEQ ID NO: 1) in pTOP1 |
| 47 | 33 | Modified VSV-G in | Universal antigenic CD4 T cell epitope from |
| | | pTOP1-TT(18)- | Tetanus toxoid in position 18 and CD8 T cell |
| | | OVA_CD8(191) | epitope against ovalbumin in position 191 of |
| | | | VSV-G (SEQ ID NO: 1) in pTOP1 |
| 48 | 34 | Modified VSV-G in | CD4 T cell epitope against gp100 in position 18 |
| | | pTOP1-GP100_CD4(18)- | and CD8 T cell epitope against ovalbumin in |
| | | OVA_CD8(191) | position 191 of VSV-G (SEQ ID NO: 1) in |
| | | | pTOP1 |
| 49 | 35 | Modified VSV-G in | HMGB1-derived immunostimulatory peptide |
| | | pTOP1-HP91(18)- | hp91 in position 18 and CD8 T cell epitope |
| | | OVA_CD8(191) | against ovalbumin in position 191 of VSV-G |
| | | | (SEQ ID NO: 1) in pTOP1 |
| 50 | 36 | Modified VSV-G in | Long peptide containing a CD8 and potential |
| | | pTOP1-P1A_LP(18)- | CD4 T cell epitopes against P1A in position 18 |
| | | OVA_CD8(191) | and CD8 T cell epitope against ovalbumin in |
| | | | position 191 of VSV-G (SEQ ID NO: 1) in |
| | | | pTOP1 |
| 51 | 37 | Modified VSV-G in | Long peptide containing a CD8 and potential |
| | | pTOP1-GP100_LP(18)- | CD4 T cell epitopes against gp100 in position |
| | | OVA_CD8(191) | 18 and CD8 T cell epitope against ovalbumin in |
| | | | position 191 of VSV-G (SEQ ID NO: 1) in |
| | | | pTOP1 |
| 135 | 139 | Modified VSV-G in | CD4 T cell epitope against gp100 in position 18 |
| | | pTOP1-GP100_CD4(18)- | and CD8 T cell epitope against TRP2 in |
| | | TRP2_CD8(191) | position 191 of VSV-G (SEQ ID NO: 1) in |
| | | | pTOP1 |
| 136 | 140 | Modified VSV-G in | Universal antigenic CD4 T cell epitope against |
| | | pTOP1-PADRE(18)- | pan-HLA DR in position 18 and CD8 T cell |
| | | P1A_CD8(191) | epitope against P1A in position 191 of VSV-G |
| | | | (SEQ ID NO: 1) in pTOP1 |
| 137 | 141 | Modified VSV-G in | Universal antigenic CD4 T cell epitope against |
| | | pTOP1-PADRE(18)- | pan-HLA DR in position 18 and CD8 T cell |
| | | AH1A5_CD8(191) | epitope against gp70 in position 191 of VSV-G |
| | | | (SEQ ID NO: 1) in pTOP1 |
| 138 | 142 | Modified VSV-G in | Universal antigenic CD4 T cell epitope against |
| | | pTOP1-PADRE(18)- | pan-HLA DR in position 18 and CD8 T cell |
| | | TRP2_CD8(191) | epitope against TRP2 in position 191 of VSV-G |
| | | | (SEQ ID NO: 1) in pTOP1 |

Cell Culture

B16F10-OVA, a melanoma cell line from C57BL/6 mice that stably expresses ovalbumin, was cultured in MEM medium supplemented with GlutaMAX with 10% FBS, 100 μg/mL streptomycin and 100 U/mL penicillin (Life Technologies, Carlsbad, Calif., US).

B16F10, a melanoma cell line from C57BL/6 mice, was cultured in MEM medium supplemented with GlutaMAX with 10% FBS, 100 μg/mL streptomycin and 100 U/mL penicillin (Life Technologies, Carlsbad, Calif., US).

CT26, a colon carcinoma cell line from BALB/C mice, was cultured in DMEM with 10% FBS, 100 μg/mL streptomycin and 100 U/mL penicillin, and supplemented with L-glutamate and pyruvate (Life Technologies, Carlsbad, Calif., US).

P815, a mastocytoma cell line from DBA/2 mice, was cultured in DMEM with 10% FBS, 100 μg/mL streptomycin and 100 U/mL penicillin (Life Technologies, Carlsbad, Calif., US).

Animals

Six to eight-week-old C57BL/6, BALB/C and DBA/2 female mice were obtained from Janvier Labs (Le Genest Saint Isle, FR) and housed in a minimal disease facility with ad libitum access to food and water.

For tumor implantation and electroporation, the mice were anaesthetized by intraperitoneal (ip) injection of 150 μL of a solution of 10 mg/mL ketamine and 1 mg/mL xylazine. The ethical committee for Animal Care and Use of the Medical Sector of the Université Catholique de Louvain approved our experimental protocols (UCL/MD/2011/007 and UCL/MD/2016/001).

Methods

Immunization

After removing the hair using a rodent shaver (AgnTho's, Lidingö, Sweden), 1 μg or 50 μg of plasmid were injected, diluted in 30 μL of PBS, into the left tibial cranial muscle. Immediately after injection, the leg was placed between 4-mm-spaced plate electrodes (BTX Caliper Electrodes), and 8 square-wave electric pulses (80 V, 20 ms, 2 Hz) were delivered by a Gemini System generator (BTX; both from VWR International, Leuven, Belgium). A conductive gel was used to ensure electrical contact with the skin (Aquasonic 100; Parker Laboratories, Inc., Fairfield, N.J., USA).

For prophylactic vaccination experiments, two boosts (i.e., second and third administrations of the vaccine) were similarly applied two and four weeks after the priming.

For therapeutic vaccination experiments, the treatment started two days after the injection of the tumor cells and the two boosts were delivered every week.

Alternatively, plasmids were injected and electroporated into the tumors when they reached a size in-between 30 and 50 mm³. This treatment was then repeated after two days.

For the study of the OT-I and OT-II proliferation, plasmids were injected into ears and 2-mm-spaced electrodes were applied to deliver 10 square-wave electric pulses (100 V, 20 ms, 1 Hz).

Tumor Implantation

1×10⁵ B16F10-OVA or B16F10 cells, diluted in 100 μL PBS, were injected subcutaneously into the right flank of each C57BL/6.

1×10⁶ CT26 cells, diluted in 100 μL PBS, were injected subcutaneously into the right flank of each BALB/C.

1×10⁶ P815 cells, diluted in 100 μL PBS, were injected subcutaneously into the right flank of each DBA/2.

Tumor cells were implanted two days before the first plasmid administration or two weeks after the last administration for therapeutic and prophylactic DNA immunization studies, respectively. The tumor size was measured three times a week with an electronic digital caliper. Tumor volume was calculated as the length×width×height (in mm³). The mice were sacrificed when the volume of the tumor reached 1500 mm³ or when they were in poor condition and expected to die shortly.

Administration of Immune Checkpoint Blockade (ICB) Antibodies

For administration of ICB, mice received 100 μg of InVivoMAb anti-mouse CTLA-4 (CD152) clone 9D9 and 100 μg of InVivoMAb anti-mouse PD-1 (CD279) clone 29F.1A12, both from BioXcell (CT, US) by intraperitoneal injection in 200 μL of PBS at day 3, 6 and 9 following implantation of the B16F10-OVA cells.

OT-I and OT-II Proliferation

T cells were isolated from spleen and lymph nodes of transgenic OT-I and OT-II mice using CD8+ and CD4+ T cell isolation kit II mouse (Miltenyi Biotec, The Netherlands). Subsequently the T cells were labeled with CFSE (carboxyfluorescein diacetate succinimidyl ester; Molecular probes) by incubating 50×10⁶ cells/mL with 5 μM CFSE for 7 minutes at 37° C. The reaction was blocked by adding ice-cold PBS (Lonza, Belgium)+10% serum. 2×10⁶ OT-I or OT-II cells were injected into the tail vein of C57BL/6 mice. They were treated 2 days later by plasmid injection and electroporation. Mice were sacrificed 4 days later to collect the draining lymph nodes for single cell suspension preparation. Flow cytometric measurement was performed after staining with aqua live dead (Invitrogen, Belgium), CD19 APC-Cy7, CD8 PerCP (all BD Biosciences), dextramer SIINFEKL H-2kb PE (Immudex, Denmark).

In Vivo Killing Assay

Splenocytes from naive mice were pulsed with SIINFEKL peptide or with an irrelevant peptide (40 μg in 40 mL PBS) for one hour at 37° C. Subsequently, these pulsed splenocytes were washed and respectively stained with high (5 μM, hi) or low (0.5 μM, low) CFSE concentration. The two populations of splenocytes were mixed in a 1:1 ratio, and 10⁷ splenocytes were intravenously injected into immunized mice two weeks after the last booster immunization. Two days after transfer, the spleens of the host mice were isolated and analyzed by flow cytometry after staining with α-F4/80 (BD Biosciences, San Diego, Calif., USA) to exclude auto-fluorescent macrophages. The percentage antigen-specific killing was determined using the following formula:

$$\% antigen specific killing = 100 - ( 100 \times \frac{{[ \frac{\% {CFSE}^{hi} cells}{\% {CFSE}^{low} cells} ]}^{immunized mice}}{{[ \frac{\% {CFSE}^{hi} cells}{\% {CFSE}^{low} cells} ]}^{non \text{-} immunized mice}} )$$

### Example 1: The Effect of pTOP-OVA_CD8(191) Prophylactic Intramuscular Immunization on the Anti-Tumor Activity

B16 melanoma is a spontaneous melanoma derived from C57BL/6 mice. The most commonly used variant is B16F10, which is highly aggressive and will metastasize from a primary subcutaneous site to the lungs, as well as colonize lungs upon intravenous (iv) injection.

C57BL/6 mice were immunized in a regimen of one prime and two boosts at a 2-week interval with the pTOP-OVA_CD8(191) plasmid (1 μg). Two weeks after the last vaccination, they were challenged with B16F10-OVA cells. This B16F10-OVA cell line is a stable transfectant derived from B16F10 melanoma that stably expresses chicken ovalbumin.

Tumor growth and mouse survival were assessed for three months.

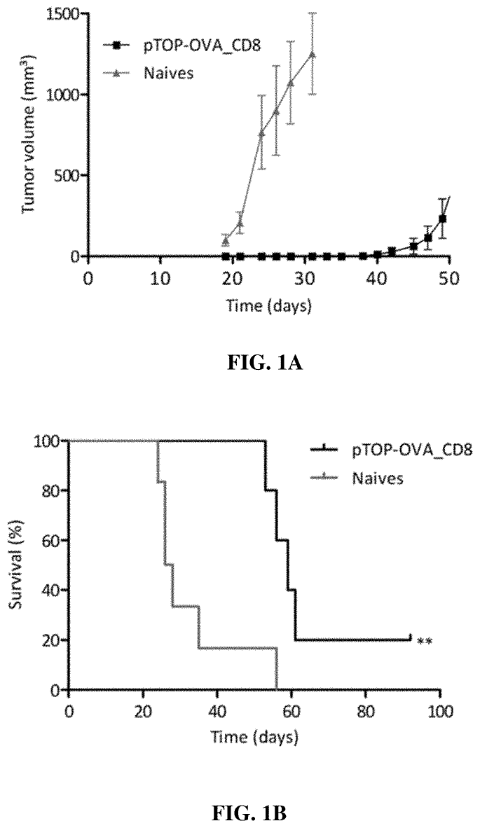

Inoculation of B16F10-OVA cells induced tumors that grow rapidly and killed naïve mice. However, prophylactic immunization by intramuscular electroporation of a plasmid encoding VSV-G containing a tumor model CD8 T cell epitope delayed tumor growth and improved mice survival (FIGS. 1A and 1B).

### Example 2: The Effect of pTOP-OVA_CD8(191) Therapeutic Intratumoral Immunization on the Anti-Tumor Activity

C57BL/6 mice were challenged with B16F10-OVA cells. When tumor reached between 30 and 50 mm³, mice were immunized twice with a two-day interval with the pTOP-OVA_CD8(191) plasmid, the pTOP control plasmid (expressing VSV-G of SEQ ID NO: 1 without inserted peptide) or the empty pVAX2 (pEmpty) plasmid (50 μg each).

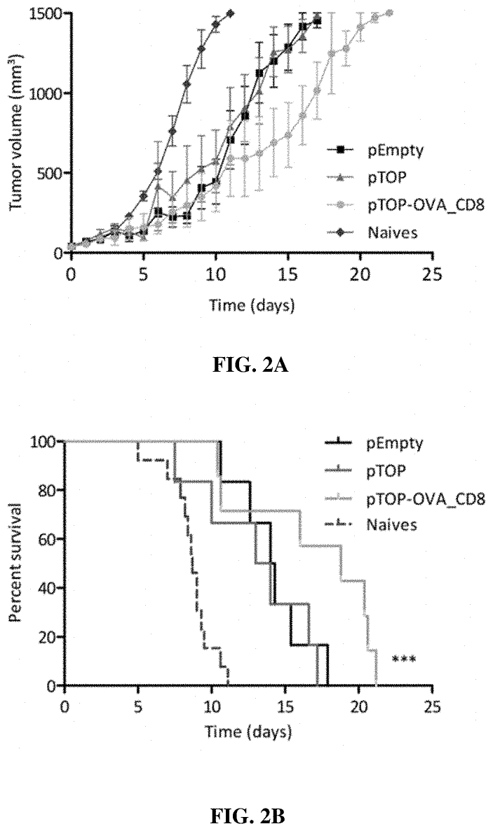

Therapeutic immunization by intratumoral electroporation of a plasmid encoding VSV-G containing a tumor model CD8 T cell epitope delays tumor growth (FIGS. 2A and 2B).

### Example 3: The Effect of Restriction Sites Addition Around the Inserted Epitope Sequence on Vaccine Efficacy

C57BL/6 mice were immunized in a regimen of one prime and two boosts at a 2-week interval with the pTOP-OVA_CD8(191) plasmid or the pTOP1-OVA_CD8(191) plasmid (1 μg each). Two weeks after the last vaccination, they were challenged with B16F10-OVA cells. Tumor growth and mouse survival were assessed.

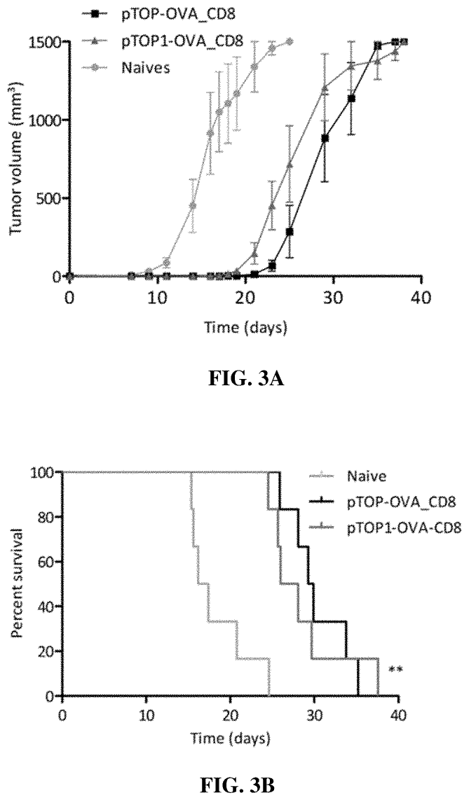

The addition of SpeI and EcoRI restriction sites introduce amino acids TS and EF around the inserted epitope. This result showed that adding these amino acids around the T cell epitope does not alter vaccine efficacy (FIGS. 3A and 3B).

### Example 4: The Effect of pTOP1-OVA_CD8(191) and pTOP1-OVA_CD4(191) Prophylactic Intramuscular Immunization on the Anti-Tumor Activity

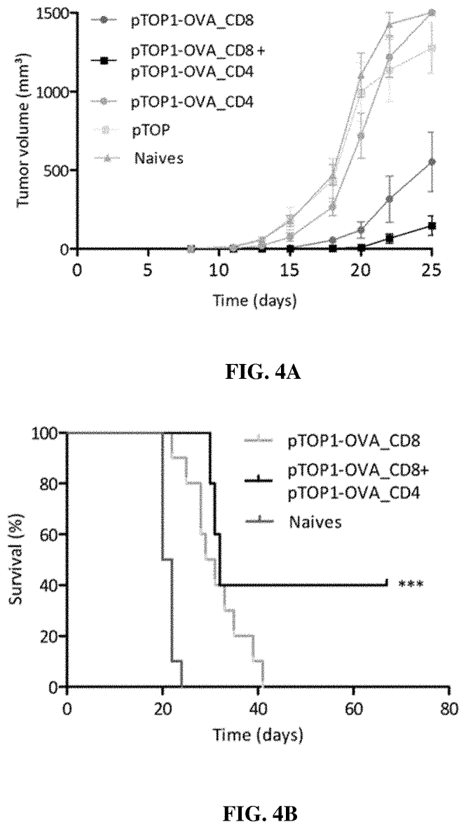

Insertion of a CD8 T cell epitope in VSV-G is necessary to observe anti-tumor efficacy. There is no anti-tumor effect following pTOP and pTOP1-OVA_CD4(191) delivery. Prophylactic immunization by intramuscular electroporation of two pTOP1 plasmids containing respectively OVA_CD8 and OVA_CD4 T cell epitopes improve protection against tumor challenge as compared to pTOP1-OVA_CD8(191) alone. The tumor growth delay and mice survival are improved when the helper epitope is co-delivered with the MHC class I restricted epitope (FIGS. 4A and 4B).

### Example 5: The Effect of pTOP1-OVA_CD8(191) and pTOP1-OVA_CD4(191) Therapeutic Intramuscular Immunization on the Anti-Tumor Activity

C57BL/6 mice were challenged with B16F10-OVA cells. Two days later, they were immunized in a regimen of one prime and two boosts at a 1-week interval with 1 μg of the pTOP1-OVA_CD8(191) alone or combined with 1 μg of the pTOP1-OVA_CD4(191) plasmid. Tumor growth and mouse survival were assessed.

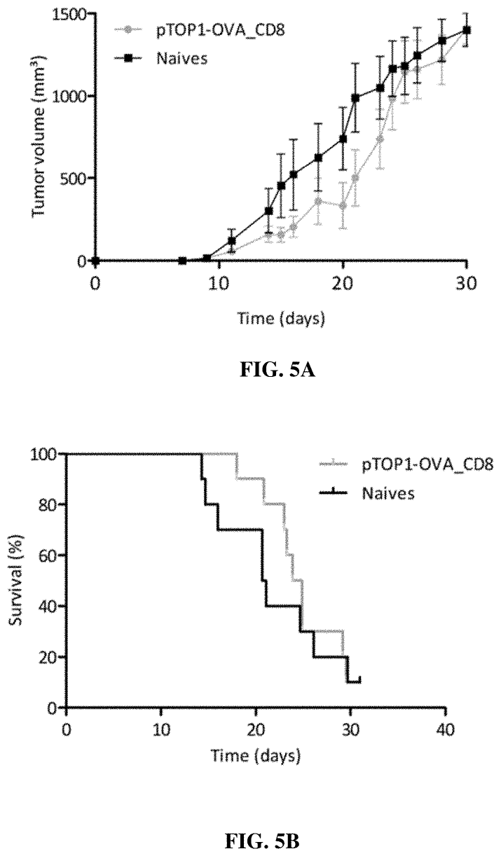

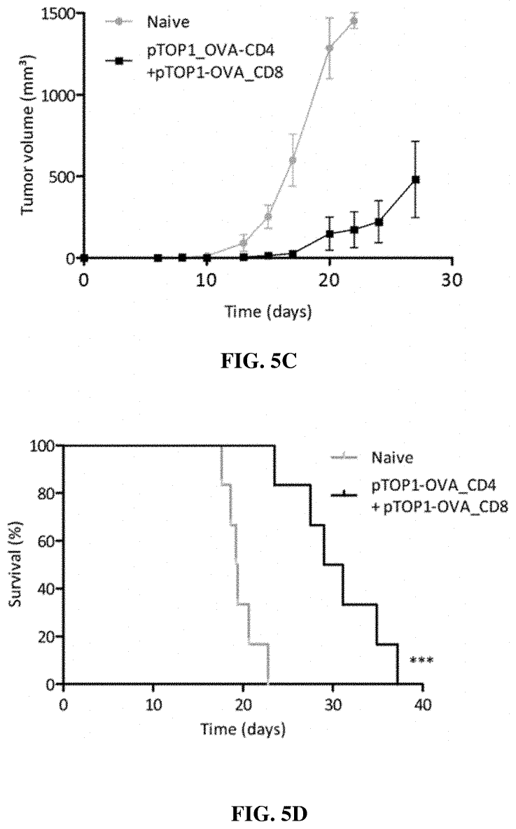

Therapeutic immunization by intramuscular electroporation of two pTOP1 plasmids containing respectively CD8 and CD4 T cell epitopes improves protection against tumor challenge. Two separate experiments have been performed. First, it was shown that therapeutic immunization with pTOP1-OVA_CD8(191) tends to improve protection against challenge (but the effect is not significant). Second, the combination of pTOP1-OVA_CD4(191) and pTOP1-OVA_CD8(191) drastically improved mice survival and delayed tumor growth (FIG. 5A-D).

### Example 6: The Effect of Co-Delivery of pTOP1-OVA_CD4(191) with pTOP-OVA_CD8(191) on the Cytotoxic T Cell Response

C57BL/6 mice were immunized in a regimen of one prime and two boosts at a 2-week interval with 1 μg of the pTOP1-OVA_CD8(191) plasmid alone or combined with 1 μg of the pTOP1-OVA_CD4(191) plasmid. The percentage of antigen specific killing was analyzed by in vivo cytotoxic assay. Immunized mice were adoptively transferred with two populations of labelled splenocytes: MHC-I OVA peptide-pulsed-target cells and a MHC-I irrelevant-peptide-pulsed cells. Two days after transfer, the specific killing of target cells was obtained by comparing the relative decrease of the two populations.

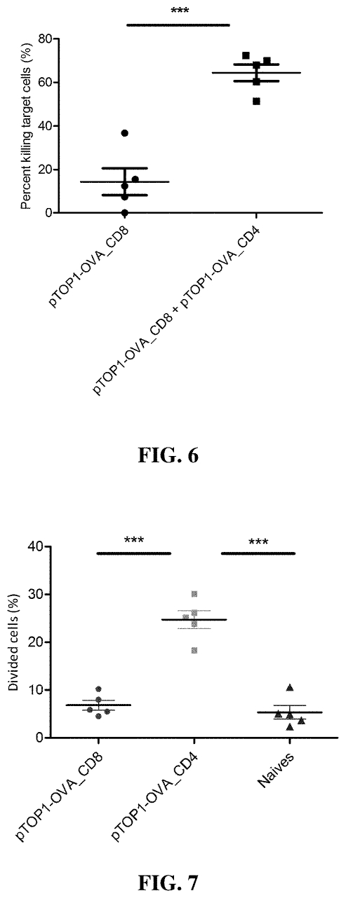

An in vivo killing assay demonstrated that co-delivery of pTOP1-OVA_CD8(191) and pTOP1-OVA_CD4(191) improves the cytotoxic T cell response to the vaccine antigen as compared to delivery of pTOP1-OVA_CD8(191) alone (FIG. 6).

### Example 7: OT-II Proliferation Assay

The effect of immunization with MHC class II-restricted epitope inserted in pTOP1 on the CD4+ T cell response has been demonstrated using OT-II cells. T cells were isolated from spleen and lymph nodes of transgenic OT-II mice, labeled with CFSE and adoptively transferred to C57BL/6 mice. Mice were immunized two days later with 1 μg of pTOP1-OVA_CD4(191) or 1 μg of pTOP1-OVA_CD8(191). Mice were sacrificed four days later and labelled T cell proliferation was assessed.

The insertion of MHC class II-restricted epitopes in VSV-G-induced CD4+ T cell response, whereas MHC class I-restricted epitopes are unable to induce helper response (FIG. 7).

### Example 8: OT-I Proliferation Assay

The effect of immunization with MHC class I-restricted epitope inserted in pTOP1 on the CD8+ T cell response has been demonstrated using OT-I cells. T cells were isolated from spleen and lymph nodes of transgenic OT-I mice, labeled with CFSE and adoptively transferred to receptor C57BL/6 mice. Mice were immunized two days later by electroporation of pTOP1-OVA_CD4(191) (1 μg) or pTOP1-OVA_CD8(191) (1 μg). Mice were sacrificed four days later and labelled T cell proliferation was assessed.

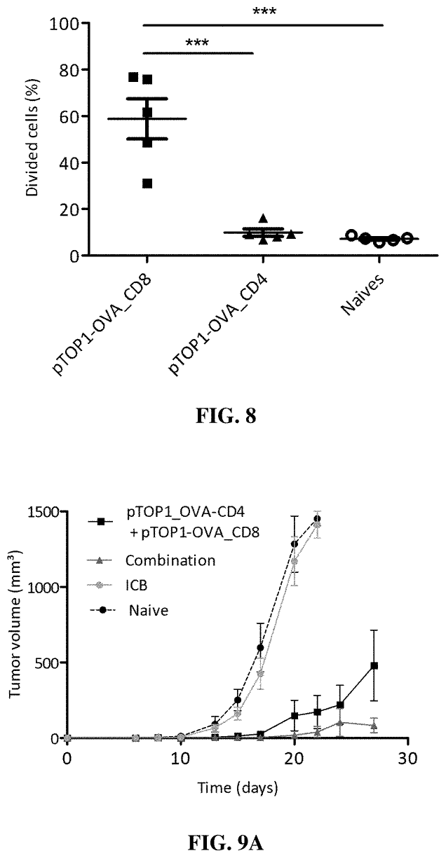

The insertion of MHC class I-restricted epitopes in VSV-G induced CD8+ T cell response, whereas MHC class II-restricted epitopes are unable to induce CD8+ T cell response (FIG. 8).

### Example 9: The Effect of pTOP1 Immunization in Combination with Immune Checkpoint Blockade (ICB) Therapy

C57BL/6 mice were challenged with B16F10-OVA cells. Two days later, they were immunized in a regimen of one prime and two boosts at a 1-week interval. On day 3, 6 and 9 following challenge, the ICB treatments were given. Mice received either
(1) both pTOP1-OVA_CD8(191) (1 μg) and pTOP1-OVA_CD4(191) (1 μg) plasmids;
(2) a cocktail of anti-PD-1 and anti-CTLA-4 antibodies [ICB group]; or
(3) a combination of the two plasmids (1 μg each) and the antibodies cocktail [combination group].

Tumor growth and mice survival were assessed following challenge.

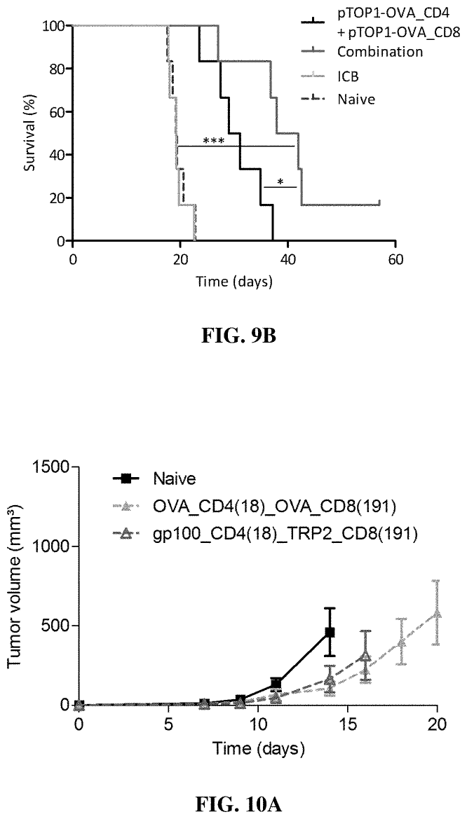

Efficacy of pTOP1 is further enhanced by combination with immune checkpoint blockade therapy. These results demonstrated that the combinatory treatment has a synergic effect compared to treatments alone. Indeed, survival, tumor growth and tumor volume observed after the combinatory treatment are better than the sum of effects obtained after separate treatments (FIGS. 9A and 9B).

### Example 10: The Effect of pTOP1-OVA_CD4(18)_OVA_CD8(191) and pTOP1-Gp100_CD4(18)_TRP2_CD8(191) Therapeutic Intramuscular Immunization on the Anti-Tumor Activity

C57BL/6 mice were challenged with B16F10-OVA cells. Two days later, they were immunized in a regimen of one prime and two boosts at a 1-week interval with 1 μg of the pTOP1-OVA_CD4(18)_OVA_CD8(191) plasmid or 1 μg of the pTOP1-gp100_CD4(18)_TRP2_CD8(191) plasmid. Tumor growth and mouse survival were assessed.

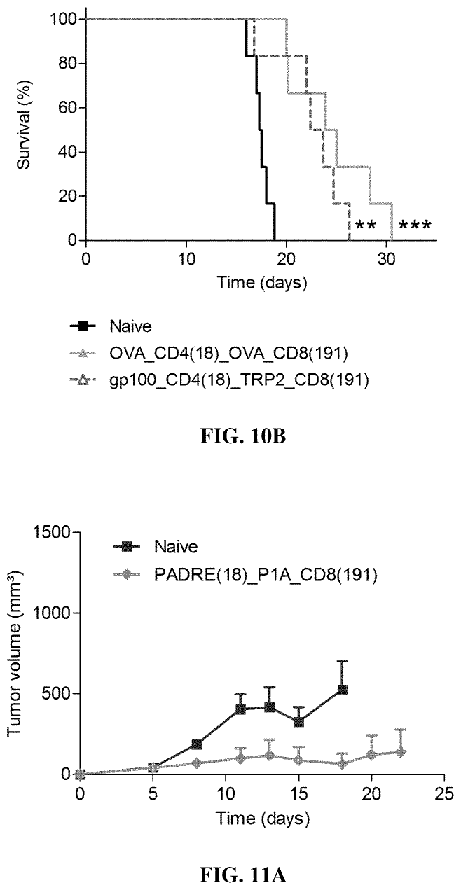

Therapeutic immunization by intramuscular electroporation of pTOP1-OVA_CD4(18)_OVA_CD8(191) plasmid or pTOP1-gp100_CD4(18)_TRP2_CD8(191) was able to significantly delay tumor growth. There was no statistical difference between the two vaccines (FIGS. 10A and 10B).

### Example 11: The Effect of pTOP1-PADRE(18)_P1A_CD8(191) Prophylactic Intramuscular Immunization on the Anti-Tumor Activity

DBA/2 mice were immunized in a regimen of one prime and two boosts at a 2-week interval with the pTOP1-PADRE(18)_P1A_CD8(191) plasmid (1 μg). Two weeks after the last vaccination, they were challenged with P815 cells. Tumor growth and mouse survival were assessed for two months.

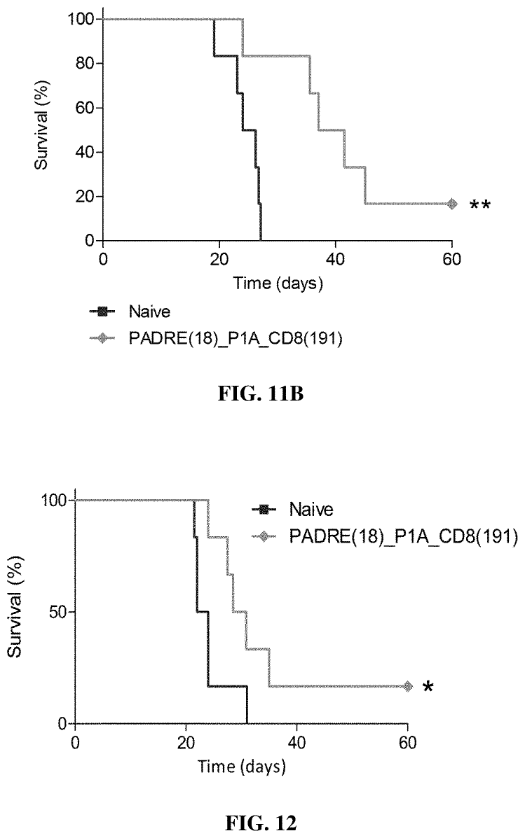

Inoculation of P815 cells induced tumors that grow rapidly and killed naïve mice. However, prophylactic immunization by intramuscular electroporation of a plasmid encoding VSV-G containing a tumor model CD8 T cell epitope and a universal antigenic CD4 T cell epitope delayed tumor growth and improved mice survival (FIGS. 11A and 11B).

### Example 12: The Effect of pTOP1-PADRE(18)_P1A_CD8(191) Therapeutic Intramuscular Immunization on the Anti-Tumor Activity

DBA/2 mice were challenged with P815 cells. Two days later, they were immunized in a regimen of one prime and two boosts one and two weeks later with the pTOP1-PADRE(18)_P1A_CD8(191) plasmid (1 μg). Mice survival was assessed for two months.

Therapeutic immunization by intramuscular electroporation of pTOP1-PADRE(18)_P1A_CD8(191) plasmid was able to significantly delay tumor growth. (FIG. 12).

### Example 13: The Effect of pTOP1-PADRE(18)_AH1A5_CD8(191) Prophylactic Intramuscular Immunization on the Anti-Tumor Activity

BALB/C mice were immunized in a regimen of one prime and two boosts at a 2-week interval with the pTOP1-PADRE(18)_AH1A5_CD8(191) plasmid (1 μg). Two weeks after the last vaccination, they were challenged with CT26 cells. Tumor growth and mouse survival were assessed for two months.

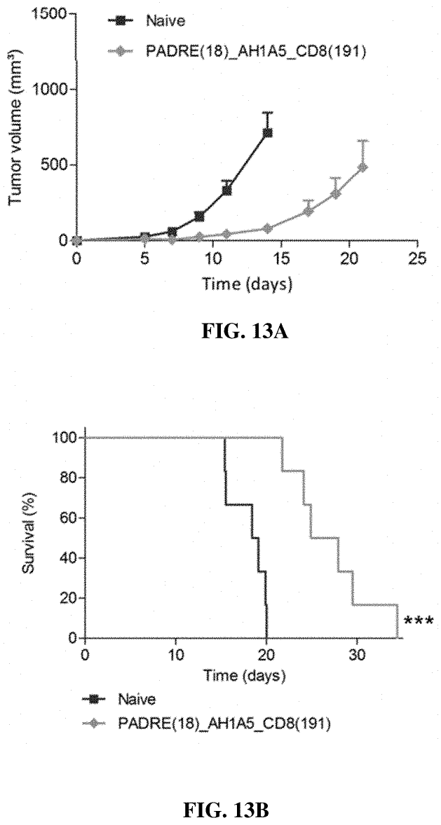

Inoculation of CT26 cells induced tumors that grow rapidly and killed naïve mice. However, prophylactic immunization by intramuscular electroporation of a plasmid encoding VSV-G containing a tumor model CD8 T cell epitope and a universal antigenic CD4 T cell epitope delayed tumor growth (FIGS. 13A and 13B).

### Example 14: The Effect of pTOP1-PADRE(18)_TRP2_CD8(191) Prophylactic Intramuscular Immunization on the Anti-Tumor Activity

BALB/C mice were immunized in a regimen of one prime and two boosts at a 2-week interval with the pTOP1-PADRE(18)_TRP2_CD8(191) plasmid (1 μg). Two weeks after the last vaccination, they were challenged with B16F10 cells. Tumor growth and mouse survival were assessed for two months.

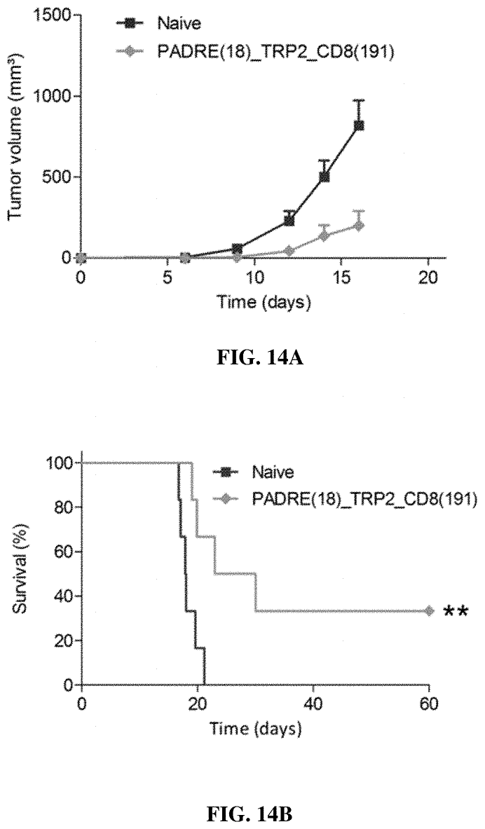

Inoculation of B16F10 cells induced tumors that grow rapidly and killed naïve mice. However, prophylactic immunization by intramuscular electroporation of a plasmid encoding VSV-G containing a tumor model CD8 T cell epitope and a universal antigenic CD4 T cell epitope delayed tumor growth and improved mice survival (FIGS. 14A and 14B).

### Example 15: The Effect of pTOP1-Gp100_CD4(18)_OVA_CD8(191) and pTOP1-Gp100_LP(18)_OVA_CD8(191) Therapeutic Intramuscular Immunization on the Anti-Tumor Activity

C57BL/6 mice were challenged with B16F10-OVA cells. Two days later, they were immunized in a regimen of one prime and two boosts at a 1-week interval with 1 μg of the pTOP1-gp100_CD4(18)_OVA_CD8(191) plasmid or 1 μg of the pTOP1-gp100_LP(18)_OVA_CD8(191) plasmid. Tumor growth and mouse survival were assessed.

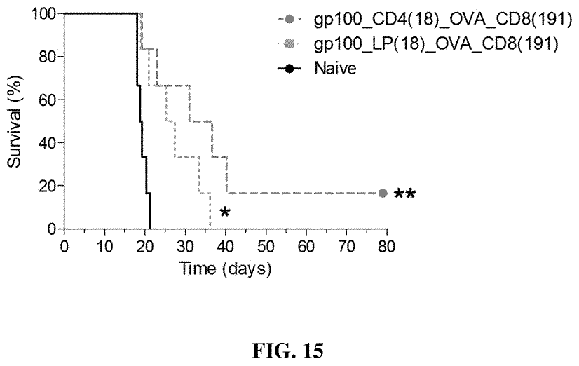

Therapeutic immunization by intramuscular electroporation of pTOP1-gp100_CD4(18)_OVA_CD8(191) plasmid or pTOP1-gp100_LP(18)_OVA_CD8(191) was able to significantly delay tumor growth. There was no statistical difference between the two vaccines (FIG. 15).

1-15. (canceled)

16. An isolated nucleic acid sequence coding for a modified vesicular stomatitis virus glycoprotein (VSV-G) comprising at least one tumor antigen or fragment thereof.

17. The isolated nucleic acid sequence coding for a modified VSV-G according to claim 16, wherein said at least one tumor antigen or fragment thereof comprises at least one epitope.

18. The isolated nucleic acid sequence coding for a modified VSV-G according to claim 16, wherein said at least one tumor antigen or fragment thereof is a neoantigen.

19. The isolated nucleic acid sequence coding for a modified VSV-G according to claim 16, wherein said at least one antigen or fragment thereof is inserted into VSV-G at an amino acid position selected from the group consisting of positions 18, 51, 55, 191, 196, 217, 368 and C-terminal, and combinations thereof, wherein position numbering is with respect to vesicular stomatitis Indiana virus (VSIV) glycoprotein amino acid sequence.

20. The isolated nucleic acid sequence coding for a modified VSV-G according to claim 16, wherein said at least one antigen or fragment thereof is inserted into VSV-G at amino acid position 18 or 191 and combinations thereof, wherein position numbering is with respect to vesicular stomatitis Indiana virus (VSIV) glycoprotein amino acid sequence.

21. The isolated nucleic acid sequence coding for a modified VSV-G according to claim 16, wherein said VSV-G is from vesicular stomatitis Indiana virus (VSIV).

22. The isolated nucleic acid sequence coding for a modified VSV-G according to claim 16, wherein said VSV-G has a sequence identity of at least 70% with SEQ ID NO: 1.

23. The isolated nucleic acid sequence coding for a modified VSV-G according to claim 16, wherein said VSV-G comprises or consists of SEQ ID NO: 1.

24. A modified vesicular stomatitis virus glycoprotein (VSV-G) comprising at least one tumor antigen or fragment thereof.

25. A vaccine comprising a modified vesicular stomatitis virus glycoprotein (VSV-G) comprising at least one antigen or fragment thereof, a nucleic acid sequence coding therefor, a vector containing a nucleic acid sequence coding therefor, or a dendritic cell population transfected by a nucleic acid sequence coding therefor.

26. The vaccine according to claim 25 comprising at least one adjuvant.

27. The vaccine according to claim 25, wherein said vaccine is a polynucleotide vaccine.

28. The vaccine according to claim 25, wherein said vaccine is a protein vaccine.

29. A method for preventing and/or treating a disease or condition in a subject in need thereof comprising administering to said subject a modified vesicular stomatitis virus glycoprotein (VSV-G) comprising at least one antigen or fragment thereof, a nucleic acid sequence coding therefor, a vector containing a nucleic acid sequence coding therefor, a dendritic cell population transfected by a nucleic acid sequence coding therefor, or a vaccine comprising said modified VSV-G, nucleic acid sequence, vector or dendritic cell population and optionally at least one adjuvant.

30. The method according to claim 29, wherein said vaccine is a polynucleotide vaccine.

31. The method according to claim 29, wherein said vaccine is a protein vaccine.

32. The method according to claim 29, wherein said disease is a cancer.

33. The method according to claim 29, wherein said disease is an infectious disease.

34. The method according to claim 29, wherein said modified VSV-G, nucleic acid sequence, vector, dendritic cell population or vaccine is administered to the subject by intramuscular injection, intradermal injection, intratumoral injection, peritumoral injection, gene gun, electroporation or sonoporation.

35. The method according to claim 29, wherein said modified VSV-G, nucleic acid sequence, vector, dendritic cell population or vaccine is administered before, concomitantly or after one or more checkpoint blockade antibodies.


SEQUENCE LISTING (148 sequences)

SEQ ID NO: 1 - protein, 511 aa, Vesicular stomatitis Indiana virus
  VSIV-G
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 517 aa, Vesicular stomatitis New Jersey virus
  VSNJV-G
MLSYLIFALVVSPILGKIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYKDGVSFNPGFPPQSCGYGTVTDAEAHIVTVTPHSVKVDEYTGEWIDPHFIGGRCKGQICETVHNSTKWFTSSDGESVCSQLFTLVGGTFFSDSEEITSMGLPETGIRSNYFPYVSTEGICKMPFCRKPGYKLKNDLWFQITDPDLDKTVRDLPHIKDCDLSSSIVTPGEHATDISLISDVERILDYALCQNTWSKIEAGEPITPVDLSYLGPKNPGAGPVFTIINGSLHYFMSKYLRVELESPVIPRMEGKVAGTRIVRQLWDQWFPFGEVEIGPNGVLKTKQGYKFPLHIIGTGEVDNDIKMERIVKHWEHPHIEAAQTFLKKDDTEEVLYYGDTGVSKNPVELVEGWFSGWRSSIMGVLAVIIGFVILIFLIRLIGVLSSLFRQKRRPIYKSDVEMAHFR

SEQ ID NO: 3 - protein, 530 aa, Chandipura virus
  CHPV-G
MLSQVTLQAFVVMCLIYRAYSSLSIAFPESTKLDWKPVTKNTRYCPMGGEWFLEPGLQEESFLSSTPIGATPSKSDGFLCHAAKWVTTCDFRWYGPKYITHSIHNIKPTRSDCDSALASYKSGTLINPGFPPDSCGYASVTDSEFLVIMITPHHVGVDDYRGHWVDPLFVGGECDQSYCDTIHNSSVWIPADTTKKNICGQSFTPLTVTVAYDKTKDISAGALVFKSKYHSHMEGARTCRLTYCGRTGIKFPNGEWVSLDIKTKIQDKQLLPLFKECPAGTEVRSTLQSDGAQVLTSEIQRLLDYSLCQNTWEKVDRKEPLSPLDLSYLASKSPGKGLAYTVINGTLSFAHTRYVRMWIDGPVLKELKGKRESASGIASDIWTQWFKYGDMEIGPNGLLKTKSGYKFPWHLIGMGIVDNELHELSEANPLDHPQLPHAQSIADDSEEIFFGDTGVSKNPVELVTGWFTSWKESLAAGVVLILTVVLIYGVLRCFPVLCMPCRKTKWKKEVERSDSFEMRIFKPNNMRARV

SEQ ID NO: 4 - protein, 512 aa, Cocal virus
  COCV-G
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 5 - protein, 529 aa, Piry virus
  PIRYV-G
MDLFPILVVVLMTDTVLGKFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 6 - protein, 523 aa, Isfahan virus
  ISFV-G
MTSVLFMVGVLLGAFGSTHCSIQIVFPSETKLVWKPVLKGTRYCPQSAELNLEPDLKTMAFDSKVPIGITPSNSDGYLCHAAKWVTTCDFRWYGPKYITHSVHSLRPTVSDCKAAVEAYNAGTLMYPGFPPESCGYASITDSEFYVMLVTPHPVGVDDYRGHWVDPLFPTSECNSNFCETVHNATMWIPKDLKTHDVCSQDFQTIRVSVMYPQTKPTKGADLTLKSKFHAHMKGDRVCKMKFCNKNGLRLGNGEWIEVGDEVMLDNSKLLSLFPDCLVGSVVKSTLLSEGVQTALWETDRLLDYSLCQNTWEKIDRKEPLSAVDLSYLAPRSPGKGMAYIVANGSLMSAPARYIRVWIDSPILKEIKGKKESASGIDTVLWEQWLPFNGMELGPNGLIKTKSGYKFPLYLLGMGIVDQDLQELSSVNPVDHPHVPIAQAFVSEGEEVFFGDTGVSKNPIELISGWFSDWKETAAALGFAAISVILIIGLMRLLPLLCRRRKQKKVIYKDVELNSFDPRQAFHR

SEQ ID NO: 7 - protein, 509 aa, Spring viraemia of carp virus
  SVCV-G
MSIISYIAFLLLIDSTLGIPIFVPSGQNISWQPVIQPFDYQCPIHGNLPNTMGLSATKLTIKSPSVFSTDKVSGWICHAAEWKTTCDYRWYGPQYITHSIHPISPTIDECKRIISRIASGTDEDLGFPPQSCGWASVTTVSNTNYKVVPHSVHLEPYGGHWIDHEFNGGECREKVCEMKGNHSIWITDETVQHECEKHIEEVEGIMYGNAPRGDALYINNFIIDKHHRVYRFGGSCRMKFCDKDGIKFTRGDWVEKTAETLTKIYANTPECADGTLVSGHRPGLDLIDTVFNLENVVEYTLCEGTKRKINEQEKLTSVDLSYLAPRIGGFGSVFRVRNGTLERGSTTYIRIEVEGPIVDSLTGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGTTLVALILIFLLIRCCVACTYLMKKSKRPATESHEMRSLV

SEQ ID NO: 8 - protein, 519 aa, Artificial Sequence
  Modified VSV-G-OVA_CD8 in pTOP
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKSIINFEKLGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 9 - protein, 515 aa, Artificial Sequence
  Modified VSV-G-RS from pTOP1
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 10 - DNA, 1536 bp, Vesicular stomatitis Indiana virus
  VSIV-G
atgaagtgcctgctgtacctggccttcctgttcatcggcgtgaactgcaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcatcaatggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 11 - protein, 8 aa, Artificial Sequence
  OVA_CD8
SIINFEKL

SEQ ID NO: 12 - protein, 17 aa, Artificial Sequence
  OVA_CD4
ISQAVHAAHAEINEAGR

SEQ ID NO: 13 - protein, 9 aa, Artificial Sequence
  P1A_CD8
LPYLGWLVF

SEQ ID NO: 14 - protein, 10 aa, Artificial Sequence
  MELANA_CD8
ELAGIGILTV

SEQ ID NO: 15 - protein, 9 aa, Artificial Sequence
  GP100_CD8
IMDQVPFSV

SEQ ID NO: 16 - protein, 9 aa, Artificial Sequence
  TYR_CD8
YMDGTMSQV

SEQ ID NO: 17 - protein, 13 aa, Artificial Sequence
  PADRE
AKFVAAWTLKAAA

SEQ ID NO: 18 - protein, 9 aa, Artificial Sequence
  VIL1
VQGEESNDK

SEQ ID NO: 19 - protein, 15 aa, Artificial Sequence
  TT
QYIKANSKFIGITEL

SEQ ID NO: 20 - protein, 16 aa, Artificial Sequence
  GP100_CD4
WNRQLYPEWTEAQRLD

SEQ ID NO: 21 - protein, 18 aa, Artificial Sequence
  HP91
DPNAPKRPPSAFFLFCSE

SEQ ID NO: 22 - protein, 35 aa, Artificial Sequence
  GP100_LP
KVPRNQDWLGVSRQLRTKAWNRQLYPEWTEAQRLD

SEQ ID NO: 23 - protein, 40 aa, Artificial Sequence
  P1A_LP
NLLHRYSLEEILPYLGWLVFAVVTTSFLALQMFIDALYEE

SEQ ID NO: 24 - DNA, 1632 bp, Artificial Sequence
  Modified VSV-G OVA_CD4(18)-OVA_CD8(191) in pTOP1
atgaagtgcctgctgtacctggccttcctgttcatcggcgtgaactgcaagatcagccaggccgtgcacgctgcccacgccgagatcaatgaggccggcagaaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 25 - DNA, 1572 bp, Artificial Sequence
  Modified VSV-G OVA_CD8(191) in pTOP1
atgaagtgcctgctgtacctggccttcctgttcatcggcgtgaactgcaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 26 - DNA, 1599 bp, Artificial Sequence
  Modified VSV-G OVA_CD4(191) in pTOP1
atgaagtgcctgctgtacctggccttcctgttcatcggcgtgaactgcaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtatcagccaggccgtgcacgctgcccacgccgagatcaatgaggccggcagagaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 27 - DNA, 1578 bp, Artificial Sequence
  Modified VSV-G MELANA_CD8(191) in pTOP1
atgaagtgcctgctgtacctggccttcctgttcatcggcgtgaactgcaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtgaactggctggcatcggcatcctgacagtggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 28 - DNA, 1575 bp, Artificial Sequence
  Modified VSV-G GP100_CD8(191) in pTOP1
atgaagtgcctgctgtacctggccttcctgttcatcggcgtgaactgcaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtatcatggatcaggtgcccttcagcgtggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 29 - DNA, 1575 bp, Artificial Sequence
  Modified VSV-G P1A_CD8(191) in pTOP1
atgaagtgcctgctgtacctggccttcctgttcatcggcgtgaactgcaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtctgccctacctgggctggctggtgttcgaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 30 - DNA, 1575 bp, Artificial Sequence
  Modified VSV-G TYR_CD8(191) in pTOP1
atgaagtgcctgctgtacctggccttcctgttcatcggcgtgaactgcaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttacatggacggcaccatgagccaggtggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 31 - DNA, 1620 bp, Artificial Sequence
  Modified VSV-G PADRE(18)-OVA_CD8(191) in pTOP1
atgaagtgcctgctgtacctggccttcctgttcatcggcgtgaactgcaaggccaagttcgtggccgcctggacactgaaggctgccgctaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 32 - DNA, 1608 bp, Artificial Sequence
  Modified VSV-G VIL1(18)-OVA_CD8(191) in pTOP1
atgaagtgcctgctgtacctggccttcctgttcatcggcgtgaactgcaaggtgcagggcgaggaaagcaacgacaagaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 33 - DNA, 1626 bp, Artificial Sequence
  Modified VSV-G TT(18)-OVA_CD8(191) in pTOP1
atgaagtgcctgctgtacctggccttcctgttcatcggcgtgaactgcaagcagtacatcaaggccaacagcaagttcatcggcatcaccgagctgaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 34 - DNA, 1629 bp, Artificial Sequence
  Modified VSV-G GP100_CD4(18)-OVA_CD8(191) in pTOP1
atgaagtgcctgctgtacctggccttcctgttcatcggcgtgaactgcaagtggaacagacagctgtaccccgagtggaccgaggcccagagactggataagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 35 - DNA, 1635 bp, Artificial Sequence
  Modified VSV-G HP91(18)-OVA_CD8(191) in pTOP1
atgaagtgcctgctgtacctggccttcctgttcatcggcgtgaactgcaaggaccccaacgcccctaagaggcctcccagcgcattcttcctgttctgcagcgagaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 36 - DNA, 1701 bp, Artificial Sequence
  Modified VSV-G P1A_LP(18)-OVA_CD8(191) in pTOP1
atgaagtgcctgctgtacctggccttcctgttcatcggcgtgaactgcaagaacctgctgcacagatacagcctggaagagatcctgccctacctgggctggctggtgttcgccgtcgtgacaacatccttcctggccctgcagatgttcatcgacgccctgtacgaggaaaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 37 - DNA, 1686 bp, Artificial Sequence
  VSV-G in pTOP1-GP100_LP(18)-OVA_CD8(191)
atgaagtgcctgctgtacctggccttcctgttcatcggcgtgaactgcaagaaggtgcccagaaaccaggactggctgggcgtgtccagacagctgagaacaaaggcctggaacaggcagctgtaccccgagtggacagaggcccagagactggacaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagttccatcatcaacttcgagaagctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 38 - protein, 543 aa, Artificial Sequence
  Modified VSV-G OVA_CD4(18)-OVA_CD8(191) in pTOP1
MKCLLYLAFLFIGVNCKISQAVHAAHAEINEAGRKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 39 - protein, 523 aa, Artificial Sequence
  Modified VSV-G OVA_CD8(191) in pTOP1
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 40 - protein, 532 aa, Artificial Sequence
  Modified VSV-G OVA_CD4(191) in pTOP1
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSISQAVHAAHAEINEAGREFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 41 - protein, 525 aa, Artificial Sequence
  Modified VSV-G MELANA_CD8(191) in pTOP1
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSELAGIGILTVEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 42 - protein, 524 aa, Artificial Sequence
  Modified VSV-G GP100_CD8(191) in pTOP1
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSIMDQVPFSVEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 43 - protein, 524 aa, Artificial Sequence
  Modified VSV-G P1A_CD8(191) in pTOP1
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSLPYLGWLVFEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 44 - protein, 524 aa, Artificial Sequence
  Modified VSV-G TYR_CD8(191) in pTOP1
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSYMDGTMSQVEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 45 - protein, 539 aa, Artificial Sequence
  Modified VSV-G PADRE(18)-OVA_CD8(191) in pTOP1
MKCLLYLAFLFIGVNCKAKFVAAWTLKAAAKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 46 - protein, 535 aa, Artificial Sequence
  Modified VSV-G VIL1(18)-OVA_CD8(191) in pTOP1
MKCLLYLAFLFIGVNCKVQGEESNDKKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 47 - protein, 541 aa, Artificial Sequence
  Modified VSV-G TT(18)-OVA_CD8(191) in pTOP1
MKCLLYLAFLFIGVNCKQYIKANSKFIGITELKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 48 - protein, 542 aa, Artificial Sequence
  Modified VSV-G GP100_CD4(18)-OVA_CD8(191) in pTOP1
MKCLLYLAFLFIGVNCKWNRQLYPEWTEAQRLDKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 49 - protein, 544 aa, Artificial Sequence
  Modified VSV-G HP91(18)-OVA_CD8(191) in pTOP1
MKCLLYLAFLFIGVNCKDPNAPKRPPSAFFLFCSEKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 50 - protein, 566 aa, Artificial Sequence
  Modified VSV-G P1A_LP(18)-OVA_CD8(191) in pTOP1
MKCLLYLAFLFIGVNCKNLLHRYSLEEILPYLGWLVFAVVTTSFLALQMFIDALYEEKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 51 - protein, 561 aa, Artificial Sequence
  Modified VSV-G GP100_LP(18)-OVA_CD8(191) in pTOP1
MKCLLYLAFLFIGVNCKKVPRNQDWLGVSRQLRTKAWNRQLYPEWTEAQRLDKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSIINFEKLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 52 - protein, 16 aa, Artificial Sequence
  Signal peptide from VSV-G
MKCLLYLAFLFIGVNC

SEQ ID NO: 53 - protein, 17 aa, Artificial Sequence
  Signal peptide from Gaussia luciferase
MGVKVLFALICIAVAEA

SEQ ID NO: 54 - protein, 511 aa, Vesicular stomatitis Alagoas virus
  VSAV-G
MTPAFILCMLLAGSSWAKFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 55 - protein, 523 aa, Carajas virus
  CJSV-G
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 56 - protein, 512 aa, Maraba virus
  MARAV-G
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 57 - protein, 22 aa, Artificial Sequence
  Signal peptide of Mus musculus Ig kappa light chain precursor (mutant A2)
MDMRAPAGIFGFLLVLFPGYRS

SEQ ID NO: 58 - protein, 18 aa, Artificial Sequence
  Signal peptide of Homo sapiens serum albumin preproprotein
MKWVTFISLLFLFSSAYS

SEQ ID NO: 59 - protein, 19 aa, Artificial Sequence
  Signal peptide of Homo sapiens immunoglobulin heavy chain
MDWTWRVFCLLAVTPGAHP

SEQ ID NO: 60 - protein, 19 aa, Artificial Sequence
  Signal peptide of Homo sapiens immunoglobulin light chain
MAWSPLFLTLITHCAGSWA

SEQ ID NO: 61 - protein, 19 aa, Artificial Sequence
  Signal peptide of Homo sapiens azurocidin preproprotein
MTRLTVLALLAGLLASSRA

SEQ ID NO: 62 - protein, 20 aa, Artificial Sequence
  Signal peptide of Homo sapiens Cystatin-S precursor
MARPLCTLLLLMATLAGALA

SEQ ID NO: 63 - protein, 15 aa, Artificial Sequence
  Signal peptide of Pseudopleuronectes americanus trypsinogen 2 precursor
MRSLVFVLLIGAAFA

SEQ ID NO: 64 - protein, 22 aa, Artificial Sequence
  Signal peptide of Mesobuthus martensii potassium channel blocker
MSRLFVFILIALFLSAIIDVMS

SEQ ID NO: 65 - protein, 21 aa, Artificial Sequence
  Signal peptide of Conus leopardus α-conotoxin lp1.3
MGMRMMFIMFMLVVLATTVVS

SEQ ID NO: 66 - protein, 18 aa, Artificial Sequence
  Signal peptide of Saccharomyces cerevisiae α-galactosidase (mutant m3)
MRAFLFLTACISLPGVFG

SEQ ID NO: 67 - protein, 18 aa, Artificial Sequence
  Signal peptide of Aspergillus niger cellulase
MKFQSTLLLAAAAGSALA

SEQ ID NO: 68 - protein, 24 aa, Artificial Sequence
  Signal peptide of Nepenthes gracilis aspartic proteinase nepenthesin-1
MASSLYSFLLALSIVYIFVAPTHS

SEQ ID NO: 69 - protein, 26 aa, Artificial Sequence
  Signal peptide of Nepenthes rafflesiana acid chitinase
MKTHYSSAILPILTLFVFLSINPSHG

SEQ ID NO: 70 - protein, 36 aa, Artificial Sequence
  Signal peptide of M28 virus K28 prepro-toxin
MESVSSLFNIFSTIMVNYKSLVLALLSVSNLKYARG

SEQ ID NO: 71 - protein, 21 aa, Artificial Sequence
  Signal peptide of Zygosaccharomyces bailii killer toxin zygocin precursor
MKAAQILTASIVSLLPIYTSA

SEQ ID NO: 72 - protein, 19 aa, Artificial Sequence
  Signal peptide of Vibrio cholerae O139 cholera toxin
MIKLKFGVFFTVLLSSAYA

SEQ ID NO: 73 - protein, 50 aa, Artificial Sequence
  Signal peptide of Saccharomyces cerevisiae- derived adhesion subunit of  α-agglutinin
MQLLRCFSIFSVIASVLAQELTTICEQIPSPTLESTPYSLSTTTILANGK

SEQ ID NO: 74 - protein, 51 aa, Artificial Sequence
  Signal peptide of Saccharomyces cerevisiae- derived exo-1,3-β glucanase
MLSLKTLLCTLLTVSSVLATPVPARDPSSIQFVHEENKKRYYDYDHGSLGE

SEQ ID NO: 75 - protein, 61 aa, Artificial Sequence
  Signal peptide of Saccharomyces cerevisiae- derived mating pheromone α-factor
MRFPSIFTAVLFAASSALAAPVNTTTEDETAQIPAEAVIGYLDLEGDFDVAVLPFSNSTNN

SEQ ID NO: 76 - protein, 67 aa, Artificial Sequence
  Signal peptide of Saccharomyces cerevisiae- derived chitin trans-glycosylase
MKVLDLLTVLSASSLLSTFAAAESTATADSTTAASSTASCNPLKTTGCTPDTALATSFSEDFSSSSK

SEQ ID NO: 77 - protein, 66 aa, Artificial Sequence
  Signal peptide of Saccharomyces cerevisiae- derived phospholipase B
MKLQSLLVSAAVLTSLTENVNAWSPNNSYVPANVTCDDDINLVREASGLSDNETEWLKKRDAYTKE

SEQ ID NO: 78 - protein, 100 aa, Artificial Sequence
  Signal peptide of Saccharomyces cerevisiae- derived cell wall protein related to glucanases
MKLSATTLTAASLIGYSTIVSALPYAADIDTGCTTTAHGSHQHKRAVAVTYVYETVTVDKNGQTVTPTSTEASSTVASTTTLISESSVTKSSSKVASSSE

SEQ ID NO: 79 - protein, 97 aa, Artificial Sequence
  Signal peptide of Saccharomyces cerevisiae- derived phospholipase B
MQLRNILQASSLISGLSLAADSSSTTGDGYAPSIIPCPSDDTSLVRNASGLSTAETDWLKKRDAYTKEALHSFLSRATSNFSDTSLLSTLFSSNSSN

SEQ ID NO: 80 - protein, 51 aa, Artificial Sequence
  Signal peptide of Saccharomyces cerevisiae- derived exo-1,3-β glucanase
MISPISFLSSLLCLTYLTSALPILPKREVVTRVHTASTTNVVTDFYSTTTE

SEQ ID NO: 81 - protein, 100 aa, Artificial Sequence
  Signal peptide of Saccharomyces cerevisiae- derived cell wall-associated protein involved in export of acetylated sterols
MLEFPISVLLGCLVAVKAQTTFPNFESDVLNEHNKFRALHVDTAPLTWSDTLATYAQNYADQYDCSGVLTHSDGPYGENLALGYTDTGAVDAWYGEISKY

SEQ ID NO: 82 - protein, 88 aa, Artificial Sequence
  Signal peptide of Saccharomyces cerevisiae- derived aspartic protease
MKLKTVRSAVLSSLFASQVLGKIIPAANKRDDDSNSKFVKLPFHKLYGDSLENVGSDKKPEVRLLKRADGYEEIIITNQQSFYSVDLE

SEQ ID NO: 83 - protein, 62 aa, Artificial Sequence
  Signal peptide of Saccharomyces cerevisiae- derived cell wall mannoprotein
MVKLTSIAAGVAAIAATASATTTLAQSDERVNLVELGVYVSDIRAHLAQYYSFQVAHPTETY

SEQ ID NO: 84 - protein, 65 aa, Artificial Sequence
  Signal peptide of Saccharomyces cerevisiae- derived cell wall mannoprotein
MVKLTSIVAGVAAIAAGVAAAPATTTLSPSDERVNLVELGVYVSDIRAHLAEYYMFQAAHPTETY

SEQ ID NO: 85 - protein, 38 aa, Artificial Sequence
  Signal peptide of Saccharomyces cerevisiae- derived mating pheromone α-factor
MQPITTASTQATQKDKSSEKKDNYIIKGLFWDPACVIA

SEQ ID NO: 86 - protein, 53 aa, Artificial Sequence
  Signal peptide of Saccharomyces cerevisiae- derived sporulation-specific exo-1,3-b-glucanase
MVSFRGLTTLTLLFTKLVNCNPVSTKNRDSIQFIYKEKDSIYSAINNQAINEK

SEQ ID NO: 87 - protein, 18 aa, Artificial Sequence
  Signal peptide of Homo sapiens chymotrypsinogen
MAFLWLLSCWALLGTTFG

SEQ ID NO: 88 - protein, 14 aa, Artificial Sequence
  Signal peptide of Homo sapiens interleukin-2
MQLLSCIALILALV

SEQ ID NO: 89 - protein, 15 aa, Artificial Sequence
  Signal peptide of Homo sapiens trypsinogen-2
MNLLLILTFVAAAVA

SEQ ID NO: 90 - protein, 17 aa, Artificial Sequence
  Signal peptide of Metridia longa luciferase
MDIKVVFTLVFSALVQA

SEQ ID NO: 91 - protein, 16 aa, Artificial Sequence
  Signal peptide of Oikopleura dioica Oikosin 1
MLLLSALLLGLAHGYS

SEQ ID NO: 92 - protein, 18 aa, Artificial Sequence
  Signal peptide of Oikopleura dioica Oikosin 2A
MKLLASVLTIAAADYACC

SEQ ID NO: 93 - protein, 21 aa, Artificial Sequence
  Signal peptide of Oikopleura dioica Oikosin 3
MKISAGLLGVALGQNEGSAEA

SEQ ID NO: 94 - protein, 16 aa, Artificial Sequence
  Signal peptide of Oikopleura dioica Oikosin 4A
MKLFAALSAFSASVEA

SEQ ID NO: 95 - protein, 14 aa, Artificial Sequence
  Signal peptide of Oikopleura dioica Oikosin 5A
MKLLCSVLLGTVFG

SEQ ID NO: 96 - protein, 14 aa, Artificial Sequence
  Signal peptide of Oikopleura dioica Oikosin 6A
MKISPLLVVTAVVG

SEQ ID NO: 97 - protein, 18 aa, Artificial Sequence
  Signal peptide of Oikopleura dioica Oikosin 7A
MKIAATFAALASATEWQG

SEQ ID NO: 98 - protein, 18 aa, Artificial Sequence
  Signal peptide of Vargula hilgendorfii luciferase
MKIIILSVILAYCVTDNC

SEQ ID NO: 99 - protein, 28 aa, Artificial Sequence
  Signal peptide of Methanococcus jannaschii Slmj1
MAMSLKKIGAIAVGGAMVATALASGVAA

SEQ ID NO: 100 - protein, 22 aa, Artificial Sequence
  Signal peptide of Hepatitis C virus serotype 1b E1 protein
MGCSFSIFLLALLSCLTTPASA

SEQ ID NO: 101 - protein, 20 aa, Artificial Sequence
  Signal peptide of Hepatitis C virus serotype 1b E2 protein
MVGNWAKVLIVMLLFAGVDG

SEQ ID NO: 102 - protein, 25 aa, Artificial Sequence
  Signal peptide of tissue plasminogen activator
MDAMKRGLCCVLLLCGAVFVDSVTG

SEQ ID NO: 103 - protein, 19 aa, Artificial Sequence
  Artificial signal peptide
MDAMKVLLLVFVSPSQVTG

SEQ ID NO: 104 - protein, 9 aa, Artificial Sequence
  CLPP_CD8
ILDKVLVHL

SEQ ID NO: 105 - protein, 9 aa, Artificial Sequence
  Cyclin-A1_CD8
FLDRFLSCM

SEQ ID NO: 106 - protein, 11 aa, Artificial Sequence
  Cyclin-A1_CD8
SLIAAAAFCLA

SEQ ID NO: 107 - protein, 10 aa, Artificial Sequence
  MAGE-A1_CD8
KEADPTGHSY

SEQ ID NO: 108 - protein, 9 aa, Artificial Sequence
  MAGE-A1_CD8
RVRFFFPSL

SEQ ID NO: 109 - protein, 10 aa, Artificial Sequence
  MAGE-C1_CD8
ILFGISLREV

SEQ ID NO: 110 - protein, 9 aa, Artificial Sequence
  MAGE-C1_CD8
KVVEFLAML

SEQ ID NO: 111 - protein, 9 aa, Artificial Sequence
  MAGE-C2_CD8
ASSTLYLVF

SEQ ID NO: 112 - protein, 15 aa, Artificial Sequence
  MAGE-C2_CD8
SSTLYLVFSPSSFST

SEQ ID NO: 113 - protein, 11 aa, Artificial Sequence
  SSX2_CD8
FGRLQGISPKI

SEQ ID NO: 114 - protein, 9 aa, Artificial Sequence
  XAGE1b/GAGED2a_CD8
RQKKIRIQL

SEQ ID NO: 115 - protein, 16 aa, Artificial Sequence
  XAGE1b/GAGED2a_CD8
HLGSRQKKIRIQLRSQ

SEQ ID NO: 116 - protein, 30 aa, Artificial Sequence
  Melan-A/MART-1_CD8
YTTAEEAAGIGILTVILGVLLLIGCWYCRR

SEQ ID NO: 117 - protein, 15 aa, Artificial Sequence
  TRP-1_CD8
SQWRVVCDSLEDYDT

SEQ ID NO: 118 - protein, 11 aa, Artificial Sequence
  Tyrosinase_CD8
IYMDGTADFSF

SEQ ID NO: 119 - protein, 9 aa, Artificial Sequence
  CD45_CD8
KFLDALISL

SEQ ID NO: 120 - protein, 9 aa, Artificial Sequence
  glypican-3_CD8
FVGEFFTDV

SEQ ID NO: 121 - protein, 9 aa, Artificial Sequence
  glypican-3_CD8
EYILSLEEL

SEQ ID NO: 122 - protein, 9 aa, Artificial Sequence
  IGF2B3_CD8
NLSSAEVVV

SEQ ID NO: 123 - protein, 9 aa, Artificial Sequence
  IGF2B3_CD8
RLLVPTQFV

SEQ ID NO: 124 - protein, 9 aa, Artificial Sequence
  Kallikrein 4_CD8
FLGYLILGV

SEQ ID NO: 125 - protein, 9 aa, Artificial Sequence
  KIF20A_CD8
LLSDDDVVV

SEQ ID NO: 126 - protein, 10 aa, Artificial Sequence
  KIF20A_CD8
AQPDTAPLPV

SEQ ID NO: 127 - protein, 9 aa, Artificial Sequence
  KIF20A_CD8
CIAEQYHTV

SEQ ID NO: 128 - protein, 10 aa, Artificial Sequence
  Lengsin_CD8
FLPEFGISSA

SEQ ID NO: 129 - protein, 13 aa, Artificial Sequence
  Meloa_CD8
RISSTLNDECWPA

SEQ ID NO: 130 - protein, 14 aa, Artificial Sequence
  Meloa_CD8
CPPWHPSERISSTL

SEQ ID NO: 131 - protein, 9 aa, Artificial Sequence
  MUC5AC_CD8
TCQPTCRSL

SEQ ID NO: 132 - protein, 15 aa, Artificial Sequence
  survivin_CD8
TLGEFLKLDRERAKN

SEQ ID NO: 133 - protein, 9 aa, Artificial Sequence
  AH1A5_CD8
SPSYAYHQF

SEQ ID NO: 134 - protein, 9 aa, Artificial Sequence
  TRP2_CD8
SVYDFFVWL

SEQ ID NO: 135 - protein, 543 aa, Artificial Sequence
  VSV-G in pTOP1-GP100_CD4(18)-TRP2_CD8(191)
MKCLLYLAFLFIGVNCKWNRQLYPEWTEAQRLDKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSVYDFFVWLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 136 - protein, 540 aa, Artificial Sequence
  VSV-G in pTOP1-PADRE(18)-P1A_CD8(191)
MKCLLYLAFLFIGVNCKAKFVAAWTLKAAAKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSLPYLGWLVFEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 137 - protein, 540 aa, Artificial Sequence
  VSV-G in pTOP1-PADRE(18)-AH1A5_CD8(191)
MKCLLYLAFLFIGVNCKAKFVAAWTLKAAAKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSPSYAYHQFEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 138 - protein, 540 aa, Artificial Sequence
  VSV-G in pTOP1-PADRE(18)-TRP2_CD8(191)
MKCLLYLAFLFIGVNCKAKFVAAWTLKAAAKLKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKTSSVYDFFVWLEFGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 139 - DNA, 1632 bp, Artificial Sequence
  VSV-G in pTOP1-GP100_CD4(18)-TRP2_CD8(191)
atgaagtgcctgctgtacctggccttcctgttcatcggcgtgaactgcaagtggaacagacagctgtaccccgagtggaccgaggcccagagactggataagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtagcgtgtacgacttcttcgtgtggctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 140 - DNA, 1623 bp, Artificial Sequence
  VSV-G in pTOP1-PADRE(18)-P1A_CD8(191)
atgaagtgcctgctgtacctggccttcctgttcatcggcgtgaactgcaaggccaagttcgtggccgcctggacactgaaggctgccgctaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtctgccctacctgggctggctggtgttcgaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 141 - DNA, 1623 bp, Artificial Sequence
  VSV-G in pTOP1-PADRE(18)-AH1A5_CD8(191)
atgaagtgcctgctgtacctggccttcctgttcatcggcgtgaactgcaaggccaagttcgtggccgcctggacactgaaggctgccgctaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtagccctagctacgcctaccaccagttcgaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 142 - DNA, 1623 bp, Artificial Sequence
  VSV-G in pTOP1-PADRE(18)-TRP2_CD8(191)
atgaagtgcctgctgtacctggccttcctgttcatcggcgtgaactgcaaggccaagttcgtggccgcctggacactgaaggctgccgctaagcttaagtttaccatcgtgttcccccacaaccagaagggcaactggaagaacgtgcccagcaactaccactactgccccagcagcagcgacctgaactggcacaacgacctgatcggcaccgccatccaagtgaagatgcccaagagccacaaggccatccaggccgatggctggatgtgccacgccagcaaatgggtcaccacctgtgacttcaggtggtacggccccaagtacatcacccagagcatcagatccttcacccccagcgtggaacagtgcaaagagagcatcgagcagaccaagcagggcacctggctgaaccccggattcccacctcagagctgtggctacgccaccgtgacagatgccgaggccgtgatcgtgcaagtgacccctcaccacgtgctggtggacgagtacacaggcgagtgggtggacagccagttcatcaacggcaagtgctccaactacatctgccccaccgtgcacaacagcaccacctggcacagcgactacaaagtgaagactagtagcgtgtacgacttcttcgtgtggctggaattcggcctgtgcgacagcaacctgatcagcatggacatcacattcttcagcgaggacggcgagctgagcagcctgggcaaagagggcacaggcttcagaagcaactacttcgcctacgagacaggcggcaaggcctgcaagatgcagtattgcaagcactggggcgtgcggctgcctagcggagtgtggttcgagatggccgacaaggacctgttcgccgctgccagattccccgagtgtcctgagggcagcagcatctctgcccctagccagacaagcgtggacgtgtccctgatccaggacgtggaaagaatcctggactacagcctgtgtcaggaaacctggtccaagatcagagccggcctgcccatcagccctgtggacctgtcttacctggcccccaagaaccctggaaccggccctgccttcaccatcattaacggcaccctgaagtactttgagacacggtacatccgggtggacattgccgcccctatcctgagcagaatggtgggaatgatcagcggcaccaccaccgagcgcgagctgtgggatgattgggccccttacgaggatgtggaaatcggccccaacggcgtgctgagaaccagcagcggctacaagttccccctgtacatgatcggccacggcatgctggactccgacctgcacctgtctagcaaggcccaggtgttcgagcacccccacatccaggatgccgccagccagctgcctgacgacgagtctctgttcttcggcgacaccggcctgagcaagaaccccatcgagctggtggaaggctggttcagcagctggaagtcctctatcgccagcttcttcttcatcatcgggctgattatcggcctgttcctggtgctgagagtgggcatccacctgtgcatcaagctgaagcacaccaagaagaggcagatctacaccgacatcgagatgaaccggctgggcaaatga

SEQ ID NO: 143 - protein, 25 aa, Artificial Sequence
  NY-ESO-1_CD4
LLEFYLAMPFATPMEAELARRSLAQ

SEQ ID NO: 144 - protein, 12 aa, Artificial Sequence
  listeriolysin O protein of Listeria monocytogenes
NEKYAQAYPNVS

SEQ ID NO: 145 - protein, 15 aa, Artificial Sequence
  Influenza Virus Nucleoprotein
QVYSLIRPNENPAHK

SEQ ID NO: 146 - protein, 11 aa, Artificial Sequence
  lymphocytic choriomeningitis virus (LCMV) glycoprotein (GP)
SGVENPGGYCL

SEQ ID NO: 147 - protein, 9 aa, Artificial Sequence
  lymphocytic choriomeningitis virus (LCMV) glycoprotein (GP)
KAVYNFATM

SEQ ID NO: 148 - protein, 9 aa, Artificial Sequence
  immunodominant adeno-associated virus 2 (AAV2)
VPQYGYLTL